(19) United States
(12) Patent Application Publication
(10) Pub. No.: US 2008/0274959 A1
(43) Pub. Date: Nov. 6, 2008

(54) Actinomadura Chromoprotein, Apoprotein and Gene Cluster

(75) Inventors: Bradley A. Haltli, Monroe, NY (US); Haiyin He, Mahwah, NJ (US); Ying Huang, Wayland, MA (US); Sridhar Krishna Rabindran, Malvern, PA (US); Jiang Wu, Lexington, MA (US); Min He, Congers, NY (US)

Correspondence Address: WYETH;PATENT LAW GROUP, 5 GIRALDA FARMS, MADISON, NJ, 07940, US

(73) Assignee: Wyeth, Madison, NJ (US)

(21) Appl. No.: 11/794,068

(22) PCT Filed: Dec. 16, 2005
(86) PCT No.: PCT/US2005/045818

§ 371 Date: Nov. 28, 2007

Related U.S. Application Data
(60) Provisional application No. 60/637,391, filed on Dec. 17, 2004.

(51) Int. Cl. A61K 38/16; C07H 21/04; C12N 15/63; C12N 1/21; C12N 1/15; C12P 21/00; C07H 17/08; C07K 14/37; G01N 33/566; C12N 1/14; G01N 33/53; A61P 35/00

(52) U.S. Cl. 514/12; 536/23.74; 435/320.1; 435/252.35; 435/254.11; 435/252.33; 435/69.1; 536/24.5; 436/501; 435/254.1; 435/7.2; 435/7.31; 536/7.1

(57) ABSTRACT

The present invention provides a chromoprotein produced by Actinomadura sp. 21G792, as well as amino acid and nucleic acid sequences of the apoprotein component of the chromoprotein and of components of the biosynthetic pathway for the chromophore. The present invention is useful for developing pharmaceutical and treating diseases such as cancer or bacterial infections.

### FIELD OF THE INVENTION

The present invention provides a chromoprotein produced by Actinomadura sp. 21G792, as well as amino acid and nucleic acid sequences of the apoprotein component of the chromoprotein and of components of the biosynthetic pathway for the chromophore. The present invention is useful for developing pharmaceutical compositions and treating diseases such as cancer or bacterial infections.

### BACKGROUND OF THE INVENTION

Enediynes, a potent class of cytotoxic polyketides produced by members of the Actinomycetales, have been used to treat cancer. The typical mode of action of the enediyne drugs is through single- and double-strand DNA cleavage. DNA cleavage is induced by hydrogen abstraction from the deoxyribose sugar backbone by a diradical generated from a Bergman-type cycloaromatization of the enediyne ring. Two enediynes are currently approved for the clinical treatment of cancer: calicheamicin conjugated to a CD33 monoclonal antibody (Mylotarg®, USA) and poly(styrene-co-maleic acid)-conjugated neocarzinostatin (Japan).

Enediyne natural products can be divided into two sub-categories. The first sub-class is characterized by a bicyclo[7,3,0]dodecadiyne (i.e., nine-membered) enediyne core or its precursor, and the second sub-class is characterized by a bicylco[7,3,1]tridecadiyne (i.e., ten-membered) enediyne core. Examples of the nine-membered enediynes include neocarzinostatin, C-1027, kedarcidin, macromomycin, N1999A2 and maduropeptin. Examples of the ten-membered sub-class include calicheamicin, esperamicin, dynemicin and namenamicin. An additional characteristic that distinguishes the nine-membered from the ten-membered enediynes is that with the exception of N1999A2, all nine-membered enediynes are produced as enediyne-protein complexes, wherein the enediyne chromophore is attached to an inactive apoprotein by non-covalent binding. For this reason the nine-membered enediynes are often referred to as chromoproteins. It is believed that the apoprotein plays the critical role of stabilizing the labile nine-membered enediyne chromophore and providing the targeted delivery of the cytotoxic chromophore to the chromatin.

The amino acid sequences of several apoproteins have been determined by directly sequencing the apoprotein or by deducing the amino acid from a cloned DNA sequence. The apoproteins identified to date are small, acidic proteins (108-114 amino acids, aa), which are generated from a pre-apoprotein by the removal of a 32-34 aa amino-terminal leader peptide. The biosynthetic pathways for two chromoproteins (neocarzinostatin and C-1027) have been cloned and sequenced. In these cases, the gene encoding the apoprotein was clustered with the genes required for the biosynthesis of the associated chromophore.

The apoprotein component of the chromoprotein complex presents an attractive target for the directed alteration of drug properties. For example, if the apoprotein amino acid or nucleic acid sequence is discovered, the chromophore-binding motif of the apoprotein can be altered using established molecular biology techniques, such as site-directed mutagenesis, to create a rationally altered apoprotein that binds its natural chromophore more strongly or weakly. Moreover, such alterations to the apoprotein could lead to, for example, a chromoprotein having decreased toxicity, or a chromophore having increased potency or stability. Additionally, extensive manipulation of the apoprotein could lead to an apoprotein with greatly altered binding specificities and, thus, the ability to function as a targeted drug delivery vehicle for molecules very different from the enediyne chromophore.

Accordingly, there exists a need for novel chromoproteins, and for isolation and characterization of the genes and proteins involved in their synthesis.

### SUMMARY OF THE INVENTION

The present invention relates to a novel highly potent anti-cancer chromoprotein produced by a terrestrial actinomycete, Actinomadura sp. 21G792 (NRRL 30778). The Actinomadura sp. 21G792 chromoprotein is a non-covalent complex of an apoprotein and a chromophore comprising a nine-membered enediyne. The chromoprotein appears to be less toxic than compounds belonging to ten-membered enediynes, presumably because of the activity-modulating effect of the apoprotein.

The present invention provides polypeptides and isolated nucleic acids encoding polypeptides of the chromoprotein biosynthetic gene cluster of Actinomadura sp. 21G792. Included among the polypeptides are components of the chromophore biosynthetic pathway and the pre-apoprotein. In a host, the apoprotein component is formed by cleavage of a signal peptide from the pre-apoprotein. Accordingly, the invention further provides nucleic acid sequences encoding the Actinomadura sp. 21G792 apoprotein fused at its N-terminal to a secretion signal peptide.

In an embodiment of the invention, the nucleic acid encodes a polypeptide having having at least about 70% homology with the amino acid sequence of SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ ID NO:20, SEQ ID NO:22, SEQ ID NO:24, SEQ ID NO:26, SEQ ID NO:28, SEQ ID NO:30, SEQ ID NO:32, SEQ ID NO:34, SEQ ID NO:36, SEQ ID NO:38, SEQ ID NO:40, SEQ ID NO:42, SEQ ID NO:44, SEQ ID NO:46, SEQ ID NO:48, SEQ ID NO:50, SEQ ID NO:52, SEQ ID NO:54, SEQ ID NO:56, SEQ ID NO:58, SEQ ID NO:60, SEQ ID NO:62, SEQ ID NO:64, SEQ ID NO:66, SEQ ID NO:68, SEQ ID NO:70, SEQ ID NO:72, SEQ ID NO:74, SEQ ID NO:76, SEQ ID NO:78, SEQ ID NO:80, SEQ ID NO:82, SEQ ID NO:84, SEQ ID NO:86, SEQ ID NO:88, SEQ ID NO:90, SEQ ID NO:92, SEQ ID NO:94, SEQ ID NO:96, SEQ ID NO:98, SEQ ID NO:100, SEQ ID NO:102, SEQ ID NO:104, SEQ ID NO:106, SEQ ID NO:108, SEQ ID NO:110, SEQ ID NO:112, SEQ ID NO:114, SEQ ID NO:116, SEQ ID NO:118, SEQ ID NO:120, SEQ ID NO:122, SEQ ID NO:124, SEQ ID NO:126, SEQ ID NO:128, SEQ ID NO:130, SEQ ID NO:132, SEQ ID NO:134, SEQ ID NO:136, SEQ ID NO:138, SEQ ID NO:140, SEQ ID NO:142, SEQ ID NO:144, SEQ ID NO:146, SEQ ID NO:148 or SEQ ID NO:150. In other embodiments of the invention, the homology may be at least about 80%, or at least about 90%, or the homology may be 100%. In certain embodiments, the sequence of the polypeptide is identical to one of SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ ID NO:20, SEQ ID NO:22, SEQ ID NO:24, SEQ ID NO:26, SEQ ID NO:28, SEQ ID NO:30, SEQ ID NO:32, SEQ ID NO:34, SEQ ID NO:36, SEQ ID NO:38, SEQ ID NO:40, SEQ ID NO:42, SEQ ID NO:44, SEQ ID NO:46, SEQ ID NO:48, SEQ ID NO:50, SEQ ID NO:52, SEQ ID NO:54, SEQ ID NO:56, SEQ ID NO:58, SEQ ID NO:60, SEQ ID NO:62, SEQ ID NO:64, SEQ ID NO:66, SEQ ID NO:68, SEQ ID NO:70, SEQ ID NO:72, SEQ ID NO:74, SEQ ID NO:76, SEQ ID NO:78, SEQ ID NO:80, SEQ ID NO:82, SEQ ID NO:84, SEQ ID NO:86, SEQ ID NO:88, SEQ ID NO:90, SEQ ID NO:92, SEQ ID NO:94, SEQ ID NO:96, SEQ ID NO:98, SEQ ID NO:100, SEQ ID NO:102, SEQ ID NO:104, SEQ ID NO:106, SEQ ID NO:108, SEQ ID NO:110, SEQ ID NO:112, SEQ ID NO:114, SEQ ID NO:116, SEQ ID NO:118, SEQ ID NO:120, SEQ ID NO:122, SEQ ID NO:124, SEQ ID NO:126, SEQ ID NO:128, SEQ ID NO:130, SEQ ID NO:132, SEQ ID NO:134, SEQ ID NO:136, SEQ ID NO:138, SEQ ID NO:140, SEQ ID NO:142, SEQ ID NO:144, SEQ ID NO:146, SEQ ID NO:148 or SEQ ID NO:150.

In certain embodiments, the nucleic acid comprises a nucleotide sequence that is at least about 70%, at least about 80%, at least about 90%, or identical to the sequence of SEQ ID NO:1, SEQ ID NO:3, SEQ ID NO:5, SEQ ID NO:7, SEQ ID NO:9, SEQ ID NO:11, SEQ ID NO:13, SEQ ID NO:15, SEQ ID NO:17, SEQ ID NO:19, SEQ ID NO:21, SEQ ID NO:23, SEQ ID NO:25, SEQ ID NO:27, SEQ ID NO:29, SEQ ID NO:31, SEQ ID NO:33, SEQ ID NO:35, SEQ ID NO:37, SEQ ID NO:39, SEQ ID NO:41, SEQ ID NO:43, SEQ ID NO:45, SEQ ID NO:47, SEQ ID NO:49, SEQ ID NO:51, SEQ ID NO:53, SEQ ID NO:55, SEQ ID NO:57, SEQ ID NO:59, SEQ ID NO:61, SEQ ID NO:63, SEQ ID NO:65, SEQ ID NO:67, SEQ ID NO:69, SEQ ID NO:71, SEQ ID NO:73, SEQ ID NO:75, SEQ ID NO:77, SEQ ID NO:79, SEQ ID NO:81, SEQ ID NO:83, SEQ ID NO:85, SEQ ID NO:87, SEQ ID NO:89, SEQ ID NO:91, SEQ ID NO:93, SEQ ID NO:95, SEQ ID NO:97, SEQ ID NO:99, SEQ ID NO:101, SEQ ID NO:103, SEQ ID NO:105, SEQ ID NO:107, SEQ ID NO:109, SEQ ID NO:111, SEQ ID NO:113, SEQ ID NO:115, SEQ ID NO:117, SEQ ID NO:119, SEQ ID NO:121, SEQ ID NO:123, SEQ ID NO:125, SEQ ID NO:127, SEQ ID NO:129, SEQ ID NO:131, SEQ ID NO:133, SEQ ID NO:135, SEQ ID NO:137, SEQ ID NO:139, SEQ ID NO:141, SEQ ID NO:143, SEQ ID NO:145, SEQ ID NO:147, or SEQ ID NO:149, or the complement thereof.

The invention also provides vectors and host cells comprising the nucleic acids. In one embodiment, the invention provides a cosmid containing DNA isolated from Actinomadura sp. 21G792, that contains all or part of the chromoprotein gene cluster. Methods for isolation and manipulation of the nucleic acids are provided. Also provided are probes and primers for identification and amplification of chromoprotein gene cluster nucleic acids.

The invention provides an isolated protein or polypeptide comprising an amino acid sequence having at least about 70% homology, at least about 80% homology, at least about 90% homology, or about 100% homology with the amino acid sequence of SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ ID NO:20, SEQ ID NO:22, SEQ ID NO:24, SEQ ID NO:26, SEQ ID NO:28, SEQ ID NO:30, SEQ ID NO:32, SEQ ID NO:34, SEQ ID NO:36, SEQ ID NO:38, SEQ ID NO:40, SEQ ID NO:42, SEQ ID NO:44, SEQ ID NO:46, SEQ ID NO:48, SEQ ID NO:50, SEQ ID NO:52, SEQ ID NO:54, SEQ ID NO:56, SEQ ID NO:58, SEQ ID NO:60, SEQ ID NO:62, SEQ ID NO:64, SEQ ID NO:66, SEQ ID NO:68, SEQ ID NO:70, SEQ ID NO:72, SEQ ID NO:74, SEQ ID NO:76, SEQ ID NO:78, SEQ ID NO:80, SEQ ID NO:82, SEQ ID NO:84, SEQ ID NO:86, SEQ ID NO:88, SEQ ID NO:90, SEQ ID NO:92, SEQ ID NO:94, SEQ ID NO:96, SEQ ID NO:98, SEQ ID NO:100, SEQ ID NO:102, SEQ ID NO:104, SEQ ID NO:106, SEQ ID NO:108, SEQ ID NO:100, SEQ ID NO:112, SEQ ID NO:114, SEQ ID NO:116, SEQ ID NO:118, SEQ ID NO:120, SEQ ID NO:122, SEQ ID NO:124, SEQ ID NO:126, SEQ ID NO:128, SEQ ID NO:130, SEQ ID NO:132, SEQ ID NO:134, SEQ ID NO:136, SEQ ID NO:138, SEQ ID NO:140, SEQ ID NO:142, SEQ ID NO:144, SEQ ID NO:146, SEQ ID NO:148, or SEQ ID NO:150, and variants thereof.

The present invention contemplates a method for producing a recombinant apoprotein; the method comprises the steps of: a) culturing a host cell which contains an expression vector having a nucleic acid sequence comprising SEQ ID NO:63 or SEQ ID NO:149 in a culture medium under conditions suitable for expression of the recombinant protein in the host cell, and b) isolating the recombinant protein from the host cell or the culture medium.

Also contemplated is a method of producing a recombinant chromoprotein. The method comprises: a) culturing a host cell which contains a cosmid or other expression vector which expresses genes encoding structural and enzymatic components (e.g., including all or a subset of orfs 1-65), and b) isolating the recombinant protein from the host cell or culture medium. The recombinant chromoprotein can be the 21G792 chromoprotein, or a variant thereof.

The present invention contemplates methods for using a nucleic acid molecule that hybridizes to or comprises a portion of SEQ ID NO:1, SEQ ID NO:3, SEQ ID NO:5, SEQ ID NO:7, SEQ ID NO:9, SEQ ID NO:11, SEQ ID NO:13, SEQ ID NO:15, SEQ ID NO:17, SEQ ID NO:19, SEQ ID NO:21, SEQ ID NO:23, SEQ ID NO:25, SEQ ID NO:27, SEQ ID NO:29, SEQ ID NO:31, SEQ ID NO:33, SEQ ID NO:35, SEQ ID NO:37, SEQ ID NO:39, SEQ ID NO:41, SEQ ID NO:43, SEQ ID NO:45, SEQ ID NO:47, SEQ ID NO:49, SEQ ID NO:51, SEQ ID NO:53, SEQ ID NO:55, SEQ ID NO:57, SEQ ID NO:59, SEQ ID NO:61, SEQ ID NO:63, SEQ ID NO:65, SEQ ID NO:67, SEQ ID NO:69, SEQ ID NO:71, SEQ ID NO:73, SEQ ID NO:75, SEQ ID NO:77, SEQ ID NO:79, SEQ ID NO:81, SEQ ID NO:83, SEQ ID NO:85, SEQ ID NO:87, SEQ ID NO:89, SEQ ID NO:91, SEQ ID NO:93, SEQ ID NO:95, SEQ ID NO:97, SEQ ID NO:99, SEQ ID NO:101, SEQ ID NO:103, SEQ ID NO:105, SEQ ID NO:107, SEQ ID NO:109, SEQ ID NO:111, SEQ ID NO:113, SEQ ID NO:115, SEQ ID NO:117, SEQ ID NO:119, SEQ ID NO:121, SEQ ID NO:123, SEQ ID NO:125, SEQ ID NO:127, SEQ ID NO:129, SEQ ID NO:131, SEQ ID NO:133, SEQ ID NO:135, SEQ ID NO:137, SEQ ID NO:139, SEQ ID NO:141, SEQ ID NO:143, SEQ ID NO:145, SEQ ID NO:147, or SEQ ID NO:149 as a probe to, for example, identify other organisms capable of producing enediyne-related compounds or to identify the genes involved in the synthesis of chromoproteins in, for example, organisms capable of producing enediyne related compounds, such as Actinomadura sp. 21G792.

The invention provides the Actinomadura sp. 21G792 apoprotein and provides substantially pure forms of the apoprotein and chromoprotein, as well as pharmaceutical compositions comprising the chromoprotein and methods for administering the chromoprotein. The chromoprotein is demonstrated to be useful for treatment of cancerous cells and tumors.

The present invention further provides a method for generating variants of the Actinomadura sp. 21G792 apoprotein that have altered biological activity. Such variant apoproteins can have altered chromophore binding properties, altered target specificity, or a combination thereof.

It will be understood that the present invention provides for production of large quantities of the apoprotein and the chromoprotein. It further will be appreciated that the invention may lead to the identification of other organisms capable of producing enediyne-related compounds or the identification of the genes involved in the synthesis of chromoproteins in, for example, organisms capable of producing enediyne related compounds, such as Actinomadura sp. 21G792. Additionally, it will be appreciated that the invention provides for the production of modified versions of the apoprotein which, for example, have decreased toxicity, increased potency, or increased stability. It also will be understood that manipulation of the Actinomadura sp. 21G792 apoprotein can lead to an apoprotein with altered binding specificities and, thus, the ability to function as a targeted drug delivery vehicle for chromophores different from the 21G792 enediyne chromophore. Finally, it will be appreciated that pharmaceutical compositions comprising the Actinoinadura sp. 21G792 chromoprotein can be developed and administered to mammals, preferably humans, having bacterial infections or cancerous growths.

### BRIEF DESCRIPTION OF THE DRAWINGS

FIG. 1 is an HPLC chromatogram of the Actinomadura sp. 21G792 chromoprotein. The analytical conditions of the HPLC were as follows. Column: TosoHaas DEAE 5 PW (10 um particle size, 7.5 mm×7.5 cm in size). Buffer: 0-0.5 M linear gradient NaCl with constant 0.05 M Tris-HCl in 25 min at a flow rate of 0.8 ml/min.

FIG. 2 is a UV spectrum of the Actinomadura sp. 21G792 chromoprotein.

FIG. 3 is an HPLC chromatogram of the 21G792 apoprotein. The analytical conditions of the HPLC were as follows. Column: VYDAC Protein C4 (300 A, 3.0×100 mm in size). Solvent: 10-30% Acetonitrile in H₂O with constant 0.05% TFA in 6 minutes at 2 ml/min.

FIG. 4 is a UV spectrum of the 21G792 chromoprotein.

FIG. 5 shows a molecular weight determination for the apoprotein (12.92409 kDa by MALDI-MS).

FIG. 6 provides the nucleotide sequence and deduced amino acid sequence of the 21G792 pre-apoprotein and apoprotein. The putative ribosome binding site is boxed, and the leader peptide is underlined. The slash mark indicates the cleavage site for leader peptide and apoprotein.

FIG. 7 depicts the open reading frames of Actinomadura sp. 21G792 chromoprotein gene cluster. Genes located on cosmid 41417 are indicated by a solid line above the orf arrows. Those located on cosmid 21gD are indicated by the dashed line composed of small dashes, and those located on cosmid 21gB are indicated by the dashed line composed of large dashes. Locations of probes used to identify each cosmid are indicated by black barbells. PstI (P) and EcoRI (E) restriction sites are labeled.

FIG. 8 depicts the structure of the Actinoinadura sp. 21G792 chromophore.

FIG. 9 depicts a pathway for synthesis of the tyrosine-derived component (3-[2-chloro-3-hydroxy-4-methoxy-phenyl]-3-hydroxy-propionic acid) of the Actinomadura sp. 21G792 chromophore.

FIG. 10 depicts structural domains of the orf17 gene product. Core motifs of the condensation (C), adenylation (A) and peptidyl-carrier protein (PCP) domains are boxed and labeled. Residues contributing to the A domain substrate specificity code for the orf17 gene product and SgcC4 of the C-1027 biosynthetic pathway are in bold and underlined. Identical residues are marked with an asterisk, a colon indicates conserved residues and a semi-colon indicates semi-conserved residues.

FIG. 11 depicts a pathway for synthesis of the madurosamine (4-amino-4-deoxy-3-C-methyl-β-ribopyranose) component of the Actinomadura sp. 21G792 chromophore.

FIG. 12 depicts the alignment of Orf38 with dNDP-glucose-4,6-dehydratases and UDP-glucuronate decarboxylases. Glucose-4,6-dehydratase sequences included in the alignment are Orf5 from the Streptomyces neyagawaensis concanamycin A gene cluster (AAZ94396), MtmE from the Streptomyces argillaceus mithramycin gene cluster (CAA71847), and SpcE from the Streptomyces spectabilis spectinomycin gene cluster (AAD31797). Glucuronate decarboxylase sequences included in the alignment are Uxs1 from Pisum sativum (BAB40967), Uxs3 from Arabidopsis thaliana (AAK70882), Uxs1 from Arabidopsis thaliana (AAK70880), Uxs2 from Arabidopsis thaliana (AAK70881), Uxs1 from Mus musculus (AAK85410) and Uxs1 from Cryptococcus neoformans (AAK59981). Identical residues are marked with an asterisk, a colon indicates conserved residues and a semi-colon indicates semi-conserved residues.

FIG. 13 depicts a pathway for synthesis of the 2-hydroxy-3,6-dimethyl benzoic acid component of the Actinomadura sp. 21G792 chromophore.

FIG. 14 depicts the alignment of the region between the A4 and A5 core motifs of Orf31 and ten aryl acid-AMP ligases. Structural anchors are shaded in black. Proposed constituents of the carboxy acid binding pockets are shaded in grey. Residues proposed to be involved in discrimination between the activation of DHBA and salicylic acid are identified with a number sign. Identical residues are marked with an asterisk, a colon indicates conserved residues and a semi-colon indicates semi-conserved residues.

FIG. 15 depicts a biosynthetic pathway for the generation of the enediyne core of the Actinomadura sp. 21G792 chromophore.

FIG. 16 depicts the domain organization and comparison of Orf5 with the SgcE and NcsE enediyne PKSs. aa, amino acid; KS, ketosynthase; AT, acetyltransferase; ACP, acyl carrier protein; KR, ketoreductase; DH, dehydratase; TD, terminal domain.

FIG. 17 depicts a route to assembly of the four components of the Actinomadura sp. 21G792 chromophore.

FIG. 18 is a graph demonstrating that the 21G792 chromoprotein induced dose-dependent DNA strand breaks occur in p21-proficient and p21-deficient HCT116 human colon carcinoma cells at >100 ng/ml chromoprotein concentrations.

FIG. 19 is a DNA cleavage assay showing that the 21G792 chromoprotein induced single strand breaks and double strand breaks, the reaction continued to progress over 24 hours, and DNA cleavage did not require a thiol agent.

FIG. 20 depicts digestion of Histone H1 by the Actinomadura sp. 21G792 chromoprotein and inhibition by DNA. Protease inhibitors are PMSF, Leupeptin, Aprotinin, and Pepstatin A. The apoprotein has no activity.

FIG. 21 depicts relative sensitivity of histones H1, H2A, H₂B, H3, and H4 to digestion by the Actinomadura sp. 21G792 chromoprotein. Basic proteins such as myelin basic protein, but not neutral/acidic proteins, are also susceptible to cleavage.

FIG. 22 depicts histone H1 reduction in cells treated with the Actinomadura sp. 21G792 chromoprotein, but not bleomycin or calicheamicin.

FIG. 23A is a protein immunoblot showing that exposure of HCT116 cells to the chromoprotein at various concentrations results in the activation of the p53/p21 checkpoint. FIG. 23B depicts phosphorylation of the serine-15 amino acid residue of p53 at the cleavage of poly-ADP-ribose phosphorylase (ParP).

FIGS. 24 and 25 are a series of graphs showing the in vivo potency of the 21G792 chromoprotein against tumors of subcutaneously injected LoVo (colon cancer); HCT116 (colon); HT29 (colon); LOX (melanoma); HN5 (head & neck); and PC-3 (prostate) cells in athymic (nude) mice.

FIG. 26 depicts uptake of FITC labeled Actinomadura sp. 21G792 chromoprotein by HCT116 cells.

FIG. 27 depicts uptake of FITC labeled Actinomadura sp. 21G792 chromoprotein and apoprotein by HCT116 cells.

FIG. 28 depicts uptake of labeled Actinomadura sp. 21G792 chromoprotein in the presence of a 10 fold greater concentration of unlabeled chromoprotein.

FIG. 29 depicts the effect of an energy uncoupling agent (sodium azide) or a tubulin disrupting agent (nocodazole) on uptake of the Actinomadura sp. 21G792 apoprotein by HCT116 cells.

FIG. 30 depicts linkage of a monoclonal antibody to a derivative of the Actinomadura sp. 21G792 chromophore.

### DETAILED DESCRIPTION OF THE INVENTION

Enediyne antibiotics are produced by a variety of organisms generally belonging to the order Actinomycetales, including but not limited to the genera Streptomyces, Micromonospora, and Actinomadura. The present invention relates to a novel chromoprotein produced by Actinomadura sp. 21G792, deposited at the Agricultural Research Service Culture Collection (NRRL, 1815 North University Street, Peoria, Ill., 61064). The deposits were made under the terms of the Budapest Treaty. Actinomadura sp. 21G792 has been given accession number NRRL 30778. Of such organisms known to date, Actinomadura sp. 21G792 appears to be most similar to the Actinomadura strain deposited as ATCC 39144 (U.S. Pat. No. 4,546,084). As assessed by 16S rDNA sequences, the strains are related species or subspecies.

The Actinomadura sp. 21G792 chromoprotein consists of a novel apoprotein and chromophore. Components of the chromoprotein and of the chromophore biosynthetic pathway, or precursors of those components (i.e., the pre-apoprotein), are encoded by a contiguous set of open reading frames (orfs) referred to as the chromoprotein biosynthetic gene cluster. Accordingly, the invention provides an isolated nucleic acid that encodes an orf of the Actinomadura sp. 21G792 chromoprotein biosynthetic gene cluster (See Table 1), or an expressed (i.e., processed) fragment thereof (e.g., an apoprotein; SEQ ID NO:150). In one embodiment, the invention provides a nucleic acid having a nucleotide sequence that encodes the amino acid sequence of SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ ID NO:20, SEQ ID NO:22, SEQ ID NO:24, SEQ ID NO:26, SEQ ID NO:28, SEQ ID NO:30, SEQ ID NO:32, SEQ ID NO:34, SEQ ID NO:36, SEQ ID NO:38, SEQ ID NO:40, SEQ ID NO:42, SEQ ID NO:44, SEQ ID NO:46, SEQ ID NO:48, SEQ ID NO:50, SEQ ID NO:52, SEQ ID NO:54, SEQ ID NO:56, SEQ ID NO:58, SEQ ID NO:60, SEQ ID NO:62, SEQ ID NO:64, SEQ ID NO:66, SEQ ID NO:68, SEQ ID NO:70, SEQ ID NO:72, SEQ ID NO:74, SEQ ID NO:76, SEQ ID NO:78, SEQ ID NO:80, SEQ ID NO:82, SEQ ID NO:84, SEQ ID NO:86, SEQ ID NO:88, SEQ ID NO:90, SEQ ID NO:92, SEQ ID NO:94, SEQ ID NO:96, SEQ ID NO:98, SEQ ID NO:100, SEQ ID NO:102, SEQ ID NO:104, SEQ ID NO:106, SEQ ID NO:108, SEQ ID NO:110, SEQ ID NO:112, SEQ ID NO:114, SEQ ID NO:116, SEQ ID NO:118, SEQ ID NO:120, SEQ ID NO:122, SEQ ID NO:124, SEQ ID NO:126, SEQ ID NO:128, SEQ ID NO:130, SEQ ID NO:132, SEQ ID NO:134, SEQ ID NO:136, SEQ ID NO:138, SEQ ID NO:140, SEQ ID NO:142, SEQ ID NO:144, SEQ ID NO:146, SEQ ID NO:148, or SEQ ID NO:150. In a preferred embodiment, the nucleic acids comprise the nucleotide sequence of SEQ ID NO:1, SEQ ID NO:3, SEQ ID NO:5, SEQ ID NO:7, SEQ ID NO:9, SEQ ID NO:11, SEQ ID NO:13, SEQ ID NO:15, SEQ ID NO:17, SEQ ID NO:19, SEQ ID NO:21, SEQ ID NO:23, SEQ ID NO:25, SEQ ID NO:27, SEQ ID NO:29, SEQ ID NO:31, SEQ ID NO:33, SEQ ID NO:35, SEQ ID NO:37, SEQ ID NO:39, SEQ ID NO:41, SEQ ID NO:43, SEQ ID NO:45, SEQ ID NO:47, SEQ ID NO:49, SEQ ID NO:51, SEQ ID NO:53, SEQ ID NO:55, SEQ ID NO:57, SEQ ID NO:59, SEQ ID NO:61, SEQ ID NO:63, SEQ ID NO:65, SEQ ID NO:67, SEQ ID NO:69, SEQ ID NO:71, SEQ ID NO:73, SEQ ID NO:75, SEQ ID NO:77, SEQ ID NO:79, SEQ ID NO:81, SEQ ID NO:83, SEQ ID NO:85, SEQ ID NO:87, SEQ ID NO:89, SEQ ID NO:91, SEQ ID NO:93, SEQ ID NO:95, SEQ ID NO:97, SEQ ID NO:99, SEQ ID NO:101, SEQ ID NO:103, SEQ ID NO:105, SEQ ID NO:107, SEQ ID NO:109, SEQ ID NO:111, SEQ ID NO:113, SEQ ID NO:115, SEQ ID NO:117, SEQ ID NO:119, SEQ ID NO:121, SEQ ID NO:123, SEQ ID NO:125, SEQ ID NO:127, SEQ ID NO:129, SEQ ID NO:131, SEQ ID NO:133, SEQ ID NO:135, SEQ ID NO:137, SEQ ID NO:139, SEQ ID NO:141, SEQ ID NO:143, SEQ ID NO:145, SEQ ID NO:147, or SEQ ID NO:149. It will be appreciated that the nucleic acids of the invention include complementary sequences.

TABLE 1
Open Reading Frames of the 21G792 Chromoprotein Gene Cluster

| | Start/Stop | SEQ | Length | SEQ |
|---|---|---|---|---|
| Orf | (bp) | ID NO | (aa) | ID NO |
| 9* | Start/1391 | 1 | incomplete | 2 |
| 8* | 1475/1861 | 3 | 128 | 4 |
| 7* | 1916/2371 | 5 | 151 | 6 |
| 6* | 2672/4270 | 7 | 532 | 8 |
| 5* | 4984/4349 | 9 | 211 | 10 |
| 4* | 5054/6631 | 11 | 525 | 12 |
| 3* | 6685/6891 | 13 | 68 | 14 |
| 2* | 7472/6984 | 15 | 162 | 16 |
| 1* | 8971/7475 | 17 | 498 | 18 |
| 1 | 9268/10263 | 19 | 331 | 20 |
| 2 | 10592/ | 21 | 300 | 22 |
| | 11494 | | | |
| 3 | 11498/ | 23 | 678 | 24 |
| | 13534 | | | |
| 4 | 13541/ | 25 | 330 | 26 |
| | 14533 | | | |
| 5 | 14530/ | 27 | 1944 | 28 |
| | 20364 | | | |
| 6 | 20369/ | 29 | 152 | 30 |
| | 20827 | | | |
| 7 | 20824/ | 31 | 183 | 32 |
| | 21375 | | | |
| 8 | 21372/ | 33 | 464 | 34 |
| | 22766 | | | |
| 9 | 23607/ | 35 | 251 | 36 |
| | 22852 | | | |
| 10 | 24877/ | 37 | 336 | 38 |
| | 23867 | | | |
| 11 | 25277/ | 39 | 218 | 40 |
| | 25933 | | | |
| 12 | 25930/ | 41 | 552 | 42 |
| | 27588 | | | |
| 13 | 27602/ | 43 | 365 | 44 |
| | 28699 | | | |
| 14 | 28792/ | 45 | 261 | 46 |
| | 29577 | | | |
| 15 | 29591/ | 47 | 229 | 48 |
| | 30280 | | | |
| 16 | 30631/ | 49 | 95 | 50 |
| | 30344 | | | |
| 17 | 30845/ | 51 | 1120 | 52 |
| | 34207 | | | |
| 18 | 34204/ | 53 | 537 | 54 |
| | 35817 | | | |
| 19 | 35852/ | 55 | 548 | 56 |
| | 37498 | | | |
| 20 | 37516/ | 57 | 460 | 58 |
| | 38898 | | | |
| 21 | 39250/ | 59 | 442 | 60 |
| | 40578 | | | |
| 22 | 40705/ | 61 | 525 | 62 |
| | 42282 | | | |
| 23 | 43151/ | 63 | 165 | 64 |
| | 42654 | | | |
| 24 | 43376/ | 65 | 461 | 66 |
| | 44761 | | | |
| 25 | 44805/ | 67 | 408 | 68 |
| | 46031 | | | |
| 26 | 46045/ | 69 | 381 | 70 |
| | 47190 | | | |
| 27 | 47187/ | 71 | 409 | 72 |
| | 48416 | | | |
| 28 | 49128/ | 73 | 232 | 74 |
| | 48430 | | | |
| 29 | 49328/ | 75 | 466 | 76 |
| | 50728 | | | |
| 30 | 50725/ | 77 | 285 | 78 |
| | 51582 | | | |
| 31 | 53282/ | 79 | 548 | 80 |
| | 51636 | | | |
| 32 | 58519/ | 81 | 1746 | 82 |
| | 53279 | | | |
| 33 | 59639/ | 83 | 348 | 84 |
| | 58593 | | | |
| 34 | 59897/ | 85 | 393 | 86 |
| | 61078 | | | |
| 35 | 61119/ | 87 | 148 | 88 |
| | 61565 | | | |
| 36 | 61568/ | 89 | 401 | 90 |
| | 62773 | | | |
| 37 | 62785/ | 91 | 447 | 92 |
| | 64128 | | | |
| 38 | 64131/ | 93 | 328 | 94 |
| | 65117 | | | |
| 39 | 65134/ | 95 | 539 | 96 |
| | 66753 | | | |
| 40 | 68054/ | 97 | 406 | 98 |
| | 66834 | | | |
| 41 | 68270/ | 99 | 340 | 100 |
| | 69292 | | | |
| 42 | 69375/ | 101 | 460 | 102 |
| | 70757 | | | |
| 43 | 71889/ | 103 | 347 | 104 |
| | 70846 | | | |
| 44 | 72452/ | 105 | 138 | 106 |
| | 72036 | | | |
| 45 | 72706/ | 107 | 557 | 108 |
| | 74379 | | | |
| 46 | 75114/ | 109 | 230 | 110 |
| | 74422 | | | |
| 47 | 75189/ | 111 | 403 | 112 |
| | 76400 | | | |
| 48 | 77794/ | 113 | 444 | 114 |
| | 76460 | | | |
| 49 | 78801/ | 115 | 277 | 116 |
| | 77968 | | | |
| 50 | 78892/ | 117 | 213 | 118 |
| | 79533 | | | |
| 51 | 80344/ | 119 | 266 | 120 |
| | 79544 | | | |
| 52 | 80936/ | 121 | 196 | 122 |
| | 80346 | | | |
| 53 | 81022/ | 123 | 109 | 124 |
| | 81351 | | | |
| 54 | 81348/ | 125 | 142 | 126 |
| | 81776 | | | |
| 55 | 82077/ | 127 | 292 | 128 |
| | 82955 | | | |
| 56 | 82998/ | 129 | 337 | 130 |
| | 84011 | | | |
| 57 | 84224/ | 131 | 352 | 132 |
| | 85282 | | | |
| 58 | 85643/ | 133 | 69 | 134 |
| | 85434 | | | |
| 59 | 87546/ | 135 | 592 | 136 |
| | 85768 | | | |
| 60 | 87826/ | 137 | 59 | 138 |
| | 87647 | | | |
| 61 | 87909/ | 139 | 25 | 140 |
| | 87832 | | | |
| 62 | 88485/ | 141 | 167 | 142 |
| | 87982 | | | |
| 63 | 88571/ | 143 | 259 | 144 |
| | 89350 | | | |
| 64 | 89542/ | 145 | 144 | 146 |
| | 89976 | | | |
| 65 | End [90573]/ | 147 | incomplete | 148 |
| | 89980 | | | |

*involved in primary metabolism

The invention provides nucleic acids that specifically hybridize (or specifically bind) under stringent hybridization conditions to SEQ ID NO:1, SEQ ID NO:3, SEQ ID NO:5, SEQ ID NO:7, SEQ ID NO:9, SEQ ID NO:11, SEQ ID NO:13, SEQ ID NO:15, SEQ ID NO:17, SEQ ID NO:19, SEQ ID NO:21, SEQ ID NO:23, SEQ ID NO:25, SEQ ID NO:27, SEQ ID NO:29, SEQ ID NO:31, SEQ ID NO:33, SEQ ID NO:35, SEQ ID NO:37, SEQ ID NO:39, SEQ ID NO:41, SEQ ID NO:43, SEQ ID NO:45, SEQ ID NO:47, SEQ ID NO:49, SEQ ID NO:51, SEQ D NO:53, SEQ ID NO:55, SEQ ID NO:57, SEQ ID NO:59, SEQ ID NO:61, SEQ ID NO:63, SEQ ID NO:65, SEQ ID NO:67, SEQ ID NO:69, SEQ ID NO:71, SEQ ID NO:73, SEQ ID NO:75, SEQ ID NO:77, SEQ ID NO:79, SEQ ID NO:81, SEQ ID NO:83, SEQ ID NO:85, SEQ ID NO:87, SEQ ID NO:89, SEQ ID NO:91, SEQ ID NO:93, SEQ ID NO:95, SEQ ID NO:97, SEQ ID NO:99, SEQ ID NO:101, SEQ ID NO:103, SEQ ID NO:105, SEQ ID NO:107, SEQ ID NO:109, SEQ ID NO:11, SEQ ID NO:113, SEQ ID NO:115, SEQ ID NO:117, SEQ ID NO:119, SEQ ID NO:121, SEQ ID NO:123, SEQ ID NO:125, SEQ ID NO:127, SEQ ID NO:129, SEQ ID NO:131, SEQ ID NO:133, SEQ ID NO:135, SEQ ID NO:137, SEQ ID NO:139, SEQ ID NO:141, SEQ ID NO:143, SEQ ID NO:145, SEQ ID NO:147, or SEQ ID NO:149. Also contemplated are nucleic acids that would specifically bind to the aforementioned sequences but for the degeneracy of the nucleic acid code. The nucleic acids can be of sufficient length to encode a complete protein (e.g., a complete or D or a fragment thereof. Also included are nucleic acids that encode modified proteins. Examples of protein modifications include, but are not limited to, fusions to targeting molecules such as antibodies, antibody fragments, receptor ligands and the like.

The nucleic acids further include probes and primers. In certain embodiments, the probes or primers may be degenerate. Further, in accordance with their use, probes and primers may be single or double stranded. Probes and primers include, for example, oligonucleotides that are at least about 12 nucleotides in length, preferably at least about 15 nucleotides in length, and more preferably at least about 18 nucleotides in length, and further include PCR amplification products that might be generated using primers of the invention.

Hybridization under stringent conditions refers to conditions under which a probe will hybridize preferentially to its target subsequence, and to a lesser extent to, or not at all to, other sequences. It also will be understood that stringent hybridization and stringent hybridization wash conditions in the context of nucleic acid hybridization experiments such as southern and northern hybridizations are sequence dependent, and are different under different environmental parameters. It is well known in the art to adjust hybridization and wash solution contents and temperatures such that stringent hybridization conditions are obtained. Stringency depends on such parameters as the size and nucleotide content of the probe being utilized. See Sambrook et al., 1989, Molecular Cloning—A Laboratory Manual (2nd ed.) Vol. 1-3, Cold Spring Harbor Laboratory, Cold Spring Harbor Press, NY, and other sources for general descriptions and examples. Another guide to the hybridization of nucleic acids is found in Tijssen, 1993, Laboratory Techniques in Biochemistry and Molecular Biology—Hybridization with Nucleic Acid Probes, part I, chapter 2, Overview of principles of hybridization and the strategy of nucleic acid probe assays, Elsevier, N.Y.

Preferred stringent conditions are those that allow a probe to hybridize to a sequence that is more than about 90% complementary to the probe and not to a sequence that is less than about 70% complementary. Generally, highly stringent hybridization and wash conditions are selected to be about 5° C. lower than the thermal melting point (Tₘ) for the specific sequence at a defined ionic strength and pH. The Tₘ is the temperature (under defined ionic strength and pH) at which 50% of the target sequence hybridizes to a perfectly matched probe. Very stringent conditions are selected to be equal to the Tₘ for a particular probe.

An example of stringent hybridization conditions for hybridization of complementary nucleic acids which have more than 100 complementary residues on a filter in a Southern or northern blot is 50% formamide with 1 mg of heparin at 42° C., with the hybridization being carried out overnight. An example of highly stringent wash conditions is 0.15 M NaCl at 72° C. for about 15 minutes. An example of stringent wash conditions is a 0.2 times SSC wash at 65° C. for 15 minutes (see, Sambrook et al., 1989). Often, a high stringency wash is preceded by a low stringency wash to remove background probe signal. An example of a medium stringency wash for a duplex of, e.g., more than 100 nucleotides, is 1 times SSC at 45° C. for 15 minutes. An example of a low stringency wash for a duplex of, e.g., more than 100 nucleotides, is 4-6 times SSC at 40° C. for 15 minutes. In general, a signal to noise ratio that is two times (or higher) that observed for an unrelated probe in the particular hybridization assay indicates detection of a specific hybridization.

Nucleic acids which do not hybridize to each other under stringent conditions are still substantially identical if the polypeptides which they encode are substantially identical. This occurs, e.g., when a copy of a nucleic acid is created using the maximum codon degeneracy permitted by the genetic code. Accordingly, nucleotide sequences of the invention include sequences of nucleotides that are at least about 70%, preferably at least about 80%, and more preferably at least about 90% identical to SEQ ID NO:1, SEQ ID NO:3, SEQ ID NO:5, SEQ ID NO:7, SEQ ID NO:9, SEQ ID NO:11, SEQ ID NO:13, SEQ ID NO:15, SEQ ID NO:17, SEQ ID NO:19, SEQ ID NO:21, SEQ ID NO:23, SEQ ID NO:25, SEQ ID NO:27, SEQ ID NO:29, SEQ ID NO:31, SEQ ID NO:33, SEQ ID NO:35, SEQ ID NO:37, SEQ ID NO:39, SEQ ID NO:41, SEQ ID NO:43, SEQ ID NO:45, SEQ ID NO:47, SEQ ID NO:49, SEQ ID NO:51, SEQ ID NO:53, SEQ ID NO:55, SEQ ID NO:57, SEQ ID NO:59, SEQ ID NO:61, SEQ ID NO:63, SEQ ID NO:65, SEQ ID NO:67, SEQ ID NO:69, SEQ ID NO:71, SEQ ID NO:73, SEQ ID NO:75, SEQ ID NO:77, SEQ ID NO:79, SEQ ID NO:81, SEQ ID NO:83, SEQ ID NO:85, SEQ ID NO:87, SEQ ID NO:89, SEQ ID NO:91, SEQ ID NO:93, SEQ ID NO:95, SEQ ID NO:97, SEQ ID NO:99, SEQ ID NO:101, SEQ ID NO:103, SEQ ID NO:105, SEQ ID NO:107, SEQ ID NO:109, SEQ ID NO:111, SEQ ID NO:113, SEQ ID NO:115, SEQ ID NO:117, SEQ ID NO:119, SEQ ID NO:121, SEQ ID NO:123, SEQ ID NO:125, SEQ ID NO:127, SEQ ID NO:129, SEQ ID NO:131, SEQ ID NO:133, SEQ ID NO:135, SEQ ID NO:137, SEQ ID NO:139, SEQ ID NO:141, SEQ ID NO:143, SEQ ID NO:145, SEQ ID NO:147, or SEQ ID NO:149 or fragments thereof that are at least about 50 nucleotides, more preferably at least about 100 nucleotides in length.

The present invention is also directed to methods of producing one or more proteins encoded by the chromophore gene cluster. Such proteins may be produced by expressing one or more nucleic acids comprising SEQ ID NO:1, SEQ ID NO:3, SEQ ID NO:5, SEQ ID NO:7, SEQ ID NO:9, SEQ ID NO:11, SEQ ID NO:13, SEQ ID NO:15, SEQ ID NO:17, SEQ ID NO:19, SEQ ID NO:21, SEQ ID NO:23, SEQ ID NO:25, SEQ ID NO:27, SEQ ID NO:29, SEQ ID NO:31, SEQ ID NO:33, SEQ ID NO:35, SEQ ID NO:37, SEQ ID NO:39, SEQ ID NO:41, SEQ ID NO:43, SEQ ID NO:45, SEQ ID NO:47, SEQ ID NO:49, SEQ ID NO:51, SEQ ID NO:53, SEQ ID NO:55, SEQ ID NO:57, SEQ ID NO:59, SEQ ID NO:61, SEQ ID NO:63, SEQ ID NO:65, SEQ ID NO:67, SEQ ID NO:69, SEQ ID NO:71, SEQ ID NO:73, SEQ ID NO:75, SEQ ID NO:77, SEQ ID NO:79, SEQ ID NO:81, SEQ ID NO:83, SEQ ID NO:85, SEQ ID NO:87, SEQ ID NO:89, SEQ ID NO:91, SEQ ID NO:93, SEQ ID NO:95, SEQ ID NO:97, SEQ ID NO:99, SEQ ID NO:101, SEQ ID NO:103, SEQ ID NO:105, SEQ ID NO:107, SEQ ID NO:109, SEQ ID NO:111, SEQ ID NO:113, SEQ ID NO:115, SEQ ID NO:117, SEQ ID NO:119, SEQ ID NO:121, SEQ ID NO:123, SEQ ID NO:125, SEQ D NO:127, SEQ ID NO:129, SEQ ID NO:131, SEQ ID NO:133, SEQ ID NO:135, SEQ ID NO:137, SEQ ID NO:139, SEQ ID NO:141, SEQ ID NO:143, SEQ ID NO:145, SEQ ID NO:147, or SEQ ID NO:149 in a host cell. For example, one or more of the aforementioned nucleic acids can be operably linked to regulatory control nucleic acids to affect expression, and incorporated into a vector for expression in a host cell. In one embodiment of the invention, the apoprotein or the pre-apoprotein is produced.

Control elements useful in the present invention include promoters, optionally containing operator sequences and ribosome binding sites. Other regulatory sequences may also be desirable, such as those which allow for regulation of expression of apoprotein or pre-apoprotein relative to the growth of the host cell. Regulatory sequences are known to those of skill in the art, and examples include those which cause the expression of a gene to be turned on or off in response to a chemical or physical stimulus, including the presence of a regulatory compound. Other types of regulatory elements may also be present in the vector, for example, enhancer sequences. Various expression vectors are known in the art, e.g., cosmids, Pls, YACs, BACs, PACs, HACs.

Selectable markers can also be included in the recombinant expression vectors. A variety of markers are known which are useful in selecting for transformed cell lines and generally comprise a gene whose expression confers a selectable phenotype on transformed cells when the cells are grown in an appropriate selective medium. Such markers include, for example, genes that confer antibiotic resistance or sensitivity to the plasmid.

The vectors described above can be inserted in any prokaryotic or eukaryotic cell suitable for protein expression. Host cells include, but are not limited to Actinomadura, Streptomyces, Micrononospora, Actinomyces, Nonomurea, Pseudomonas, and the like. Preferred host cells are those of species or strains (e.g. bacterial strains) that naturally express enediynes such as Actinomadura, Streptomyces, and Micromonospora. (See, e.g., Pfeifer et al., 2001, Science 291, 1790-2; Martinez et al., 2004, Appl. Environ. Microbiol. 70, 2452-63) In one embodiment, the proteins are expressed in E. coli. Recovery of the expression products can be accomplished according to standard methods well known to those of skill in the art. Thus, for example, the proteins can be expressed with a convenient tag to facilitate isolation (e.g., a His₆ tag). Other standard protein purification techniques are suitable and well known to those of skill in the art (see, e.g., Quadri et al., 1998, Biochemistry 37, 1585-95; Nakano et al., 1992, Mol. Gen. Genet. 232, 313-21). When the entire chromoprotein gene cluster is expressed, the chromoprotein can be recovered. By selecting certain orfs for expression, chromoprotein related compounds can be produced. For example, the pre-apoprotein can be produced by expression of orf23.

One may also use a nucleic acid molecule comprising SEQ ID NO:1, SEQ ID NO:3, SEQ ID NO:5, SEQ ID NO:7, SEQ ID NO:9, SEQ ID NO:11, SEQ ID NO:13, SEQ ID NO:15, SEQ ID NO:17, SEQ ID NO:19, SEQ ID NO:21, SEQ ID NO:23, SEQ ID NO:25, SEQ ID NO:27, SEQ ID NO:29, SEQ ID NO:31, SEQ ID NO:33, SEQ ID NO:35, SEQ ID NO:37, SEQ ID NO:39, SEQ ID NO:41, SEQ ID NO:43, SEQ ID NO:45, SEQ ID NO:47, SEQ ID NO:49, SEQ ID NO:51, SEQ ID NO:53, SEQ ID NO:55, SEQ ID NO:57, SEQ ID NO:59, SEQ ID NO:61, SEQ ID NO:63, SEQ ID NO:65, SEQ ID NO:67, SEQ ID NO:69, SEQ ID NO:71, SEQ ID NO:73, SEQ ID NO:75, SEQ ID NO:77, SEQ ID NO:79, SEQ ID NO:81, SEQ ID NO:83, SEQ ID NO:85, SEQ ID NO:87, SEQ ID NO:89, SEQ ID NO:91, SEQ ID NO:93, SEQ ID NO:95, SEQ ID NO:97, SEQ ID NO:99, SEQ ID NO:101, SEQ ID NO:103, SEQ ID NO:105, SEQ ID NO:107, SEQ ID NO:109, SEQ ID NO:111, SEQ ID NO:113, SEQ ID NO:115, SEQ ID NO:117, SEQ ID NO:119, SEQ ID NO:121, SEQ ID NO:123, SEQ ID NO:125, SEQ ID NO:127, SEQ ID NO:129, SEQ ID NO:131, SEQ ID NO:133, SEQ ID NO:135, SEQ ID NO:137, SEQ ID NO:139, SEQ ID NO:141, SEQ ID NO:143, SEQ ID NO:145, SEQ ID NO:147, or SEQ ID NO:149, or a fragment thereof as a probe. Such probes are useful to identify nucleic acids of the invention. One may use the nucleotide sequences as a probe by any suitable method, including a method similar to that described in the Examples below. As described herein, a dNDP-glucose-4,6-dehydratase (DH) probe was used to identify cosmid clones of Actinomadura sp. 21G792 genomic DNA that might contain a gene or gene cluster encoding an apoprotein or other chromophore related proteins. Similarly, the nucleic acids of the invention can be used to identify orfs encoding apoproteins and chromophore related proteins, particularly nine-membered ring enediyne chromophores, in other organisms. Such organisms generally include organisms that produce secondary metabolites, such as, for example, fungi, bacillus, pseudomonads, myxobacteria and cyanobacteria. Preferably, the nucleic acids are used to identify genes of an organism of the order Actinomycetales (Taxonomic Outline of the Procaryotic Genera: Bergey's Manual® of Systematic Bacteriology, 2^{nd} Edition) including but not limited to an organism of the genus Actinomyces, Streptomyces or Micromonospora. More preferably, the nucleic acids are used to identify genes of species and subspecies of Actinomadura.

The present invention also provides substantially pure proteins and polypeptides. The term “substantially pure” as used herein in reference to a given polypeptide means that the polypeptide is substantially free from other biological macromolecules. For example, the substantially pure polypeptide is at least 75%, 80%, 85%, 95%, or 99% pure by dry weight. Purity can be measured by any appropriate standard method known in the art, for example, by column chromatography, polyacrylamide gel electrophoresis, or HPLC analysis. It will be appreciated that substantially pure proteins include chromoproteins, wherein an apoprotein is complexed with an enediyne molecule. Such attachment can be, for example, by a covalent or non-covalent bond, e.g., a hydrogen bond.

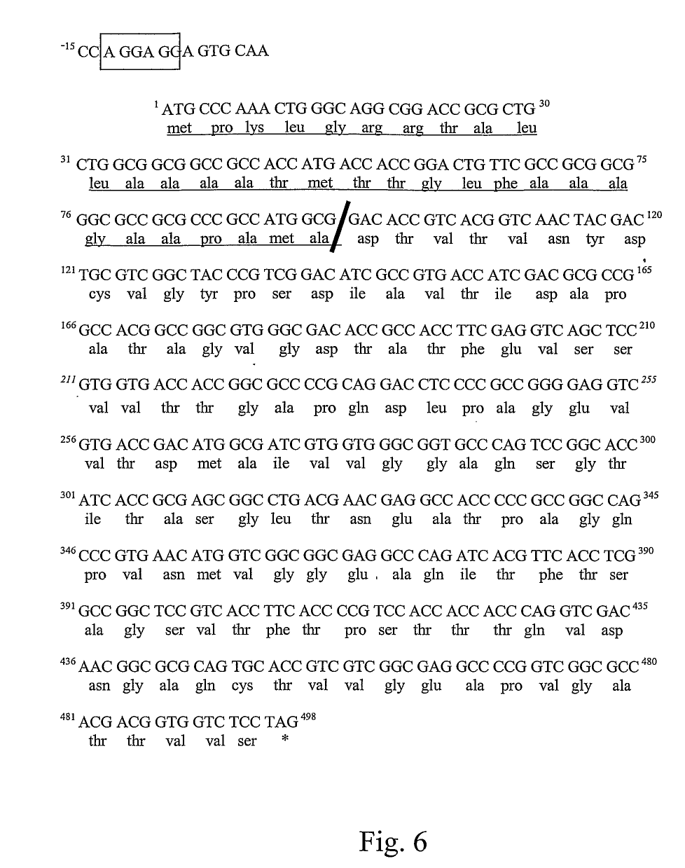

Proteins and polypeptides of the invention include those encoded by the orfs of the chromoprotein gene cluster of Actinomadura sp. 21G792. In preferred embodiments, the proteins and polypeptides are those comprising SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ ID NO:20, SEQ ID NO:22, SEQ ID NO:24, SEQ ID NO:26, SEQ ID NO:28, SEQ ID NO:30, SEQ ID NO:32, SEQ ID NO:34, SEQ ID NO:36, SEQ ID NO:38, SEQ ID NO:40, SEQ ID NO:42, SEQ ID NO:44, SEQ ID NO:46, SEQ ID NO:48, SEQ ID NO:50, SEQ ID NO:52, SEQ ID NO:54, SEQ ID NO:56, SEQ ID NO:58, SEQ ID NO:60, SEQ ID NO:62, SEQ ID NO:64, SEQ ID NO:66, SEQ ID NO:68, SEQ ID NO:70, SEQ ID NO:72, SEQ ID NO:74, SEQ ID NO:76, SEQ ID NO:78, SEQ ID NO:80, SEQ ID NO:82, SEQ ID NO:84, SEQ ID NO:86, SEQ ID NO:88, SEQ ID NO:90, SEQ ID NO:92, SEQ ID NO:94, SEQ ID NO:96, SEQ ID NO:98, SEQ ID NO:100, SEQ ID NO:102, SEQ ID NO:104, SEQ ID NO:106, SEQ ID NO:108, SEQ ID NO:110, SEQ ID NO:112, SEQ ID NO:114, SEQ ID NO:116, SEQ ID NO:118, SEQ ID NO:120, SEQ ID NO:122, SEQ ID NO:124, SEQ ID NO:126, SEQ ID NO:128, SEQ ID NO:130, SEQ ID NO:132, SEQ ID NO:134, SEQ ID NO:136, SEQ ID NO:138, SEQ ID NO:140, SEQ ID NO:142, SEQ ID NO:144, SEQ ID NO:146, SEQ ID NO:148, or SEQ ID NO:150. In a particular preferred embodiment, the protein is the 21G792 pre-apoprotein (SEQ ID NO:64) or apoprotein (SEQ ID NO:150) (FIG. 6). Amino acid compositions of the 21G792 pre-apoprotein and apoprotein are provided in Table 2.

TABLE 2
Amino Acid Composition of the
Actinomadura sp. 21G792 Apoprotein

| Amino Acid | Number | Composition (%) |
|---|---|---|
| Asp | 8 | 6.02 |
| Asn | 4 | 3.01 |
| Thr | 23 | 17.29 |
| Ser | 9 | 6.77 |
| Glu | 5 | 3.76 |
| Gln | 6 | 4.51 |
| Pro | 8 | 6.02 |
| Gly | 16 | 12.03 |
| Ala | 17 | 12.78 |
| Val | 21 | 15.79 |
| Cys | 2 | 1.50 |
| Met | 2 | 1.50 |
| Ile | 5 | 3.76 |
| Leu | 2 | 1.50 |
| Tyr | 2 | 1.50 |
| Phe | 3 | 2.26 |

It will also be appreciated that proteins or polypeptides of the invention further include those having substantially the same amino acid sequence as the aforementioned preferred proteins and polypeptides. Substantially the same amino acid sequence is defined herein as a sequence with at least about 70%, preferably at least about 80%, and more preferably at least about 90% homology, as determined by the FASTA search method in accordance with Pearson and Lipman, 1988, Proc. Natl. Acad. Sci. USA 85, 2444-8, including sequences that are at least about 70%, preferably at least about 80%, and more preferably at least about 90% identical, to SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ ID NO:20, SEQ ID NO:22, SEQ ID NO:24, SEQ ID NO:26, SEQ ID NO:28, SEQ ID NO:30, SEQ ID NO:32, SEQ ID NO:34, SEQ ID NO:36, SEQ ID NO:38, SEQ ID NO:40, SEQ ID NO:42, SEQ ID NO:44, SEQ ID NO:46, SEQ ID NO:48, SEQ ID NO:50, SEQ ID NO:52, SEQ ID NO:54, SEQ ID NO:56, SEQ ID NO:58, SEQ ID NO:60, SEQ ID NO:62, SEQ ID NO:64, SEQ ID NO:66, SEQ ID NO:68, SEQ ID NO:70, SEQ ID NO:72, SEQ ID NO:74, SEQ ID NO:76, SEQ ID NO:78, SEQ ID NO:80, SEQ ID NO:82, SEQ ID NO:84, SEQ ID NO:86, SEQ ID NO:88, SEQ ID NO:90, SEQ ID NO:92, SEQ ID NO:94, SEQ ID NO:96, SEQ ID NO:98, SEQ ID NO:100, SEQ ID NO:102, SEQ ID NO:104, SEQ ID NO:106, SEQ ID NO:108, SEQ ID NO:110, SEQ ID NO:112, SEQ ID NO:114, SEQ ID NO:116, SEQ ID NO:118, SEQ ID NO:120, SEQ ID NO:122, SEQ ID NO:124, SEQ ID NO:126, SEQ ID NO:128, SEQ ID NO:130, SEQ ID NO:132, SEQ ID NO:134, SEQ ID NO:136, SEQ ID NO:138, SEQ ID NO:140, SEQ ID NO:142, SEQ ID NO:144, SEQ ID NO:146, SEQ ID NO:148, or SEQ ID NO:150.

Such proteins have similar activities to those of Actinomadura sp. 21G792, particularly where there are conservative amino acid substitutions. A conservative amino acid substitution is defined as a change in the amino acid composition by way of changing one or more amino acids of a peptide, polypeptide or protein, or fragment thereof. The substitution is of amino acids with generally similar properties (e.g., acidic, basic, aromatic, size, positively or negatively charged, polarity, non-polarity) such that the substitutions do not substantially alter relevant peptide, polypeptide or protein characteristics (e.g., charge, isoelectric point, affinity, avidity, conformation, solubility) or activity. Typical conservative substitutions are selected within groups of amino acids, which groups include, but are not limited to:

(1) hydrophobic: methionine (M), alanine (A), valine (V), leucine (L), isoleucine (I);
(2) hydrophilic: cysteine (C), serine (S), threonine (T), asparagine (N), glutamine (Q);
(3) acidic: aspartic acid (D), glutamic acid (E);
(4) basic: histidine (H), lysine (K), arginine (R);
(5) aromatic: phenylalanine (F), tyrosine (Y) and tryptophan (W);
(6) residues that influence chain orientation: gly, pro.
Accordingly, the present invention also embraces apoproteins and polypeptides having similar amino acid compositions to the 21G792 apoprotein, wherein the amino acid sequences are substantially the same as SEQ ID NO:64 or SEQ ID NO:150, particularly where amino acid substitutions are conservative.

The proteins and polypeptides of the present invention can be isolated by any suitable method. For example, as stated above, when nucleotides encoding the apoprotein or pre-apoprotein are expressed in a host cell, the proteins can be expressed with an amino or carboxy terminus tag to facilitate isolation. Further, to isolate the polypeptides of the present invention from an actinomycete, especially where it is desired to isolate the apoprotein in a complex with an enediyne, one may follow a procedure similar to those described in the Examples below.

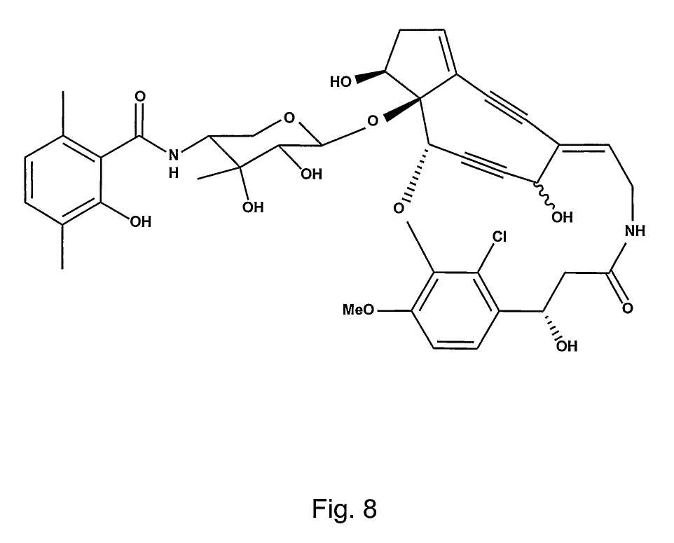

In an embodiment of the invention, the apoprotein is complexed with a chromophore. A preferred chromophore is that produced by Actinomadura sp. 21G792. The Actinomadura sp. 21G792 chromophore structure (FIG. 8) was deduced from the structure of a decomposed product that was generated by exposing the 21G792 chromoprotein to an organic solvent, and is related to the maduropeptin chromophore (see, Schroeder et al., 1994, J. Am. Chem. Soc. 116:9351; Zein, N. et al, 1995, Biochemistry 34, 11591-7).

The invention also provides methods for fermenting and cultivating Actinoinadura sp. 21G792. Cultivation of Actinomadura sp. 21G792 may be carried out in a wide variety of liquid culture media. Media which are useful for the production of the Actinomadura sp. 21G792 chromoprotein include an assimilable source of carbon, such as dextrin, sucrose, molasses, glycerol, etc.; an assimilable source of nitrogen, such as protein, protein hydrolysate, polypeptides, amino acids, corn steep liquor, etc.; and inorganic anions and cations, such as potassium, sodium, ammonium, calcium, sulfate, carbonate, phosphate, chloride, etc. Trace elements such as boron, molybdenum, copper, etc., are supplied as impurities of other constituents of the media.

The invention provides for changes to one or more orfs of the Actinomadura sp. 21G792 chromoprotein gene cluster, for example, by introduction of one or more random or targeted mutations, deletions, or insertions. In this manner, the chromophore, the apoprotein, or both may be modified in order to create a chromoprotein that exhibits, for example, decreased toxicity, increased potency, or increased stability. It is recognized that certain enediyne chromophores cleave DNA at sites specific to the chromophore. Further, various chromoproteins possess unique proteolytic activities towards histones. Accordingly, manipulation of the Actinomadura sp. 21G792 apoprotein and/or chromophore can also provide a chromoprotein with altered specificity. Alternatively, the apoprotein can be modified to serve as a carrier or delivery vehicle for an active molecule of choice. The invention also provides for a modified Actinomadura sp. 21G792 chromophore or apoprotein/chromophore complex that can be linked to another biological molecule. In one embodiment, the biological molecule provides for specific targeting of chromophore or chromoprotein. Such a biological molecule can be, for example, an antibody or other ligand for a cell surface molecule or receptor.

For example, a nucleic acid encoding an altered Actinomadura sp. 21G792 apoprotein can be inserted into an expression vector and into a host cell, the host cell cultured under conditions suitable for expression of the apoprotein, and the apoprotein recovered from the host cell or culture medium. Preferably, the host cell is capable of producing an enediyne chromophore or other molecule that can form a complex with the altered apoprotein. Examples of such cells include a variety of antibiotic producing organisms of the order Actinomycetales, particularly enediyne producing organisms such as Actinomadura and Streptomyces. Host cells further include common hosts such as E. coli and yeast. Of course, the altered apoprotein can be expressed in Actinomadura sp. 21G792. In one embodiment, the altered apoprotein will be over-expressed in the host cell. If any other endogenous apoprotein is present in the host cell, the altered apoprotein will be expressed at a higher level, the other apoprotein will be under-expressed, or the altered apoprotein will be expressed with a tag to facilitate such purification. In a preferred embodiment, the nucleic acid encoding the altered apoprotein is substituted for the endogenous apoprotein gene by homologous recombination. As such, the altered apoprotein can then be isolated in a complex with an enediyne or other molecule, e.g., an active agent, and then such a complex can be screened, e.g., against a cancer cell line, to determine bioactivity.

In yet another embodiment, a) the altered apoprotein is expressed in the host cell and is recovered without being complexed to an enediyne or other molecule, b) the altered apoprotein is then subjected to various enediyne or other molecules, c) an acceptable technique is used to determine whether the apoprotein forms a complex with the enediyne or other molecules, and optionally d) the complex is screened for bioactivity. In yet another embodiment, the altered apoprotein is expressed in the host cell and is recovered without being complexed to an enediyne or other molecule, the altered apoprotein is then subjected to various enediyne or other molecules, and the complex is screened for bioactivity.

In another example, nucleic acids encoding a modified chromophore biosynthetic pathway are expressed.

Functions of polypeptides expressed from the Actinomadura sp. 21G792 biosynthetic cluster may be deduced by comparing ORF sequences with known proteins and sequence motifs. (Table 3)

TABLE 3
Deduced functions for the Orfs of the 21G792 Chromoprotein Gene Cluster

| | | | Access. No.^{c}, | |
|---|---|---|---|---|
| ORF | Size^{a} | Similar Protein | (% id./% sim.) | Proposed Function |
| Orf9* | 462^{b} | ATP synthase beta subunit, AtpD, Nonomuraea sp. | AAU08241, n/a | primary metabolism |
| | | ATCC 39727 | | |
| Orf8* | 128 | ATP synthase epsilon chain, AtpC, Nonomuraea | AAU08242, 57/73 | primary metabolism |
| | | sp. ATCC 39727 | | |
| Orf7* | 151 | putative membrane protein, Streptomyces | BAC70590, 44/57 | primary metabolism |
| | | avermitilis MA-4680 | | |
| Orf6* | 532 | probable aminopeptidase, Thermobifida fusca YX | AAZ56436, 45/61 | primary metabolism |
| Orf5* | 211 | cobalamin adenosyltransferase, Thermobifida fusca | AAZ56437, 65/77 | primary metabolism |
| | | YX | | |
| Orf4* | 525 | GMC oxidoreductase, Deinococcus radiodurans R1 | AAF10542, 49/60 | primary metabolism |
| Orf3* | 68 | hypothetical protein, Oryza sativa | BAD81225, 41/52 | primary metabolism |
| Orf2* | 162 | acetyltransferases, Haemophilus somnus 2336 | ZP_00132424, 42/55 | primary metabolism |
| Orf1* | 498 | aldehyde dehydrogenase, Nocardioides sp. JS614 | ZP_00657819, 57/73 | primary metabolism |
| Orf1 | 331 | unknown, NcsE2, Streptomyces carzinostaticus | AAM78016, 62/69 | unknown |
| Orf2 | 300 | unknown, MadE3, Actinomadura madurae | AAQ17107, 100/100 | unknown |
| Orf3 | 678 | unknown, MadE4, Actinomadura madurae | AAQ17108, 99/99 | unknown |
| Orf4 | 330 | unknown, MadE5, Actinomadura madurae | AAQ17109, 100/100 | unknown |
| Orf5 | 1944 | Type I PKS, MadE, Actinomadura madurae | AAQ17110, 99/99 | Iterative type I PKS: KS, |
| | | | | AT, ACP, DH, KR, TD |
| Orf6 | 152 | putative thioesterase, MadE10, Actinomadura | AAQ17111, 100/100 | thioesterase |
| | | madurae | | |
| Orf7 | 183 | putative oxidoreductase, MadE6, Actinomadura | AAQ17112, 100/100 | oxidoreductase |
| | | madurae | | |
| Orf8 | 464 | putative P450 hydroxylase, MadE7, Actinomadura | AAQ17113, 99/99 | P450 hydroxylase |
| | | madurae | | |
| Orf9 | 251 | transcriptional regulator, NcsR5, Streptomyces | AAM78008, 52/65 | AraC family, |
| | | carzinostaticus | | transcriptional regulator |
| Orf10 | 336 | transcriptional regulator protein, KasT, | BAC53615, 49/63 | StrR-like transcriptional |
| | | Streptomyces kasugaensis | | regulator |
| Orf11 | 218 | putative regulatory protein, SgcR1, Streptomyces | AAL06694, 58/72 | unknown |
| | | globisporus | | |
| Orf12 | 552 | oxidoreductase, NcsE9, Streptomyces | AAM78005, 79/87 | oxidoreductase |
| | | carzinostaticus | | |
| Orf13 | 365 | unknown, SgcM, Streptomyces globisporus | AAL06686, 46/52 | unknown |
| Orf14 | 261 | unknown, NcsE11, Streptomyces carzinostaticus | AAM78004, 61/73 | unknown |
| Orf15 | 229 | O-methyltransferase, Frankia sp. EAN1pec | ZP_00573484, 49/67 | O-methyltransferase |
| Orf16 | 95 | NRPS PCP-domain, NRPS7-5, Streptomyces | BAB69396, 41/53 | aryl carrier protein |
| | | avermitilis MA-4680 | | |
| Orf17 | 1120 | type II NRPS A domain, SgcC1, Streptomyces | AAL06681, 41/49 | NRPS: C, A, PCP |
| | | globisporus | | |
| Orf18 | 537 | aminomutase, SgcC4, Streptomyces globisporus | AAL06680, 73/84 | aminomutase |
| Orf19 | 548 | putative halogenase, Frankia sp. Ccl3 | ZP_00548729, 62/75 | halogenase |
| Orf20 | 460 | type II NRPS C domain, SgcC5, Streptomyces | AAL06678, 46/59 | type II NRPS C domain |
| | | globisporus | | |
| Orf21 | 442 | squalene monooxygenase-like protein, SgcD2, | AAL06669, 50/56 | monooxygenase |
| | | Streptomyces globisporus | | |
| Orf22 | 525 | transmembrane efflux protein, SgcB, Streptomyces | AAF13999, 48/67 | transmembrane efflux |
| | | globisporus | | protein |
| Orf23 | 165 | hypothetical protein, Streptomyces avermitilis MA- | BAC71199, 33/44 | pre-apoprotein |
| | | 4680 | | |
| Orf24 | 461 | adenosylmethionine-8-amino-7-oxononanoate | BAD39928, 43/58 | aminotransferase |
| | | aminotransferase, Symbiobacterium thermophilum | | |
| Orf25 | 408 | P450 hydroxylase, Cyp28, Streptomyces avermitilis | BAC75180, 45/59 | P450 hydroxylase |
| | | MA-4680 | | |
| Orf26 | 381 | hypothetical protein, Streptomyces coelicolor A3(2) | CAC22728, 33/46 | unknown |
| Orf27 | 409 | putative cytochrome P450 oxidoreductase, | AAC25766, 45/60 | P450 oxidoreductase |
| | | Streptomyces lividans 1326 | | |
| Orf28 | 232 | conserved hypothetical protein, Bacillus clausii | AD63964, 51/71 | unknown |
| | | KSM-K16 | | |
| Orf29 | 466 | glycosyltransferase, SgcA6, Streptomyces | AAL06670, 43/57 | glycosyltransferase |
| | | globisporus | | |
| Orf30 | 285 | putative hydrolase, Streptomyces avermitilis MA- | BAC69810, 39/52 | epoxide hydrolase |
| | | 4680 | | |
| Orf31 | 548 | putative salicyl-AMP ligase, SdgA, Streptomyces | BAC78380, 54/64 | aryl acid-AMP ligase |
| | | sp. WA46 | | |
| Orf32 | 1746 | type I PKS, NcsB, Streptomyces carzinostaticus | AAM77986, 47/59 | iterative type I PKS: KS, |
| | | | | AT, DH, KR, ACP |
| Orf33 | 348 | O-methyltransferase, Trichodesmium erythraeum | ZP_00671263, 35/55 | C-methyltransferase |
| Orf34 | 393 | oxidoreductase, SgcL, Streptomyces globisporus | AAB13590, 67/78 | oxidoreductase |
| Orf35 | 148 | unknown, SgcT, Streptomyces globisporus | AAL06676, 61/76 | unknown |
| Orf36 | 401 | probable aminotransferase, SpnR, | AAG23279, 55/68 | aminotransferase |
| | | Saccharopolyspora spinosa | | |
| Orf37 | 447 | UDP-glucose dehydrogenase CalS8, | AAM70332, 52/63 | NDP-glucose |
| | | Micromonospora echinospora | | dehydrogenase |
| Orf38 | 328 | CalS9, Micromonospora echinospora | AAM70333, 61/71 | NDP-glucuronate |
| | | | | decarboxylase |
| Orf39 | 539 | chlorophenol-4-monooxygenase, SgcC, | AAL06674, 73/82 | aromatic ring hydroxylase |
| | | Streptomyces globisporus | | |
| Orf40 | 406 | putative C-3 methyl transferase, DvaC, | CAC48364, 58/74 | C-methyltransferase |
| | | Amycolatopsis balhimycina | | |
| Orf41 | 340 | alcohol dehydrogenase, Agrobacterium | AAK90613, 55/71 | alcohol dehydrogenase |
| | | tumefaciens str. C58 | | |
| Orf42 | 460 | squalene monooxygenase-like protein, SgcD2, | AAL06669, 60/72 | monooxygenase |
| | | Streptomyces globisporus | | |
| Orf43 | 347 | NDP-1-glucose synthase, med-ORF18, | BAC79029, 55/71 | dNDP-glucose synthase |
| | | Streptomyces sp. AM-7161 | | |
| Orf44 | 138 | putative lyase, Streptomyces coelicolor A3(2) | CAC37263, 47/61 | lyase |
| Orf45 | 557 | putative methylmalonyl-CoA decarboxylase alpha | BAC70414, 66/79 | carboxylyase/carboxyl |
| | | subunit, MmdA2, Streptomyces avermitilis MA-4680 | | transferase, lipid |
| | | | | metabolism |
| Orf46 | 230 | possible trancriptional regulator, Mycobacterium | CAD93534, 37/50 | TetR family, |
| | | bovix | | transcriptional regulator |
| Orf47 | 403 | retinal pigment epithelial membrane protein, | ZP_00577676, 31/40 | dioxygenase |
| | | Sphingopyxis alaskensis RB2256 | | |
| Orf48 | 444 | putative dioxygenase, SimC5, Streptomyces | AAK06796, 43/53 | dioxygenase |
| | | antibioticus | | |
| Orf49 | 277 | conserved hypothetical protein, Thermobifida fusca | AAZ55273, 51/64 | dNDP-sugar epimerase |
| | | YX | | |
| Orf50 | 213 | transcriptional regulatory protein, Bradyrhizobium | BAC49474, 45/60 | TetR family, |
| | | japonicum | | transcriptional regulator |
| Orf51 | 266 | putative membrane protein, Streptomyces | CAB61706, 52/66 | unknown |
| | | coelicolor A3(2) | | |
| Orf52 | 196 | putative TetR-family transcriptional regulator, | CAB71239, 30/47 | TetR family, |
| | | Streptomyces coelicolor A3(2) | | transcriptional regulator |
| Orf53 | 109 | transcriptional regulator, Mesorhizobium loti | BAB53793, 50/70 | ArsR family, |
| | | | | transcriptional regulator |
| Orf54 | 142 | conserved hypothetical protein, Ralstonia | CAD17332, 49/58 | unknown |
| | | solanacearum | | |
| Orf55 | 292 | LysR family regulatory protein, Frankia sp. | ZP_00571435, 43/54 | LysR family, |
| | | EAN1pec | | transcriptional regulator |
| Orf56 | 337 | class A beta-lactamase, Bla, Nocardia asteroides | AAG44836, 46/58 | unknown |
| Orf57 | 352 | hypothetical protein, Syntrophobacter fumaroxidans | ZP_00667098, 26/40 | unknown |
| Orf58 | 69 | none | — | unknown |
| Orf59 | 592 | RNA-directed DNA polymerase, Frankia sp. | ZP_00570947, 70/80 | unknown |
| | | EAN1pec | | |
| Orf60 | 59 | none | — | unknown |
| Orf61 | 25 | none | — | unknown |
| Orf62 | 167 | putative regulatory protein, Streptomyces coelicolor | CAC44216, 40/47 | regulator |
| | | A3(2) | | |
| Orf63 | 259 | conserved hypothetical protein, Streptomyces | CAB62713, 32/50 | unknown |
| | | coelicolor A3(2) | | |
| Orf64 | 144 | NUDIX hydrolase, Frankia sp. EAN1pec | ZP_00572338, 38/56 | DNA repair |
| Orf65 | 197^{b} | putative binding-protein-dependent integral | CAE50656, n/a | ABC transporter |
| | | membrane protein, Corynebactrium diphtheriae | | |

^{a}Numbers are in amino acids
^{b}Incomplete Orf
^{c}NCBI accession numbers of closest homologs are given
*Involved in primary metabolism

Consistent with those functions, a convergent biosynthetic pathway is provided for synthesis of the Actinomadura sp. 21G792 enediyne. Four primary components of the complex (enediyne core, madurosamine, 2-hydroxy-3,6-dimethyl benzoic acid, and 3-(2-chloro-3-hydroxy-4-methoxyphenyl)-3-hydroxy-propanoic acid) are produced separately and then assembled to form the final bioactive product.

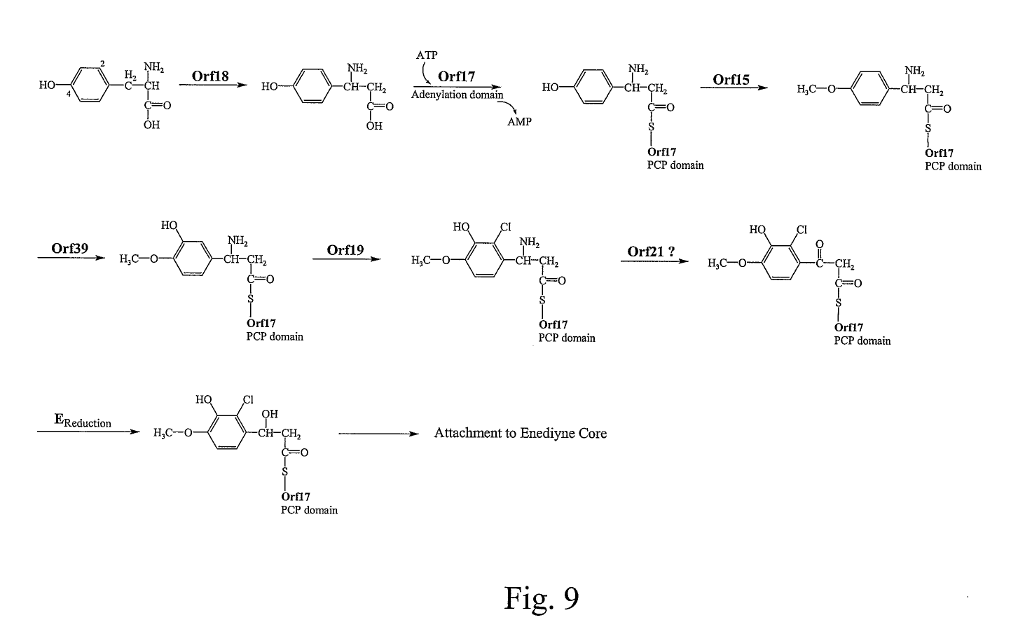

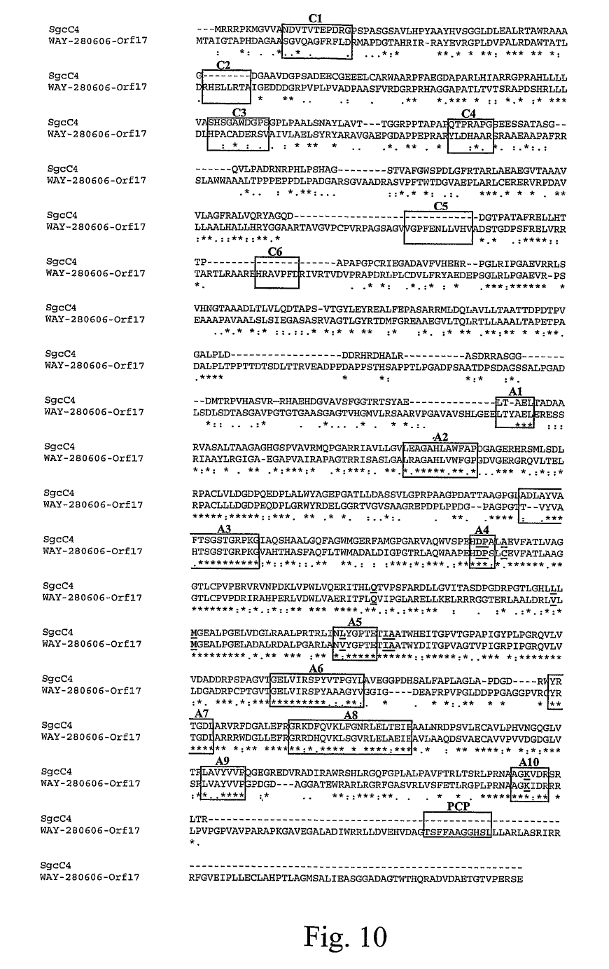

3-(2-chloro-3-hydroxy-4-methoxy-phenyl)-3-hydroxy-propanoic acid moiety biosynthesis. To produce the 3-(2-chloro-3-hydroxy-4-methoxy-phenyl)-3-hydroxy-propionic acid-derived portion of the enediyne (FIG. 9), tyrosine is first converted to β-tyrosine by the gene product of orf18. Orf18 shows high similarity to several histidine and phenylalanine ammonia lyases, but is most similar to SgcC4 of the C-1027 biosynthetic pathway (73% identity, 84% similarity), which catalyzes the conversion of α-tyrosine to β-tyrosine. (Liu et al., 2002, Science, 297, 1170-73, Van Lanen et al., 2005, J. Am. Chem. Soc., 127, 11594-5). Next, β-tyrosine is activated as an aminoacyl adenylate by the adenylation (A) domain of the orf17 gene product, and transferred to the sulfhydryl group of the phosphopantetheinyl prosthetic group on the adjacent peptidyl carrier protein (PCP), forming β-tyrosinyl-S-Orf17. Orf17 is similar to a wide array of nonribosomal peptide synthetases (NRPSs). Based on sequence analysis of the deduced amino acid sequence, Orf17 comprises three functional domains, a condensation (C) domain, an A domain and a PCP domain (FIG. 10). See, Konz and Marahiel, 1999, Chem. Biol., 6, R39-R47. The substrate specificity code of the A domain was extracted from the region between the A4 and A5 A domain structural motif, revealing the specificity code DPCQVMVIAK (Table 4). Table 4 also depicts the substrate and substrate specificity codes for SgcC1 from the C1027 biosynthetic cluster (Challis et al., 2000, Chem. Biol. 7, 211-24) and GrsA from the gramicidin biosynthetic cluster (Stachelhaus et al., 1999, Chem. Biol., 6, 493-505).

TABLE 4
Comparison of Adenylation Domain Substrate Specificity Codes

| | Amino Acid Position (GrsA numbering) | | | | | | | | | | |
|---|---|---|---|---|---|---|---|---|---|---|---|
| | 235 | 236 | 239 | 278 | 299 | 301 | 322 | 330 | 331 | 517 | Substrate |
| GrsA | D | A | W | T | I | A | A | I | C | K | Phe |
| Orf17 | D | P | C | Q | V | M | V | I | A | K | β-Tyr |
| SgcC1 | D | P | A | Q | L | M | L | I | A | K | β-Tyr |

Orf17 is most similar to SgcC1 from the C-1027 biosynthetic cluster (41% identity, 49% similarity). SgcC1 encodes a type II non-ribosomal peptide synthetase (NRPS) that is composed of a lone A domain. In vitro characterization of the enzyme has shown that it specifically activates β-tyrosine prior to loading it on SgcC2, a type II NRPS composed of a single PCP domain. (Van Lanen et al, 2005). Comparison of the substrate specificity codes of SgcCI and Orf17 reveals that the codes are remarkably similar (DPCQVMVIAK for Orf17 versus DPAQLMLIAK for SgcCI). This similarity is not surprising as both enzymes activate the same substrate. Interestingly, the stop codon of orf17 overlaps the start of orf18 by 3 bp, indicating that the expression of these two genes might be translationally coupled. Coordinating the expression of these genes is not unexpected, as expression of orf17 without the concurrent expression of orf18 to supply β-tyrosine, would result in the production of the orf17 gene product without a supply of its intended substrate.

Once loaded on the PCP of Orf17 via a thioester linkage, β-tyrosinyl-S-Orf17 is next methylated by Orf15 to give 3-amino-3-(4-methoxy-phenyl)-propanyl-S-Orf17. Orf15 shows strong similarity to many S-adenosylmethionine (SAM)-dependent O-methyltransferases and possesses three sequence motifs common to SAM-dependent methyltransferases (Motif I—VVDVGTFTG, SEQ ID NO:166; Motif 2—PAADLVFL, SEQ ID NO:167; Motif 3—LLRPGGLLVA, SEQ ID NO:168). Kagan and Clarke, (1994) Arc. Biochem. Biophys., 310, 417-427. As Actinomadura sp. 21G792 enediyne possesses a single O-methyl group, Orf15 is the enzyme most likely to catalyze this reaction. This enzyme-tethered intermediate is subsequently hydroxylated by Orf9 to yield 3-amino-3-(3-hydroxy-4-methoxy-phenyl)-propanyl-S-Orf17. BlastP analysis indicates that Orf39 is a hydroxylase similar to many hydroxylases responsible for the hydroxylation of phenolic substrates. It is strikingly similar to SgcC of the C-1027 biosynthetic cluster (73% identity, 82% similarity), which was shown, in vitro, to hydroxylate a chlorinated β-tyrosinyl-S-PCP intermediate. (Liu et al, 2002; Van Lanen et al., 2005). Following hydroxylation, the orf19 gene product chlorinates the C-2 position of the aromatic ring to yield 3-amino-3-(2-chloro-3-hydroxy-4-methoxy-phenyl)-propanyl-S-Orf17. Orf19 is homologous to several alkyl halidases involved in secondary metabolism, most notably SgcC3 from the C-1027 biosynthetic cluster (58% identity, 70% similarity), which has been shown to perform the chlorination of PCP bound β-tyrosine. (Liu et al, 2002; Van Lanen et al., 2005).

Since the β-tyrosine derivative incorporated into the Actinomadura sp. 21G792 enediyne bears a hydroxyl group in place of the amino group, one can envision the amino group of the 3-amino-3-(2-chloro-3-hydroxy-4-methoxy-phenyl)-propanyl-S-Orf17 intermediate being replaced by Orf21 via oxidative deamination. BlastP analysis reveals that Orf21 shows similarity to several putative FAD and NADPH-dependant monooxygenases/hydroxylases and domain analysis shows that it contains an FAD binding domain common to many monooxygenases. This domain is common to amino acid oxidases where oxidative deamination is well documented, thus Orf21 is a likely candidate to perform this transformation. It is important to note however, that there are several other candidates that could potentially catalyze this reaction including Orf42, which is also similar to FAD and NADPH-dependant monooxygenases/hydroxylases. Additionally, two Orfs (Orf25 and Orf27), which are similar to P450 hydroxylases, are present in the biosynthetic cluster and as P450 hydroxylases have also been implicated in oxidative deamination reactions, one of these enzymes might also catalyze this step. (Li et al., 2000, J. Bacteriol. 182, 4087-95) Following oxidative deamination, reduction of the ketone likely introduced by Orf21 or one of the other candidate enzymes, is likely to occur. The most obvious enzyme capable of catalyzing such a reaction would be a ketoreductase, similar to those employed in polyketide biosynthesis. Examination of the Actinomadura sp. 21G792 enediyne biosynthetic cluster did not identify any enzymes showing similarity to ketoreductase-like enzymes. There are several enzymes in the cluster that have unknown functions that might catalyze the required reduction, or the enzyme responsible for catalyzing the oxidative deamination might also catalyze the reduction reaction. Alternatively, an enzyme encoded outside of the current biosynthetic pathway could catalyze the expected reduction. Following ketoreduction the tyrosine derivative 3-(2-chloro-3-hydroxy-4-methoxy-phenyl)-3-hydroxy-propanyl-S-Orf17, is ready to be incorporated into the Actinomadura sp. 21G792 enediyne complex. The incorporation of this component of the Actinomadura sp. 21G792 enediyne into the final product is discussed below.

This synthetic pathway is not considered limiting but merely illustrative. Using this as a model, one of ordinary skill in the art can design numerous other synthetic schemes to produce the 3-(2-chloro-3-hydroxy-4-methoxy-phenyl)-3-hydroxy-propanyl component of the Actinomadura sp. 21G792 chromophore or a derivative of this component.

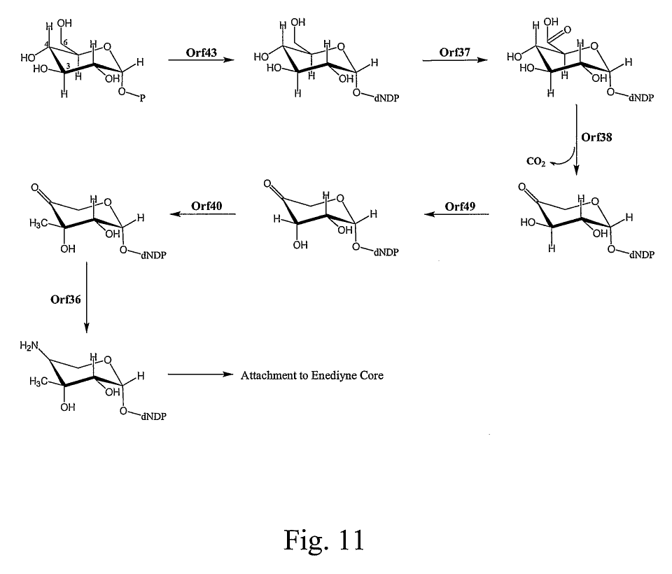

Madurosamine moiety biosynthesis. Analysis of the Actinomadura sp. 21G792 enediyne biosynthetic pathway identified five genes likely involved in madurosamine (4-amino-4-deoxy-3-C-methyl-β-ribopyranose) biosynthesis (FIG. 11). The first step in madurosamine (MDA) biosynthesis, as with all deoxysugars, is activation of D-glucose-1-phosphate (G-1-P) by a glucose-dNDP synthase. Trefzer et al., 1999, Nat. Prod. Rep. 16, 283-99. Orf43, which is homologous to several glucose-dNDP synthases, is responsible for activating G-1-P. Based on sequence homology of Orf43 to other proteins in the GenBank database, it likely catalyzes the formation of dTDP or dUDP-glucose.

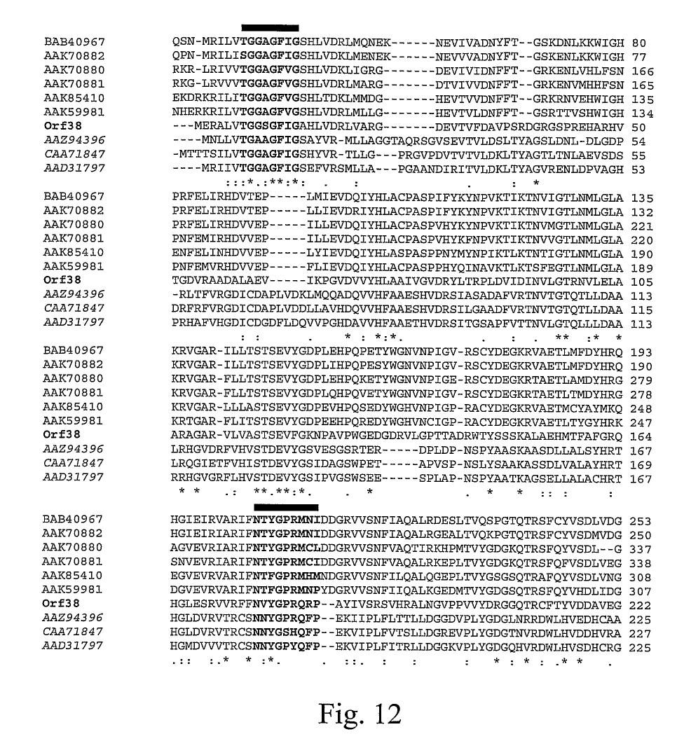

Next, Orf37, an enzyme highly homologous to dNDP-sugar dehydrogenases, oxidizes the primary alcohol to an acid, producing dNDP-D-glucuronate. Orf38, a probable dNDP-glucuronate decarboxylase, then converts dNDP-D-glucuronate to dNDP-xylose. A fragment amplified from orf38 was used as a probe to identify the first cosmid containing the Actinomadura sp. 21G792 enediyne biosynthetic cluster (See Examples) based on the prediction that biosynthesis of madurosamine might involve a dNDP-glucose-4,6-dehydratase including a 4,6-deoxyglucose intermediate. However, comparison of UDP-glucuronate decarboxylase and TDP-glucose-4,6-dehydratase amino acid sequences to that of Orf38 shows that the conserved amino acid motifs used by Decker et al. to design PCR primers used to amplify glucose-4,6-dehydratase genes, are also present in Orf8 and in the glucuronate decarboxylase sequences (FIG. 12). (Decker et al., 1994, FEMS Micro. Lett., 141, 195-201). Consequently it is not surprising that a glucuronate decarboxylase was amplified using these primers. Additionally, it should be noted that the stop codon of orf37 overlaps with the start codon of orf38, indicating that these orfs might be translationally coupled.

Following decarboxylation of dNDP-glucuronate, the C-3 hydroxyl of dNDP-D-xylose is epimerized by Orf49, producing dNDP-L-xylose. Orf49 is most similar to an uncharacterized protein from Thermobifida fusca (Accession no. AAZ55273.1) and its next most closely related homolog is ovmX (40% identity, 53% similarity), a putative NDP-sugar epimerase from Streptomyces antibioticus ATCC 11891 involved in the biosynthesis of oviedomycin. (Lombo et al., 2004, Chembiochem 5, 1181-7)

Following epimerization, the gene product of orf40 methylates the 3-carbon of dNDP-L-xylose. Orf40 shows significant similarity to a number of NDP-hexose C-methyltransferases and possesses three sequence motifs common to a wide variety of SAM dependent methyltransferases (Motif 1—IVEIGCNDG, SEQ ID NO:169; Motif 2—GPADVLYG, SEQ ID NO:170; Motif 3—LLKPDGIFVF, SEQ ID NO:171). (Kagan and Clarke, 1994, Arc. Biochem. Biophys., 310, 417-27). As a result, Orf40 is expected to perform this methylation. While another C-methylation is expected to occur in the biosynthesis of the 2-hydroxy-3,6-dimethyl-benzoic acid (HDBA) moiety of the Actinomadura sp. 21G792 enediyne, the C-methyltransferase expected to catalyze that methylation (Orf33), appears to form a small operon with the polyketide synthase responsible for generating the HDBA carbon skeleton, consequently Orf40 is not expected to participate in that transformation.

The methylated dNTP-sugar next undergoes C-4 transamination to form dNTP-madurosamine. This reaction is likely catalyzed by Orf36, which is highly homologous to SpnR (55% identity, 68% similarity) from the spinosyn biosynthetic cluster, which has been shown to carry out the C-4 transamination of a deoxysugar intermediate in the formation of D-forosamine. (Zhao et al., 2005, JACS, 127, 7692-3) The incorporation of the madurosamine component of Actinomadura sp. 21G792 enediyne into the final product will be discussed below.

This synthetic pathway is not considered limiting but merely illustrative. Using this as a model, one of ordinary skill in the art can design numerous other synthetic schemes to produce the MDA component of Actinomadura sp. 21G792 enediyne or a derivative of this component.

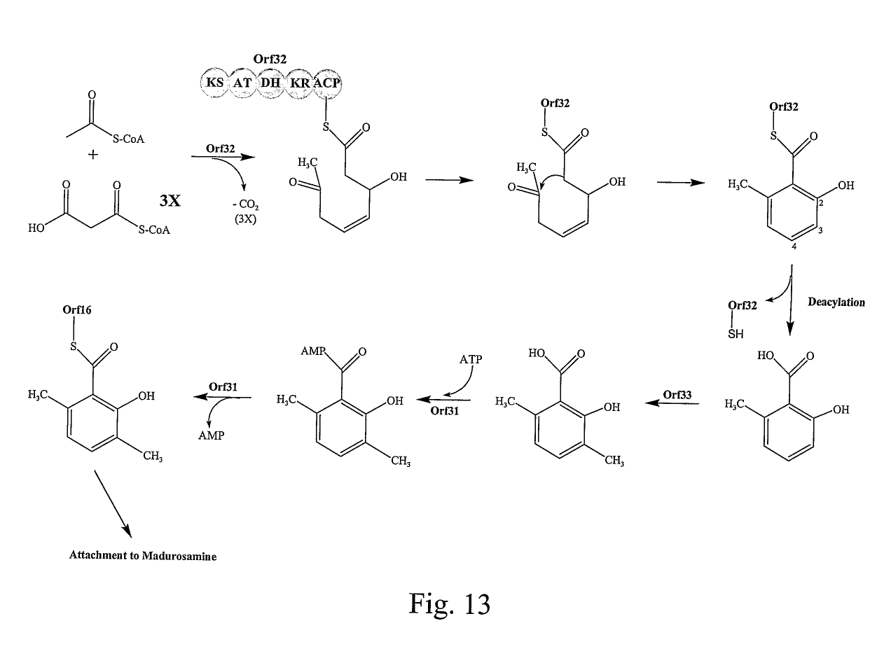

2-Hydroxy-3,6-dimethyl-benzoic acid moiety biosynthesis. The 2-hydroxy-3,6-dimethyl benzoic acid (HDBA) component of Actinomadura sp. 21G792 enediyne is most likely synthesized by two gene products, Orf32 an iterative type I polyketide synthase (PKS) and Orf33, a SAM-dependent C-methyltransferase (FIG. 13). Until recently, the bacterial paradigm for the biosynthesis of aromatic polyketides called for an iterative type II PKS. (Shen et. al., 2003, Curr. Opin. Chem. Biol. 7, 285-95) Examination of the Actinomadura sp. 21G792 enediyne biosynthetic cluster did not reveal the presence of any genes homologous to type II PKSs. Orf32, however, showed significant similarity to NcsB (47% identity, 59% similarity), an iterative type I PKS responsible for the production of the napthoic acid moiety of neocarzinostatin and to several 6-methylsalicylic acid synthases of fungal origin. (Liu et al., 2005, Chem. Biol., 293-302) Orf32 consists of 5 domains common to type I PKSs including a ketosynthase (KS), acyltransferase (AT), dehydratase (DH), ketoreductase (KR) and acyl carrier protein (ACP). It catalyzes the formation of a linear tetraketide from one acetyl-coenzyme A (coA) and 3 malonyl-coAs by iterative decarboxylative condensation followed by selective ketoreduction and dehydration at C-4 and ketoreduction at C-2. The nascent tetraketide intermediate then undergoes a nonenzymatic intramolecular aldol condensation to form the cyclized, 6-methylsalicylic (6MSA) acid intermediate.

The gene product of orf33 subsequently methylates the C-3 position of the 6MSA intermediate to form HDBA. Orf33 is similar to a wide variety of SAM-dependent methyltransferases including N-, C- and O-methyltransferases. Consistent with its classification, Orf33 possesses three sequence motifs common to a wide variety of SAM-dependent methyltransferases (Motif 1—VLDLGGGDG, SEQ ID NO:172; Motif 2—DGCDAILY, SEQ ID NO:173; Motif 3—ALPEGGVCVV, SEQ ID NO:174). (Kagan and Clarke, 1994) While the other methyltransferases present in the biosynthetic cluster might catalyze this reaction, Orf33 is immediately upstream of Orf32 and appears to be part of a small operon devoted to the production of HDBA and as a result, is the enzyme most likely to perform this reaction. Release of the cyclized polyketide from the PKS does not require a thioesterase, as is the case with most polyketides. Rather, it is released via a ketene pathway, analogous to that reported for 6-methylsalicylic acid biosynthesis. Spencer and Jordan, (1992) Biochem. J., 288, 839-846.

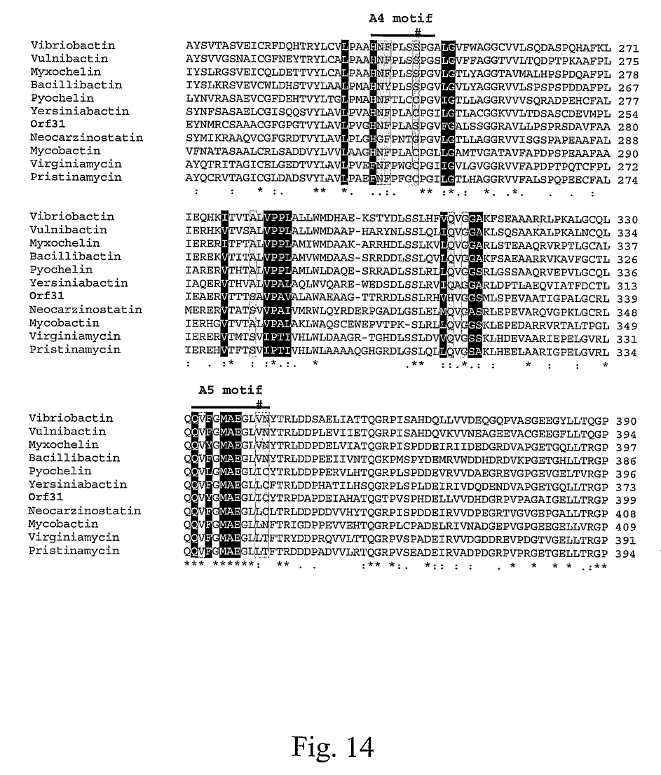

Following release from Orf32, HDBA is activated as an aryl adenylate by the gene product of orf31. Orf13 is similar to a number of aryl acid AMP-ligases. The best-studied examples of these types of enzymes come from investigations into siderophore biosynthesis. In the case of many siderophores, an aryl acid such as salicylate or 2′,3′-dihydroxybenzoate is adenylated as a first step in the assembly of the nonribosomal peptide core of the siderophore (see, Crosa and Walsh, 2002, Microbiol Mol. Biol. Rev., 66, 223-49 for a review). In addition to activating the aryl acid as an adenylate, these enzymes also transfer the aryl acids to the sulfhydryl group of the phosphopantetheinyl prosthetic group of a so-called aryl carrier protein (ArCP). Comparison of the crystal structure of the 2′,3′-dihydroxybenzoate-AMP ligase (DhbE) involved in the biosynthesis of the siderophore bacillibactin to that of other adenylating enzymes, including the NRPS GrsA adenylation domain and firefly luciferase revealed that aryl acid-activating domains contain a signature sequence not present in amino-acid activating domains. (May et al., 2002, PNAS 99, 12120-5). In DhbE, the so-called core A4 motif normally present in amino acid-activating domains (YxFDxS), is replaced by the sequence motif HNYPLSSPG. In amino acid-activating domains the invariant Asp residue stabilizes the α-amino group of the amino acid substrate, while in aryl acid-activating domains, the Asp residue is replaced with the conserved neutral Asn, which hydrogen bonds with the 2′-hydroxyl group of DHBA or salicylic acid. (May et al., 2002). As HDBA possesses a 2′-hydroxyl, one would expect Orf31 to possess the aryl acid-activating A4 motif. Examination of the Orf13 sequence revealed the motif HNFPLASPG (SEQ ID NO:175), which is consistent with enzymes activating aryl acids (FIG. 14).

As for amino acid-activating domains of NRPSs (Stachelhaus et al., 1999, Chem. Biol., 6, 493-505; Challis et al., 2000, Chem. Biol. 7, 211-24), a substrate specificity code for aryl acid-activating domains can be extracted from the region between the A4 and A5 core motifs. (May et al., 2002). Table 5 shows the comparison of the Orf31 substrate specificity code to substrate specificity codes of other aryl acid-activating domains involved in the biosynthesis of the following secondary metabolites: virginiamycin (VisB, accession number BAB83672), pristinamycin (SnbA, accession number CAA67140), mycobactin (MbtA, accession number CAB03759), yersiniabactin (YbtE, accession number AAC69591), pyochelin (PchD, accession number AAD55799), neocarzinostatin (NcsB2, accession number AAM77987), vibriobactin (VibE, accession number 007899), vulnibactin (Vva1301, accession number BAC97327), bacillibactin (DhbE, accession number AAC44632), and myxochelin (MxcE, accession number AF299336). Positions are numbered according to the GrsA phenylalanine-activating adenylation domain (Stachelhaus et al., 1999). Residues proposed to be involved in discrimination between the activation of 2′,3′-dihydroxybenzoic acid (DHBA) and salicylic acid are identified with an asterisk. Residues at each position matching that found in Orf31 are shaded in grey. HPA, 3-hydroxypicolinic acid.

Comparison of the Orf31 substrate specificity code to the codes of other aryl acid-activating enzymes and two enzymes that activate 3-hydroxypicolinic acid indicates that Orf31 activates either salicylic acid or HDBA. (Table 5).

TABLE 5
Comparison of aryl acid-activating domain substrate specificity codes

| | Amino Acid Position (GrsA numbering) | | | | | | | | | | |
|---|---|---|---|---|---|---|---|---|---|---|---|
| | 235 | 236 | 239* | 278 | 299 | 301 | 322 | 330* | 331 | 517 | Substrate |
| Virginiamycin | N | F | C | S | Q | G | V | L | T | K | HPA |
| Pristinamycin | N | F | C | S | Q | G | V | L | T | K | HPA |
| Mycobactin | N | F | C | A | Q | G | V | L | N | K | Salicylic acid |
| Yersiniabactin | N | F | C | A | Q | G | V | L | C | K | Salicylic acid |
| Pyochelin | N | F | C | A | Q | G | V | I | C | K | Salicylic acid |
| Neocarzinostatin | G | F | G | S | Q | G | V | L | C | K | Naphthoic acid |
| Orf31 | N | F | S | S | H | G | V | I | C | K | HDBA |
| Vibriobactin | N | F | S | A | Q | G | V | V | N | K | DHBA |
| Vulnibactin | N | F | S | A | Q | G | V | V | N | K | DHBA |
| Bacillibactin | N | Y | S | A | Q | G | V | V | N | K | DHBA |
| Myxochelin | N | F | S | A | Q | G | V | V | N | K | DHBA |

After activation of salicylic acid or HDBA, Orf31 catalyzes the transfer of the activated aryl acid to the sulfhydryl group of the phosphopantetheinyl prosthetic group attached to the ArCP, encoded by orf16. Orf16 is a small protein (95 aa), which is similar to many PCP and ArCP involved in secondary metabolism (˜30-40% identical) and it possesses the characteristic 4′-phosphopantheine attachment motif, including the invariant serine residue (GTFFQLRGQSI; SEQ ID NO:176). After attachment to the ArCP, the salicylate derivative is ready for incorporation into the Actinomadura sp. 21G792 enediyne complex, as discussed below.

This synthetic pathway is not considered limiting but merely illustrative. Using this as a model, one of ordinary skill in the art can design numerous other synthetic schemes to produce the 2-hydroxy-3,6-dimethylbenzoic acid component of Actinomadura sp. 21G792 chromophore or a derivative of this component.

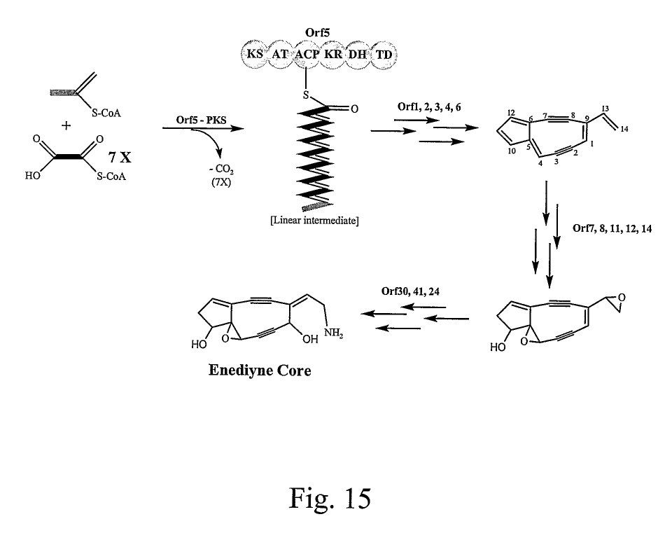

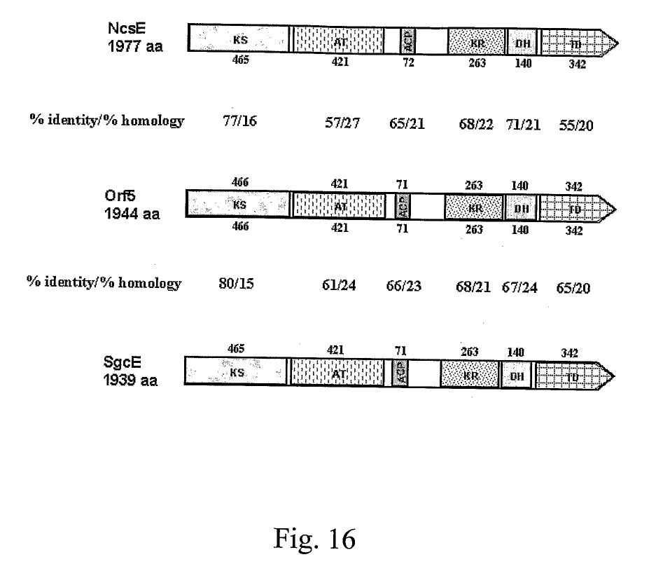

Enediyne core biosynthesis. At least fourteen genes were identified within the Actinomadura sp. 21G792 enediyne biosynthetic cluster whose deduced functions would support their roles in the Actinomadura sp. 21G792 enediyne core biosynthesis as outlined in FIG. 15. Orf5 encodes an iterative type I PKS that shows end-to-end sequence homology to the enediyne PKSs involved in the biosynthesis of neocarzinostatin (NcsE), C-1027 (SgcE) and calicheamicin (CalE8). (Liu et al., 2005; Liu et al., 2002; Ahlert et al., 2002, Science, 297, 1173-76). Like previously identified enediyne PKSs, Orf5 is composed of 6 domains: a KS, AT, ACP, KR, DH, and a so-called “terminal domain” (TD) (FIG. 16). The TD shows homology to 4′-phosphopantetheinyl transferases. Consequently, the TD has been proposed to catalyze the autoactivation of the enediyne PKS by post-translationally modifying the ACP active site serine with 4′-phosphopantetheine. (Zazopolous et al., 2003, Nature Biotech., 21, 187-90). Orf5 is expected to produce the nascent linear polyunsaturated polyketide intermediate from one acetyl-coA and 7 malonyl-coAs in an iterative fashion. The linear intermediate is possibly released from Orf5 and/or cyclized by Orf6, which shows similarity to a group of thioesterase proteins found in all enediyne biosynthetic clusters. Id. This group of proteins is predicted to function as thioesterases based on their homology to 4-hydroxybenzoyl-coA thioesterase of Pseudomonas sp. strain CBS-3. Id.

The polyketide intermediate is further processed by several gene products (Orfs 1-4, 7, 8, 11, 12, 14) to furnish the enediyne core (FIG. 15). These gene products are highly conserved in enedyine biosynthetic clusters. In addition to Orf5 and 6, homologs of Orfs 1-4 are found in all enediyne biosynthetic pathways studied to date (Id.), while homologs of Orfs 7, 8, 11, 12 and 14 are common to the 9-membered enediyne C-1027 and neocarzinostatin biosynthetic clusters. (Liu et al., 2005; Liu et al., 2002). Orfs 1-4, 11 and 14 are not homologous to any proteins of known function while Orfs 7, 8 and 12 resemble various oxidoreductases. Interestingly, it is possible that the expression of most of these genes is co-regulated, as orfs2-8 appear to be translationally coupled (e.g. the stop codon of orf2 overlaps the start codon of orf, and the stop codon of orf3 overlaps the start codon or orf4, etc.) as are orf11 and orf12.

The enediyne core (FIG. 15) is further modified by a minimum of three gene products, Orf30, Orf41 and Orf24, which are likely involved in producing a terminal amide from the C13-C14 epoxide of the enediyne core. orf30 encodes a probable epoxide hydrolase, orf41 encodes an alcohol dehydrogenase and orf24 encodes an aminotransferase. The fully modified enediyne core moiety is subsequently adorned with the other chromophore components to produce the active metabolite.

This synthetic pathway is not considered limiting but merely illustrative. Using this as a model, one of ordinary skill in the art can design numerous other synthetic schemes to produce the endiyne core of the Actinomadura sp. 21G792 chromophore or a derivative of this component.

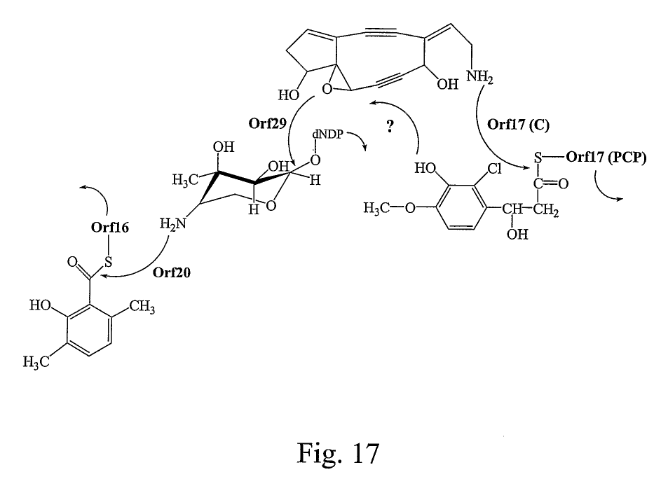

Assembly of the Actinomadura sp. 21G792 chromophore (FIG. 17). The biosynthesis of Actinomadura sp. 21G792 enediyne follows the current paradigm for enediyne biosynthesis, which calls for a convergent strategy for the assembly of the individual components of the molecular complex. (Liu et al., 2005; Liu et al., 2002; Ahlert et al., 2002). Following production of each component, they are systematically attached to the enediyne core to eventually furnish the final molecule as outlined in FIG. 17. The attachment of the enediyne core to the 3-(2-chloro-3-hydroxy-4-methoxy-phenyl)-3-hydroxy-propanyl-moiety is likely catalyzed by the condensation domain of Orf17. The catalysis of this reaction by Orf17 is consistent with the general peptide bond-forming activity normally attributed to the condensation domains of NRPSs. The mechanism used to attach the aromatic ring of the 3-(2-chloro-3-hydroxy-4-methoxy-phenyl)-3-hydroxy-propanyl-moiety to the enediyne core via ether bond formation is not known, however, it may occur concurrently with the opening of the C5-C6 epoxide and/or involve one or more of the P450 or monooxygenase encoding orfs contained within the Actinomadura sp. 21G792 enediyne biosynthetic cluster. The madurosamine moiety is coupled to the enediyne core via an O-glycosidic linkage. The gene product of orf29, which shows strong sequence similarity to a wide variety of glycosyltransferases involved in natural product biosynthesis, catalyzes this transfer. Orf29 is most similar to SgcA6 from the C-1027 biosynthetic pathway (43% identity, 57% similarity), which is proposed to catalyze the glycosylation of the C-1027 enediyne core. (Liu et al., 2002). Finally, Orf20, a type I NRPS condensation domain, transfers the HDBA-moiety from the phosphopatetheine arm of Orf16 to the amino group of madurosamine, in a reaction analogous to peptide bond formation in nonribosomal peptide biosynthesis.

Using this as a model, one of ordinary skill in the art can design numerous other synthetic schemes to produce the Actinomadura sp. 21G792 chromophore or a derivative of the chromophore.

The invention provides novel biosynthetic pathways comprising biosynthetic components of the Actimomadura sp. 21G792 chromophore, wherein one or more components has been mutated, or substituted or supplemented with a component from a biosynthetic pathway of a different enediyne chromophore, such that a variant of the Actinomadura sp. 21G792 chromophore is produced. Using standard molecular genetic techniques, individual orfs or combinations of orfs, as provided above, can be manipulated to produce novel bioactive analogs of the Actinomadura sp. 21G792 chromophore and/or chromoprotein. In one preferred embodiment, a novel chromophore is coexpressed with the Actinomadura sp. 21G792 apoprotein. In another embodiment, the Actinomadura sp. 21G792 chromophore is coexpressed with a variant of the Actinomadura sp. 21G792 apoprotein. In yet another embodiment, a novel chromophore is coexpressed with a variant of the Actinomadura sp. 21G792 apoprotein.

In an embodiment of the invention, inactivation of orf15 in Actinomadura sp. 21G792 produces an analog lacking the O-methyl that is usually found on the β-tyrosinyl moiety of the molecule. (See, e.g., FIG. 10) This change leaves a hydroxyl group in place of an O-methyl (see R¹ below). One reason for providing the hydroxyl group substitution would be to use it as a chemical handle for the further chemical derivitization of the analog by standard synthetic chemistry techniques. Similarly, inactivation of the halogenase encoded by orf19 prevents chlorination of PCP bound α-tyrosine, with the result that Cl is absent from the Actinomadura sp. 21G79 analog (see R² below). The R³ group indicated below is normally CH₃ and can be changed to H by inactivation the product of orf40 which methylates the 3-carbon of dNDP-L-xylose.

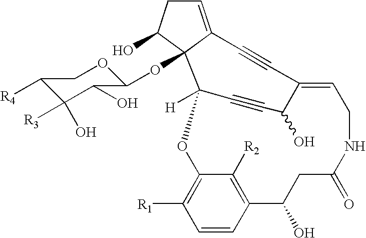

The R⁴ group of the Actinomadura sp. 21G792 chromophore is

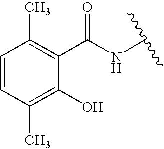

(designated R⁵), where R⁵ is linked to the sugar moiety at the amide nitrogen. Inactivation of orf32, causing production of an enediyne analog lacking the HDBA moiety (see, e.g., FIGS. 13, 17), or inactivation of orf20 results in substitution of R⁵ by NH₂. Further, the R⁴ moiety may be modified. For example,

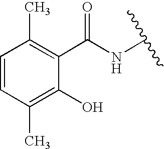

(designated R⁶) is obtained by inactivating orf33.

In another embodiment, orf32 is inactivated as above, and the mutant is used to produce a library of Actinomadura sp. 21G792 enediyne analogs where the HDBA moiety is replaced by other aryl acids. The aryl acids are introduced by feeding the orf32 mutant a variety of native aryl acids, N-acetyl cysteamine-linked aryl acids, or aryl acids linked to other thioester carriers such as methyl thioglycolate in the fermentation broth. (See, e.g., Jacobsen et al. (1997) Science 277, 367-9). Each of the orfs involved in the addition of a component to the Actinomadura sp. 21G792 molecular complex can be mutated singly and in combination with other orfs to produce a large library of Actinomadura sp. 21G792 enediyne analogs for biological testing.

Thus, the invention provides compounds having the formula:

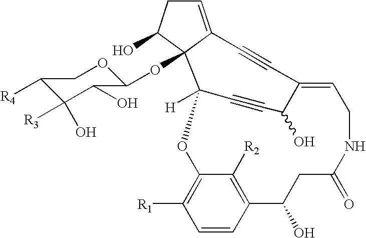

wherein R¹ is OH or OCH₃; R² is Cl or H; R³ is CH₃ or H; and R⁴ is selected from NH₂, R⁵, and R⁶. Further, by culturing an orf32 mutant in fermentation broth supplemented with particular native aryl acids, N-acetyl cysteamine-linked aryl acids, or aryl acids linked to other thioester carriers such as methyl thioglycolate, enediyne analogs can be produced wherein R⁴ is

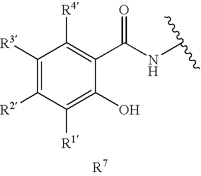

or

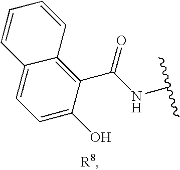

wherein R¹′ is H, CH₃, OH, OCH₃, C₁, C₃H₇, or NO₂; R²′ is H, CH₂, NH₂, OH, F, OCH₃, F, Cl, NO₂, OC₂H₅, or NC₂H₆; R³′ is H, CH₃, Cl, CH₃, NH₂, OH, F, COH, OCH₃, Cl, OC₂H₅, or NO₂; and R⁴′ is OH or OCH₃.

In other embodiments, one or more orfs from different secondary metabolic pathways can be introduced into Actinomadura sp. 21G792. Selected orfs can be introduced into the host chromosome by homologous recombination or by site specific integration mediated, for example, by a phage int/attP functionality (e.g. pSET152 or a similar vector). Alternatively selected orfs can be introduced on a self replicating vector. Once expressed, the gene products can proceed to modify the Actinomadura sp. 21 G792 chromophore. For example, sgcA, sgcA1, sgcA2, sgcA3, sgcA4, sgcA5 and sgcA6 from the C-1027 biosynthetic gene cluster could be introduced into an Actinoinadura sp. 21G792 strain in which one or more of the madurosamine biosynthetic orfs had been inactivated, in order to produce an Actinomadura sp. 21G792 enediyne analog comprising the C-1027 deoxy aminosugar, or a derivative thereof, in place of madurosamine.

The invention also provides for the introduction of genes from the chromoprotein biosynthetic cluster of Actinomadura sp. 21G792 into other secondary metabolite-producing microorganisms to modify the cognate secondary metabolite produced by that organism. For example, an analog of a different enediyne chromophore (e.g., the C-1027 chromophore) is produced by providing a host that expresses the biosynthetic pathway for that chromophore, and into which one or more of the components has been substituted or supplemented from the chromoprotein biosynthetic pathway of Actinomadura sp. 21G792.

In addition to making analogs of the Actinomadura sp. 21G792 chromoprotein, one can also increase fermentation titers by inactivating negative regulators as well as by increasing the expression level or gene copy number of positive regulators. The Actinomadura sp. 21G792 biosynthetic cluster contains at least eight orfs (orfs 9, 10, 46, 50, 52, 55, 62 and 63) identified as putative transcriptional regulators based on homology to sequences contained in the GenBank database. The function of these regulators can be tested in a systematic fashion to identify which regulator are positive regulators and which are negative regulators. Based on the findings, one could rationally alter one or more of these genes to increase fermentation titers of the Actinomadura sp. 21G792 chromoprotein.

Typically, organisms that produce toxic secondary metabolites possess one or more genes that confer self-resistance to the producing organism. The products of these genes usually confer resistance by chemically modifying, sequestering or transporting the toxic metabolite. In some cases, the target of the metabolite is innately insensitive to the metabolite, or the target is modified to confer insensitivity to the metabolite. The Actinomadura sp. 21G792 biosynthetic cluster contains at least two orfs whose gene products are likely involved in self-resistance. orf23, which encodes the apoprotein component of the Actinomadura sp. 21G792 complex, is presumably involved in sequestering the active chromophore, thereby shielding the DNA of the producing organism from cleavage by the chromophore. The gene product of orf22, encodes a protein similar to many transmembrane efflux proteins, and is most similar to SgcB from the C-1027 biosynthetic pathway, which has been proposed to act as an efflux pump for the C-1027 chromophore-apoprotein complex (Liu et al. (2005) Chem. Biol., 293-302). Using orf22 and orf23, one can potentially confer resistance to the Actinomadura sp. 21G792 chromoprotein. In one embodiment, these orfs can be introduced into a cell chosen to heterologously express the Actinomadura sp. 21G792 biosynthetic pathway, thereby allowing that cell to produce high levels of Actinomadura sp. 21G792 chromoprotein while being immune to its toxic effects. In another embodiment, these orfs can be introduced into donor cells chosen for biotransformation of Actinomadura sp. 21G792. Such cells would otherwise be killed by the extreme toxicity of Actinomadura sp. 21G792 before biotransformation could occur.

The entire Actinomadura sp. 21G792 biosynthetic cluster, or a selected portion, can be expressed in heterologous hosts such as bacteria. Examples of useful bacteria include, for example, members of the genera Streptomyces, Actinomadura, Nonomurea, Micromonospora, Escherichia, and Pseudomonas. (See, e.g., Pfeifer et al., 2001; Martinez et al., 2004) The biosynthetic cluster can also be heterologously expressed in a eukaryotic host such as yeast. In one embodiment, the Actinomadura sp. 21G792 biosynthetic cluster is advantageously expressed in an organism already modified for high level secondary metabolite production, thereby allowing for increased levels of Actinomadura sp. 21G792 chromoprotein production relative to that usually achieved using Actinomadura sp. 21G792. (See, e.g., Rodriguez et al., 2003, J. Ind. Microbiol. Biotechnol. 30, 480-8). In another embodiment, the Actinomadura sp. 21G792 biosynthetic cluster is advantageously expressed in an organism that is particularly amenable to genetic manipulation in order to expedite the generation of Actinomadura sp. 21G792 chromoprotein analogs (See, e.g., Bentley et al., 2002, Nature 417, 141-7; Binnie et al., 1997, Trends Biotechnol. 15, 315-20).

Various methods are known in the art that are useful for transferring recombinant DNAs encoding all or part of the Actinomadura sp. 21G792 chromoprotein biosynthetic pathway. Broad host-range plasmids are available that can be used to transfer and express such DNAs in a variety of hosts (e.g., pIJ101 for Streptomyces (Kieser et al., 1982, Mol. Gen. Genet. 185:223-8), pJRD215 for Actinomyces (Yeung et al., 1994, J. Bacteriol. 176:4173-6)). Methods for transferring such vectors include conjugation, electroporation and protoplast transformation. Shuttle vectors capable of replication in Escherichia coli and conjugal transfer from E. coli to gram-positive bacterial species such as Streptomyces spp. can also be used. (See, e.g., Mazodier et al., 1989, J. Bacteriol. 171:3583-5; Kieser et al., 2000, Practical Streptomyces genetics. A laboratory manual. John Innes Foundation, Norwich, United Kingdom).

It may be desired to prepare pharmaceutical compositions comprising a chromoprotein, wherein the chromoprotein comprises a complex of an apoprotein of the present invention and a chromophore, preferably the chromophore produced by Actinomadura sp. 21G792. Preferably, the polypeptide is attached to the chromophore via a non-covalent bond. Generally, preparing pharmaceutical compositions will entail preparing a pharmaceutical composition that is essentially free of pyrogens, as well as any other impurities that could be harmful to humans or animals. It may also be desirable to employ appropriate buffers to render the complex stable and allow for uptake by target cells.

Aqueous compositions of the present invention include an effective amount of the chromoprotein, further dispersed in a pharmaceutically acceptable carrier or aqueous medium. Such compositions also are referred to as inocula. The phrases “pharmaceutically or pharmacologically acceptable” refer to compositions that do not produce an adverse, allergic or other untoward reaction when administered to an animal, or a human, as appropriate.

As used herein, “pharmaceutically acceptable carrier” includes any and all solvents, dispersion media, coatings, antibacterial and antifungal agents, isotonic and absorption delaying agents and the like. The use of such media and agents for pharmaceutical active substances is well known in the art. Except insofar as any conventional media or agent is incompatible with the chromoproteins, its use in the therapeutic compositions is contemplated. Supplementary active ingredients, including antibacterial or anti-tumor agents, also may be incorporated into the compositions.

In an embodiment of the invention, a chromophore of the invention is taken up by a cell, for example, by pinocytosis. In another embodiment, the chromophore is modified so as to be targeted to a particular cell or cell type. In one such embodiment, a a chromoprotein may be delivered to target tissues in the form of polymers or conjugates employing monoclonal antibodies or other proteinaceous carriers as the targeting unit. Various polymer-based and antibody conjugate delivery systems are known and are currently being utilized in chemotherapeutic strategies involving the naturally-occurring C-1027 enediyne. In the present invention, the chromoproteins may, for example, be chemically-modified to form poly(styrene-co-maleic acid)-conjugated chromoproteins useful as therapeutics, particularly chemotherapeutics. (See, e.g., Maeda and Konno, 1997, in Neocarzinostatin: the Past, Present, and Future of an Anticancer Drug, H. Maeda, K. Edo, N. Ishida, Eds., Springer-Verlag, New York, pp. 227-267).

Polymeric micelles containing both hydrophobic and hydrophilic segments are new drug delivery systems recently developed to increase therapeutic indexes for chemotherapeutic agents (Yokoyama et al., 1990, Cancer Res. 50:1693-700; Kabanov et al., 1989, FEBS Lett. 258:343-5). Micelle size can be controlled so that the micelle particles are more permeable to blood vessels in tumor tissues than in normal tissues, owing to the enhanced permeability and retention (EPF) effect (Maeda, 2001, Adv Enzyme Regul. 41:189-207). This allows a favorable drug distribution in tumor tissues and hence the in vivo efficacy is expected to increase. The 21G792 chromoprotein can be non-covalently incorporated into specially designed micelles by mixing with a block copolymer solution. The metabolic stability of the resulting drug can be significant increased (Yokoyama et al., 1991, Cancer Res. 51:3229-36), which potentially is advantageous for delivering 21G792 chromoprotein in cancer chemotherapy.

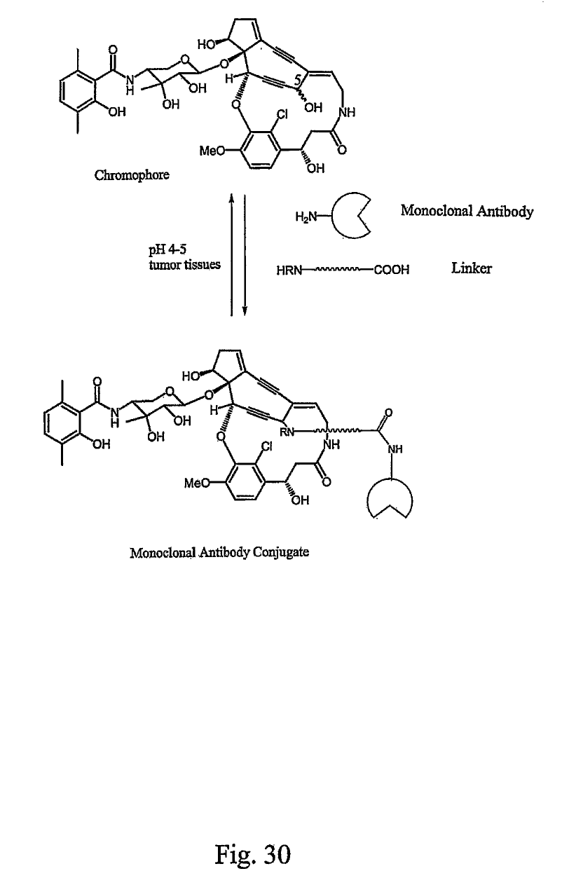

The chromoprotein (i.e., the apoprotein or chromophore) can be conjugated to a protein for delivery to a cell or a pathogen by the use of chemical linkers, or other related methods. The chromophore in the 21G792 chromoprotein has been reacted with sodium azide and secondary amines to give a series of derivatives. These derivatives contain an azide or secondary amino group at C-5 to replace the hydroxyl group in the natural chromophore. A linker with an amino group at one terminus and a carboxyl group at the other can be used to connect a monoclonal antibody and the chromophore to form a chromophore-antibody conjugate for targeted drug delivery. The amino group of the linker that is to replace the C-5 hydroxyl group is designed so that the conjugate can be hydrolyzed back to the chromophore under the more acidic condition in tumor tissues. An exemplary linkage is depicted in FIG. 30.

In addition, the chromoproteins may be conjugated with monoclonal antibodies to form monoclonal antibody (MAb)-chromoprotein conjugates. Antibodies with high affinity for antigens, preferably having specificity for antigenic determinants on the surface of malignant cells, are a natural choice as targeting moieties. Antibody-mediated specific delivery of the chromoproteins to tumor cells is expected to not only augment their anti-tumor efficacy, but also prevent nontargeted uptake by normal tissues, thus increasing their therapeutic indices. Examples of such antibody carriers that may be used in the present invention include monoclonal antibodies, chimeric antibodies, humanized antibodies, human antibodies, biologically active fragments thereof and their genetically or enzymatically engineered counterparts. Preferably, such antibodies are directed against cell surface antigens expressed on target cells and/or tissues in proliferative disorders such as cancer. The anti-CD33 monoclonal antibody is illustrative of a useful Mab for this approach and may effectuate the targeting of a chromoprotein to cancerous tissues in various contexts, including in patients afflicted with acute myeloid leukemia. (See, e.g., Sievers et al., 1999, Blood 93, 3678-84) Another example of a useful monoclonal antibody conjugate is described in PCT Publication No. WO 03/029623 in which, for example, an anti-CD22 monoclonal protein is conjugated to an enediyne for targeted delivery to B-cell lymphomas. As previously noted, several MAb-C-1027 conjugates are under evaluation as promising anticancer drugs. (Brukner, 2000, Curr. Opinion Oncologic, Endocrine & Met. Invest. Drugs 2, 344). Other proteinaceous carriers in addition to antibody carriers include hormones, growth factors, antibody mimics, and their genetically or enzymatically engineered counterparts, hereinafter referred to singularly or as a group as “carriers.” The essential property of a carrier is its ability to recognize and bind to an antigen or receptor associated with undesired cells and to be subsequently internalized. Examples of carriers that are applicable in the present invention are disclosed in U.S. Pat. No. 5,053,394, which is incorporated herein in its entirety. Preferred carriers for use in the present invention are antibodies and antibody mimics.

A number of non-immunoglobulin protein scaffolds have been used for generating antibody mimics that bind to antigenic epitopes with the specificity of an antibody (PCT publication No. WO 00/34784). For example, a “minibody” scaffold, which is related to the immunoglobulin fold, has been designed by deleting three beta strands from a heavy chain variable domain of a monoclonal antibody (Tramontano et al., 1994, J. Mol. Recognit. 7:9-24). This protein includes 61 residues and can be used to present two hypervariable loops. These two loops have been randomized and products selected for antigen binding, but thus far the framework appears to have somewhat limited utility due to solubility problems. Another framework used to display loops is tendamistat, a protein that specifically inhibits mammalian alpha-amylases and is a 74 residue, six-strand beta-sheet sandwich held together by two disulfide bonds, (McConnell and Hoess, 1995, J. Mol. Biol. 250:460-70). This scaffold includes three loops, but, to date, only two of these loops have been examined for randomization potential.

Other proteins have been tested as frameworks and have been used to display randomized residues on alpha helical surfaces (Nord et al., 1997, Nat. Biotechnol. 15, 772-7; Nord et al., 1995, Protein Eng. 8, 601-8), loops between alpha helices in alpha helix bundles (Ku and Schultz, 1995, Proc. Natl. Acad. Sci. USA 92, 6552-6), and loops constrained by disulfide bridges, such as those of the small protease inhibitors (Markland et al., 1996, Biochemistry 35, 8045-57; Markland et al., 1996, Biochemistry 35, 8058-67; Rottgen and Collins, 1995, Gene 164, 243-50; Wang et al., 1995, J. Biol. Chem. 270, 12250-6).

The targeting molecule and chromoprotein may be covalently associated by chemical cross-linking or through genetic fusion such as by application of recombinant DNA techniques. In the latter approach, the apoprotein may be fused at its C-terminus or N-terminus to the N-terminus or C-terminus of the cell targeting protein molecule. When the cell targeting molecule is an antibody, the C-terminus of the apoprotein is preferably fused to the N-terminus of the light and/or heavy chain of the antibody. For chemical cross-linking, some common protein-antibody linkers are succinate esters and other dicarboxylic acids, glutaraldehyde and other dialdehydes. Other such linkers are well known in the art.

Solutions of therapeutic compositions may be prepared in water suitably mixed with a surfactant (e.g., hydroxypropylcellulose). Dispersions also may be prepared in glycerol, liquid polyethylene glycols, mixtures thereof, and in oils. Under ordinary conditions of storage and use, these preparations contain a preservative to prevent the growth of microorganisms.

The therapeutic compositions of the present invention are advantageously administered in the form of injectable compositions either as liquid solutions or suspensions; solid forms suitable for solution in, or suspension in, liquid prior to injection may also be prepared. These preparations also may be emulsified. A typical composition for such purpose comprises a pharmaceutically acceptable carrier. For instance, the composition may contain 10 mg, 25 mg, 50 mg or up to about 100 mg of human serum albumin per milliliter of phosphate buffered saline. Other pharmaceutically acceptable carriers include aqueous solutions, non-toxic excipients, including salts, preservatives, buffers and the like.

Examples of non-aqueous solvents are propylene glycol, polyethylene glycol, vegetable oil and injectable organic esters such as ethyloleate. Aqueous carriers include water, alcoholic/aqueous solutions, saline solutions, parenteral vehicles such as sodium chloride, Ringer's dextrose, etc. Intravenous vehicles include fluid and nutrient replenishers. Preservatives include antimicrobial agents, anti-oxidants, chelating agents and inert gases. The pH and exact concentration of the various components of the pharmaceutical composition are adjusted according to well known parameters.

Additional formulations are suitable for oral administration. Oral formulations include such typical excipients as, for example, pharmaceutical grades of mannitol, lactose, starch, magnesium stearate, sodium saccharine, cellulose, magnesium carbonate and the like. The compositions take the form of solutions, suspensions, tablets, pills, capsules, sustained release formulations or powders. When the route is topical, the form may be a cream, ointment, salve or spray.

The therapeutic compositions of the present invention may include classic pharmaceutical preparations. Administration of therapeutic compositions according to the present invention will be via any common route so long as the target tissue is available via that route. This includes oral, nasal, buccal, rectal, vaginal or topical administration. Topical administration would be particularly advantageous for treatment of skin cancers, to prevent chemotherapy-induced alopecia or other dermal hyperproliferative disorder. Alternatively, administration will be by orthotopic, intradermal, subcutaneous, intramuscular, intraperitoneal or intravenous injection. Such compositions would normally be administered as pharmaceutically acceptable compositions that include physiologically acceptable carriers, buffers or other excipients. For treatment of conditions of the lungs, the preferred route is aerosol delivery to the lung. Volume of the aerosol is between about 0.01 ml and 0.5 ml. Similarly, a preferred method for treatment of colon-associated disease would be via enema. Volume of the enema is between about 1 ml and 100 ml.

An effective amount of the therapeutic composition is determined based on the intended goal. The term “unit dose” or “dosage” refers to physically discrete units suitable for use in a subject, each unit containing a predetermined-quantity of the therapeutic composition calculated to produce the desired responses, discussed above, in association with its administration, i.e., the appropriate route and treatment regimen. The quantity to be administered, both according to number of treatments and unit dose, depends on the protection desired.

Precise amounts of the therapeutic composition also depend on the judgment of the practitioner and are peculiar to each individual. Factors affecting dose include physical and clinical state of the patient, the route of administration, the intended goal of treatment (alleviation of symptoms versus cure) and the potency, stability and toxicity of the particular therapeutic substance.

### EXAMPLES

It is to be understood and expected that variations in the principles of the invention herein disclosed may be made by one skilled in the art and it is intended that such modifications are to be included within the scope of the present invention.

Examples of the invention which follow are set forth to further illustrate the invention and should not be construed to limit the invention in any way.

### Isolation and Characterization of the Chromoprotein and Apoprotein

### Example 1

### Isolation and Purification of the Actinomadura sp. 21G792 Chromoprotein

Actinomadura sp. 21G792 was preserved as frozen whole cells (frozen vegetative mycelia, FVM) prepared from cells grown for 72 hours in ATCC medium 172 (Dextrose 1%, Soluble Starch 2%, Yeast Extract 0.5%, and N-Z Amine Type A 0.5%, CaCO₃ 0.1% pH 7.3). Glycerol was added to 20% and the cells were frozen at −150° C.

A seed medium having a pH of 6.9 was prepared containing: 1.0% dextrose; 2.0% soluble starch; 0.5% yeast extract; 0.5% N-Z Amine Type A (Sheffield); and 0.1% CaCO₃. In a 25 mm×150 mm glass culture tube, 7 ml of the seed medium and two glass beads were inoculated with cells of Actinomadura sp. 21G792 cultured on ATCC agar medium #172 (ATCC Media Handbook, 1^{st} edition, 1984). Sufficient inoculum from the agar culture was used to provide a turbid seed after 72 hours of growth. The primary seed tubes were incubated at 28° C., 250 rpm using a gyro-rotary shaker with a 2 inch throw, for 72 hours. The primary seed (˜14% inoculum) was then used to inoculate a 250 ml Erlenmeyer flask containing 50 ml of medium #172. These secondary seed flasks were incubated at 28° C., 250 rpm using a gyro-rotary shaker (2″ stroke), for 48 hours.

A fermentation production medium having a pH of 6.9 was prepared containing: 2.0% sucrose; 0.5% molasses; 0.5% CaCO₃; 0.2% peptone; 0.002% magnesium sulfate-7H₂O; 0.001% ferrous sulfate-7H₂O; 0.05% sodium bromide; and 0.2% sodium acetate. Sixty 250 ml Erlenmeyer flasks were each prepared with 50 ml of the fermentation production medium and inoculated with 2 ml (4.0%) of the secondary seed fermentation and incubated at 28° C. at 250 rpm using a gyro-rotary shaker (2″ stroke). The fermentation as described was then allowed to proceed for approximately 72 to 96 hours and harvested for further processing.

The combined whole broth (60×50 ml) was centrifuged at 3800 rpm for 30 minutes. The supernatant was then lyophilized and the residual powder was suspended in a small volume (e.g., 300 ml) of H₂O. Upon centrifugation, the brownish solution was then loaded onto a glass column containing 6 L of Sephadex G75 in H₂O at 4° C. in the dark. Fractions of 40 ml each were collected and tested in a biochemical induction assay (BIA). The most potent fractions were then combined (15 fractions, 600 ml total) and lyophilized. The grayish powder was then dissolved in H₂O (4 ml) and analyzed by HPLC to contain two major peaks, one corresponding to the apoprotein and the other corresponding to the chromoprotein.

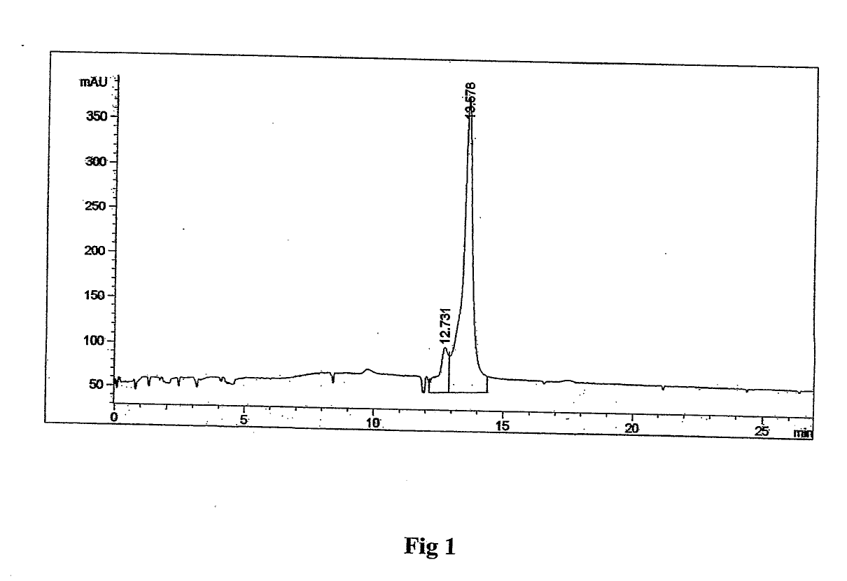

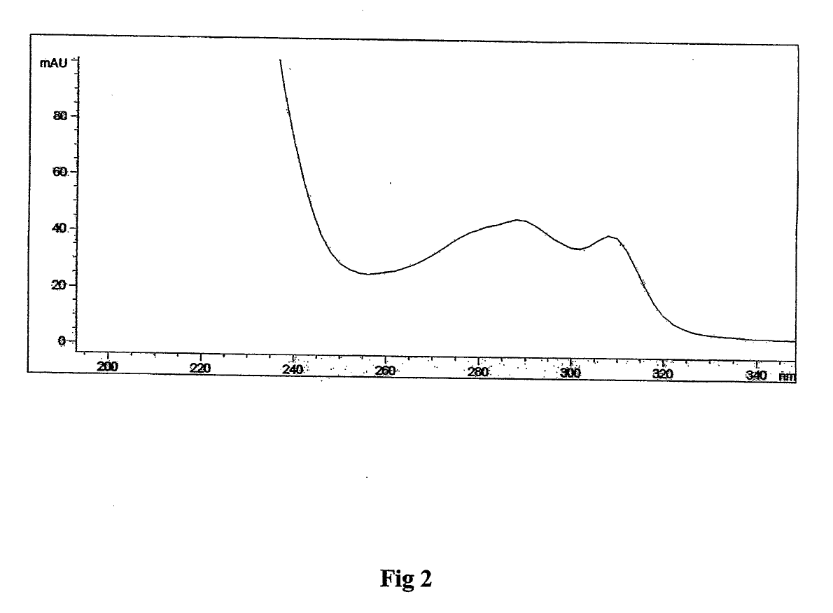

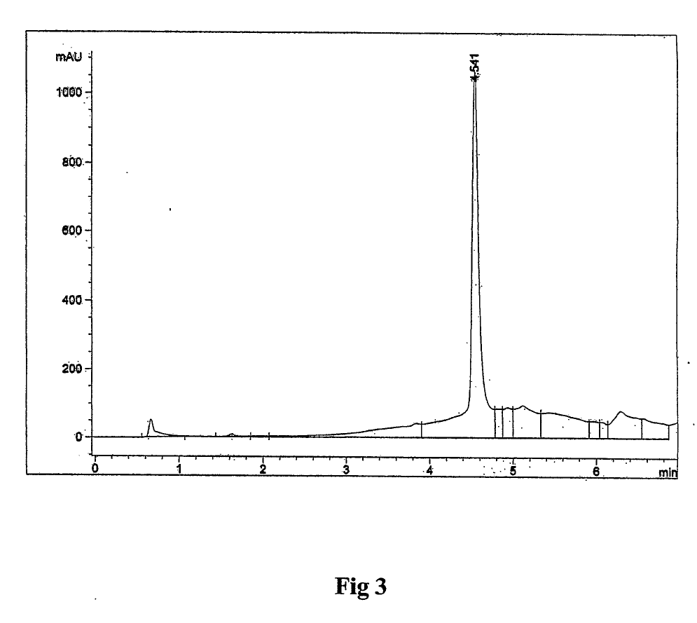

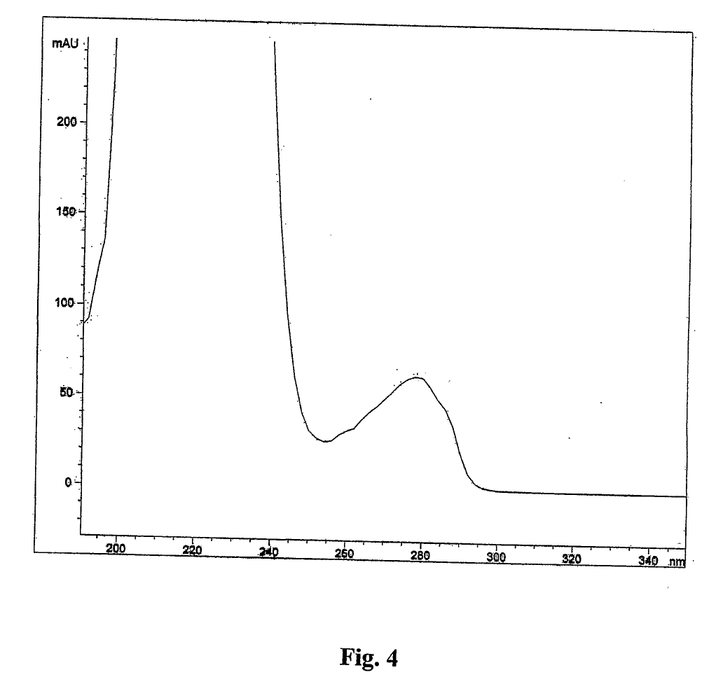

The above solution was subjected to preparative HPLC chromatography on a TosoHaas DEAE 5PW column (13 um particle size, 21.5 mm×15 cm in size) with a buffer system (0-0.5 M linear gradient NaCl with constant 0.05 M Tris-HCl in 30 min) at a flow rate of 4 ml/min. The respective peaks of apoprotein and chromoprotein were collected, desalted with Pierce Dialysis Cassette (7000 MWCO), and lyophilized. The resulting powders of apoprotein and chromoprotein were then repurified by the same preparative HPLC conditions, desalted, and lyophilized. The final products of chromoprotein (grayish powder, 10.5 mg) and apoprotein (white powder, 19.8 mg) were analyzed by analytical HPLC (FIGS. 1 and 3, respectively). The ultraviolet absorption (UV) spectra of the chromoprotein and apoprotein are shown in FIGS. 2 and 4.

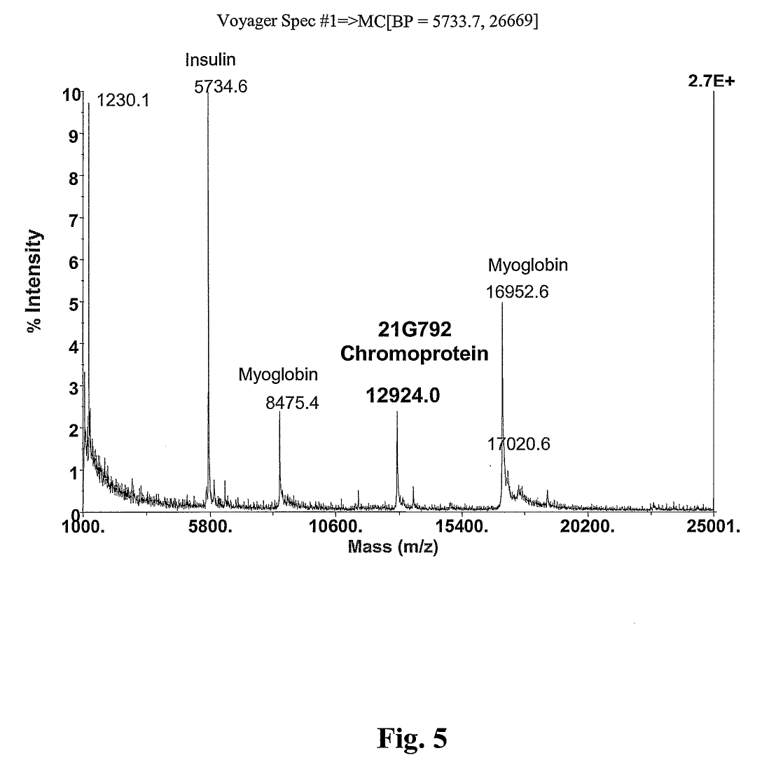

The molecular weight of the apoprotein was determined to be 12.92409 kDa by MALDI-MS. The MALDI spectrum is shown in FIG. 5.

### Example 2

### DNA Isolation and Sequencing of the Actinomadura sp. 21G792 Apoprotein

Genomic DNA was isolated from Actinomadura sp. 21G792 based on a modification to the procedure described in Hopwood et al. (1985), Genetic manipulations of Streptomyces. A Laboratory Manual. Norwich: John Innes Foundation. Approximately 1 ml of a frozen mycelia glycerol stock was inoculated into a 25 mm×150 mm seed tube containing 10 ml of MYM media (4 g/l maltose, 4 g/l yeast extract, 10 g/l malt extract, pH 7.0) and 2-6 mm glass beads. The culture was grown at 28° C. and 200 rpm for 5 days. The cells were then pelleted by centrifugation at 3000×g for 10 min. The supernatant was discarded and the pellet was suspended in 300 μl of T₅₀-E₂₀ (Tris 50 mM-EDTA-20 mM) containing 5 mg/ml lysozyme and 0.1 mg/ml RNase and incubated at 37° C. for 1 hr with gentle mixing every 15 min. 50 μl of 10% SDS was then added and the sample was thoroughly mixed. Next, 85 μl of 5 mM NaCl was added and the sample was again thoroughly mixed. The sample was then extracted with 400 μl phenol/chloroform/isoamyl alcohol (50/49/1). After vortexing the sample thoroughly, it was centrifuged at 10,000×g for 20 min at room temperature. Following centrifugation, the aqueous phase was removed and placed in a new microcentrifuge tube. An equal volume of room temperature isopropanol was added to the sample and thoroughly mixed by inversion. The sample was let stand at room temperature for 5 min. The sample was then centrifuged at 12,000×g for 30 min at 4° C. The isopropanol was carefully poured out of the tube and the DNA pellet rinsed with 1 ml of cold 70% ethanol. After being let stand in ice for 5 min, the 70% ethanol was poured out of the tube and the DNA was air dried for 10 minutes. The DNA was dissolved in 0.3 ml of sterile water. DNA integrity and concentration were estimated by agarose gel electrophoresis.

Escherichia coli; Plasmid and Small Scale Cosmid DNA preparations: Plasmid DNA and small-scale cosmid DNA preparations were performed using the Qiaprep Spin MiniPrep Kit (Qiagen Inc, Valencia, Calif., USA) according to the manufacturer's specifications. Cosmid: Cosmid DNA was isolated using the Qiagen Large Construct Kit (Qiagen Inc, Valencia, Calif., USA) according to the manufacturer's specifications.

An Actinomadura sp. 21G792 genomic library was constructed using the pWEB Cosmid Cloning Kit (Epicentre Technologies, Madison, Wis., USA) according to the manufacturer's specifications. The general library construction protocol was as follows. 10 μg of genomic DNA was randomly sheared into 30-45 kb fragments by passing the genomic DNA through a Hamilton HPLC/GC syringe. Following shearing, the fragmented DNA was end-repaired to produce blunt-ended fragments using the end-repair enzyme mix contained in the kit. The sheared and end-repaired DNA was then separated on a 1% low melting point agarose gel using linear T7 DNA (˜40 Kb) to serve as a molecular weight marker. Genomic DNA approximately equal in size to the T7 DNA was cut from the gel and the DNA was eluted from the agarose. The purified DNA was then ligated into the pWEB vector. Following ligation, the ligated insert DNA was packaged into lambda phage particles using the MaxPlax Lambda Packaging Extracts provided with the pWEB cosmid cloning kit. The phage extract was then titered to determine the colony-forming units per milliliter. Upon determining the titer of the phage extract, an appropriate amount of extract was used to infect E. coli EPI100 host cells and the infected cells were plated on Difco Luria agar plates containing 50 μg/ml of kanamycin to give a cell density of approximately 200 colonies per plate.

Library screening strategy and methodology; dNDP-glucose-4,6-dehydratase probe generation. Generally, the genes required to produce a particular antibiotic are clustered in the producing organism's genome. Further, there is precedence for clustering of an apoprotein gene with the genes encoding proteins involved in the biosynthetic pathway of the corresponding chromophore (Liu et al., 2002, Science 297:1170-3). The chromophore produced by Actinomadura sp. 21G792 contains the amino sugar 4-amino-4-deoxy-3-C-methyl-β-ribopyranose, which is attached to the enediyne core. Because a dNDP-D-glucose-4,6-dehydratase (DH) was expected to catalyze a step in the biosynthesis of this sugar, a DH probe was employed to isolate biosynthetic cluster.

To generate a DH probe, the polymerase chain reaction (PCR) was used to amplify a DH gene fragment from the genomic DNA of Actinomadura sp. 21G792. Primers for the expected ˜500 bp DH gene fragment (dehydra1: 5′-CSGGSGSSGCSGGSTTCATSGG (SEQ ID NO:152) and dehydra2: 5′-GGGWRCTGGYRSGGSCCGTAGTTG (SEQ ID NO:153)) were identical to those described by Decker et al., 1996, FEMS Microbiol. Lett. 141, 195-201. PCR was conducted using JumpStart REDTaq Ready Mix PCR Reaction Mix (Sigma-Aldrich Corp, St. Louis, Mo.) according to the manufacturer's specifications. The primers were used at a final concentration of 0.5 μM. PCR was performed on a Biometra T gradient thermocycler. The starting denaturing temperature was 96° C. for 4 min. The following 30 cycles were as follows: denaturing temperature 96° C. (45 sec), annealing temperature 66° C. (45 sec), extension temperature 72° C. (3 min). At the end, the final extension temperature was 72° C. for 10 min.

The ˜500 bp amplicon was cloned into pCR2.1 using the TOPO TA Cloning Kit (Invitrogen Corp, Carlsbad, Calif.) following the manufacturer's recommendations. A portion (2.5 μl) of the cloning reaction was used to transform E. coli TOP10 cells (Invitrogen Corp, Carlsbad, Calif.) which were subsequently plated on Difco Luria Agar containing 50 μg/ml kanamycin, 40 μg/ml X-gal and 0.2 mM IPTG to facilitate blue/white screening of recombinant clones. Twenty white colonies were picked and their plasmid DNA was isolated. Sequencing of these clones revealed that two different DH gene fragments had been cloned. Comparison of the deduced amino acid sequences revealed that one of the DH fragments (contained in plasmid p34598) was most similar to a DH involved in calicheamicin biosynthesis. As the calicheamicin structure contains 2 amino sugars, it was predicted that the DH fragment contained in p34598 might also be involved in amino sugar production, and thus was chosen as the probe for the chromoprotein gene cluster.

Colony hybridization: The Actinomadura sp. 21G792 genomic library was screened by colony hybridization using the p34598 DH fragment. Recombinant colony DNA was transferred to Nytran SuPerCharge nylon membrane discs (Schleicher & Schuell BioScience, Inc., Keene, N.H.) as described by Sambrook and Russell (2001), Molecular Cloning, A Laboratory Manual, Cold Spring Harbor Laboratory Press (3^{rd} ed.). The DH probe was prepared using PCR and primers dehydra1 and dehydra2 to amplify the insert of p34598. The amplified PCR product was separated by agarose gel electrophoresis and the 530 bp fragment was isolated from the agarose. This fragment was then labeled with [α-³²P]dCTP (3000 Ci/mmol Amersham Bioscience, Piscataway, N.J.) using the Megaprime DNA Labeling kit according to the manufacturer's specifications (Amersham Bioscience, Piscataway, N.J.). The nylon membrane on which the DNA samples were immobilized was washed in 6×SSC, then placed in a hybridization bottle with prewarmed (65° C.) prehybridization solution (6×SSC/5×Denhardt's reagent/0.5% (w/v) SDS and 100 μg/ml of denatured, sheared herring sperm DNA) and “pre-hybridized” for 2 h. The denatured probe was then added, and hybridization proceeded overnight at 65° C. The following day the membrane was washed once with prewarmed (65° C.) 2×SSC/0.1% SDS (Wash Solution 1) for 1 h and once with prewarmed (65° C.) 1×SSC/0.1% SDS (Wash Solution 2) for 1 h. The nylon membrane was then wrapped in Saran wrap and exposed to Kodak X-omat AR film for 4 h. The exposed films were developed using a Kodak X-omat 2000A processor. Twenty-two colonies appeared to hybridize to the probe. These colonies were picked and grown in Difco Luria Broth containing 50 μg/ml kanamycin. The cosmid DNA was purified from the cultures and cut with Not I. The restriction digests were separated by agarose gel electrophoresis and the DNA was transferred to a Nytran SuPerCharge nylon membrane as described by Sambrook and Russell (2001). This membrane was probed using the same conditions used for the colony hybridization, again using the p34598 insert as a probe. Nine cosmids positively hybridized to the probe. The cosmids and approximate sizes of the fragments that hybridized to the probe were: 21gB: 15-20 kb, 21gC: 15-20 kb, 21gD: 8-12 kb, 21gF: 15-20 kb, 21gG: 3-4 kb, 21gI: 1.2-2.5 kb, 21gK: 15-20 kb, 21gL: 2.5-3 kb, 21gV: 2-2.5 kb.

Apoprotein—specific oligonucleotide probe hybridization: Edman protein sequencing was used to determine the first 38 amino acid residues of the apoprotein, N-terminus DTVTVNYDDVGYPSDIAVTIDAPATAGVGDTATFEVSV (SEQ ID NO:154). To definitively identify which cosmids might contain the apoprotein gene sequence, a hybridization experiment was conducted using, as a probe, a degenerate oligonucleotide that was based on residues 4-12 of the 38 amino acid (aa) sequence of the apoprotein N-terminus. Specifically, the sequence of the oligonucleotide was 5′-ACSGTSAACTACGACGACGTSGGNTAC (SEQ ID NO:155).

The cosmids that hybridized to the DH probe were digested with Not I and transferred to a Nytran SuPerCharge nylon membrane. The oligonucleotide was end labeled with [γ-³²P]dATP (6000 Ci/mmol; Amersham Bioscience, Piscataway, N.J.) using the KinaseMax 5′ End-Labeling Kit according to the manufacturer's recommendations (Ambion Inc., Austin, Tex.). Unincorporated radioactive nucleotides were removed using the NucAway Spin Column Kit according to the manufacturer's directions (Ambion Inc., Austin, Tex.). The DNA-carrying nylon membrane was “pre-hybridized” for 3 h at 50° C. in a solution containing 6×SSC, 5×Denhardt's reagent, 0.05% sodium pyrophosphate, 0.5% SDS and 100 μg/ml sheared and denatured salmon sperm DNA. Following this step, the pre-hybridization solution was replaced with 7 ml pre-warmed (50° C.) hybridization solution containing 6×SSC, 0.5% sodium phosphate, 1×Denhardt's reagent and 100 μg/ml yeast tRNA. The labeled probe was added to this solution and the hybridization was incubated at 50° C. for 22 h. Next, the hybridization solution was discarded and the membrane was rinsed briefly with 20 ml of room temperature TMACL wash buffer (3 M TMACL, 50 mM Tris, 0.2% SDS). It was then washed with an additional 50 ml of pre-warmed (67° C.) TMACL wash buffer for 55 min at 67° C. For the final wash, the membrane was washed with 50 ml of pre-warmed (50° C.) Wash Solution 1 for 10 min at 50° C. The membrane was then wrapped in Saran wrap and exposed to Kodak X-omat AR film for 24 h.

Cosmids 21gD, 21gG and 21gK hybridized to the probe. An ˜4.5 kb signal was observed in the lanes containing 21gD and 21gK DNA, while an ˜5.2 kb signal was observed in the lane containing 21gG DNA. To confirm this hybridization result, PCR was conducted using 21gD cosmid DNA as the template and degenerate PCR primers designed to amplify a 98 bp fragment from the apoprotein. The PCR primers CP-FWD3 (5′-ACSGTSAAYTAYGAYGAYGT; SEQ ID NO:156) and CP-REV4 (5′-ACYTCRAASGTSGCSGTRTC; SEQ ID NO:157) were designed using the reverse translated DNA sequence deduced from the 36 aa sequence of the apoprotein. PCR was performed using JumpStart REDTaq Ready Mix PCR Reaction Mix (Sigma-Aldrich Corp, St. Louis, Mo.) according to the manufacturer's specifications. The primers were used at a final concentration of 2.0 μM. The PCR was performed on a Biometra Tgradient thermocycler. The starting denaturing temperature was 96° C. for 4 min. The following 5 cycles were as follows: denaturing temperature 96° C. (45 sec), annealing temperature 40° C. (45 sec), extension temperature 72° C. (2 min). The next 30 cycles were as follows: denaturing temperature 96° C. (30 sec), annealing temperature 55.7-72.0° C. (45 sec; 8 temperatures tested within range), extension temperature 72° C. (2 min). At the end, the final extension temperature was 72° C. for 10 min. Several bands were generated by these conditions; however, using annealing temperatures 55.7° C., 58.6° C. and 61.4° C., an intense band of approximately 100 bp was generated. The 100 bp amplicon was cloned into pCR2.1 using the TOPO TA Cloning Kit (Invitrogen Corp, Carlsbad, Calif.) following the manufacturer's recommendations. A portion (2.5 μL) of the cloning reaction was used to transform E. coli TOP10 cells (Invitrogen Corp, Carlsbad, Calif.) which were subsequently plated on Difco Luria Agar containing 50 μg/ml kanamycin, 40 μg/ml X-gal and 0.2 mM IPTG to facilitate blue/white screening of recombinant clones. Ten white colonies were picked and their plasmid DNA isolated. Sequencing of these clones revealed that 4 clones (p35546, p35547, p35550, p35554) contained DNA whose deduced amino acid sequence matched that of the 36 aa apoprotein fragment exactly, thus confirming that the gene encoding the apoprotein was contained in cosmid 21gD.

Elucidation of complete apoprotein DNA sequence in cosmid 21gD. To determine the full sequence of the gene encoding the apoprotein, sequencing primers were designed from the DNA sequence of the 98 bp PCR product amplified above. The following primers were used for the initial round of sequencing using cosmid 21gD as a template:

| | |
|---|---|
| ApoSeqCode1: | |
| 5′-GGCTACCCGTCGGACATCG; | (SEQ ID NO:158) |
| ApoSeqCode2: | |
| 5′-GGACATCGCCGTGACCATCG; | (SEQ ID NO:159) |
| ApoSeqComp1: | |
| 5′CCGGCGCGTCGATGGTCAC; | (SEQ ID NO:160) |
| ApoSeqComp2: | |
| 5′-CTCGAAGGTGGCGGTGTC. | (SEQ ID NO:161) |

The first round of sequencing generated 1440 bp of sequence. Using the CodonPreference program, a small 498 bp open reading frame (ORF) was identified. Comparison of the deduced amino acid sequence of this orf to the partial amino acid sequence of the Actinomadura sp. 21G792 apoprotein (determined by Edman protein sequencing) confirmed that the ORF did encode the apoprotein, as the two amino acid sequences were identical. Additionally, the molecular weight of the deduced amino acid sequence, 12926 Da, was in good agreement with the molecular weight of the apoprotein as determined by high resolution MALDI MS, 12924.09. Also, the DNA sequence of the apoprotein was confirmed further by extensive sequencing of both DNA strands using primers flanking the orf encoding the apoprotein (designated aseA).

The deduced amino acid sequence of the pre-apoprotein, which contains the leader peptide and the apoprotein, is provided in SEQ ID NO:64. The nucleotide sequence encoding the pre-apoprotein is provided in SEQ ID NO:63. The deduced amino acid sequence of the apoprotein is provided in SEQ ID NO:150. The nucleotide sequence encoding the apoprotein is provided in SEQ ID NO:149. Finally, a figure describing the DNA sequence of the pre-apoprotein, the corresponding amino acid sequence, the putative upstream ribosome binding site, and the splitting site between the leader peptide and apoprotein is provided in FIG. 6.

### Example 3

### DNA Isolation and Sequencing of the Remainder of the Actinomadura sp. 21G792 Chromoprotein Biosynthetic Cluster

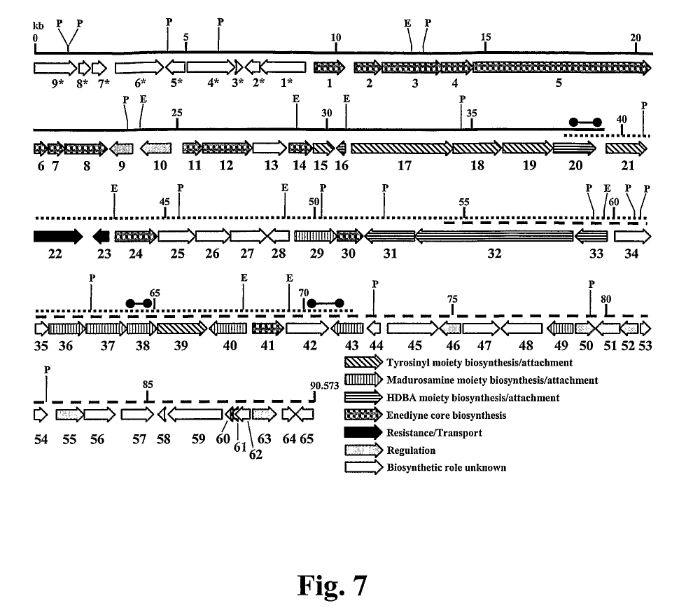

Identification of distal sequences of the Actinomadura sp. 21G792 apoprotein gene cluster. Sequences adjacent to the portion of the Actinomadura sp. 21G792 apoprotein gene cluster present in cosmid 21gD were identified as described below. Along with cosmid 21gD, these sequences are thought to constitute substantially the entire biosynthetic cluster of the Actinomadura sp. 21G792 chromoprotein—i.e. the genes responsible for assembling the chromoprotein. Locations of the open reading frames are identified in Table 1. Functions of the encoded proteins were deduced by comparison with GenBank sequence deposits (Table 3). The arrangement of the open reading frames is depicted in FIG. 7.

First, a probe was generated from cosmid 21gD by amplifying a 904 bp fragment from the end of the cosmid containing the partial type II peptide synthetase condensation domain (orf20; FIG. 7) using primers 21gDpr1FWD (5′-GCTCGTCGGGTTCTTCTAC; SEQ ID NO:162) and 21gDpr1REV (5′-GACTTCGCGATAGCTCTC; SEQ ID NO:163). PCR amplification was conducted using KOD polymerase (Novagen) with 5% DMSO according to the manufacturers recommendations. Primers were used at a concentration of 0.5 mM. Cosmid 21gD was used as template DNA. The cycling conditions were as follows: 1 cycle of 96° C. for 2 min, followed by 30 cycles of 96° C. for 1 min, 61.2° C. for 1 min, and 72° C. for 2 min, followed by 1 cycle of 72° C. for 10 min. The PCR reaction was examined by agarose gel electrophoresis and the 904 bp band was eluted from the agarose as previously described. The 904 bp amplicon was used to probe the Actinomadura sp. 21G792 genomic cosmid library as previously described for the 4,6-dehydratase probe. 38 colonies that hybridized to the probe were cultured (5 ml Difco Luria Broth containing 50 μg/ml kanamycin) and cosmid DNA was purified. The purified cosmids were end sequenced using sequencing primer sites contained in the pWEB vector. Analysis of the DNA sequences indicated that one cosmid (41417) overlapped with cosmid 21gD by 1184 bp. Cosmid 41417 was subsequently sequenced in its entirety, open reading frames were identified, and functions of the encoded proteins were deduced.

The portion of the biosynthetic cluster distal to the other end of cosmid 21gD was identified by screening the cosmids previously identified as having hybridized to the putative dNDP-D-glucose-4,6-dehydratase fragment cloned in p34598 (used to identify cosmid 21gD). These cosmids were screened using PCR primers designed to amplify a 1043 bp product from the 5′ end of cosmid 21gD (product corresponds to nucleotides 70,572 to 71,614 of the complete biosynthetic cluster). The primers 21gDendFWD (5′-GCGACGAAGGACCCGAAGG; SEQ ID NO:164) and 21gDendREV (5′-CACGCTGGCCCGCCCCTTC; SEQ ID NO:165) were used to screen each of the cosmids using 10-100 ng of each cosmid as template in a standard 25 μl PCR reaction (KOD Hot Start polymerase; Novagen, San Diego, Calif., USA) along with 0.5 μM of each primer. The only cosmids that supported amplification of the expected 1043 bp DNA fragment were cosmids 21gB and 21gC. End sequencing of these cosmids revealed that cosmid 21gB overlapped cosmid 21gD by 17,411 nucleotides, while cosmid 21gC overlapped cosmid 21gD by 22,796 nucleotides. Since cosmid 21gB overlapped less with the known cluster sequence, and thereby represented a greater potential for yielding a longer sequence extension than cosmid 21gC, it was chosen for sequencing. Sequencing revealed that cosmid 21gB contained a 33,133 bp insert which represented a 18,442 bp sequence extension, bringing the total number of base pairs sequenced to 90,573 (FIG. 7). As before, the cosmid was sequenced, open reading frames were identified, and functions of the encoded proteins were deduced.

Biological Properties of the 21G792 Chromoprotein

### Example 4

### In Vitro Anti-Tumor Activity

The p53/p21 checkpoint monitors the integrity of the genome and blocks cell cycle progression in the event of DNA damage. Disruption of the checkpoint by deletion of the p21 gene results in failure to arrest in response to DNA damage ultimately leading to cell death through apoptosis. Since loss of this checkpoint is a hallmark of cancer cells, an isogenic pair of cell lines, wherein one pair of the cell line (p21+/+) has an intact p21 gene and one member (p21−/−) has a deletion in the p21 gene, can be used to screen for potential anti-tumor compounds by identifying molecules that preferentially induce apoptosis in p21-deficient cells.

The Actinomadura sp. 21G792 chromoprotein was added to an isogenic pair of cell lines (p21+/+ and p21−/−). As shown in Table 6, the chromoprotein was highly selective for p21−/− cells, as the IC₅₀ was 13-fold higher for p21+/+ cells. Also, as shown in Table 7, the chromoprotein showed excellent potency in a human tumor cell line panel, as the IC₅₀ ranged from 1 to 47 ng/ml. The apoprotein alone, however, was inactive.

TABLE 6
Sensitivity of p21−/− Cells to Actinomadura sp. 21G792 Chromoprotein

Isogenic cell lines

| | p21+/+ | p21−/− | Selectivity Ratio |
|---|---|---|---|
| IC₅₀ (μg/ml) | 90 ± 32 | 7 ± 2 | 13 |

Mean ± SD, n = 3

TABLE 7
Potency of Actinomadura sp. 21G792 Chromoprotein Against
Human Tumor Cell Lines

| Tumor Cell Line | Tissue | IC₅₀ (μg/ml) |
|---|---|---|
| DLD1 | Colon | 8 |
| HCT116 | Colon | 1 |
| HT29 | Colon | 8 |
| LoVo | Colon | 2 |
| SW620 | Colon | 2 |
| BT474 | Breast | 47 |
| MCF-7 | Breast | 2 |
| MDA-MB-361 | Breast | 5 |
| HN5 | Head & Neck | 4 |
| LOX | Melanoma | 1 |
| PC3 | Prostate | 22 |

### Example 5

### DNA Damage Induced by the Chromoprotein

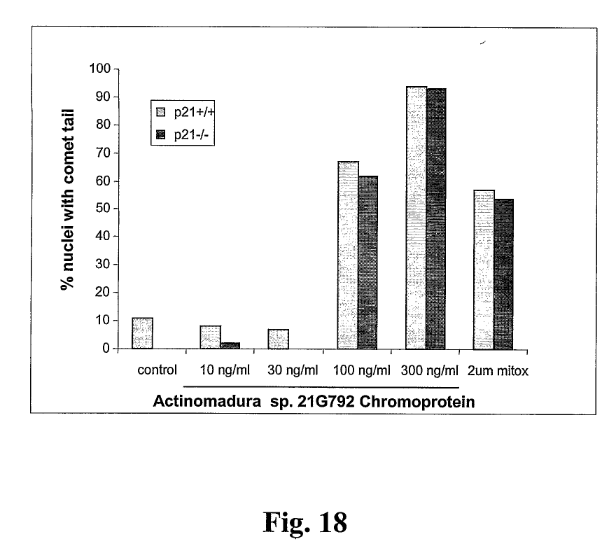

A COMET assay obtained from Trevigen, Inc. was used to detect DNA damage. HCT116 p21+/+ and −/− cells were subjected to various amounts of the 21G792 chromoprotein and mitoxantrone. As shown in FIG. 18, the chromoprotein induced dose-dependent DNA strand breaks occur in both p21-proficient and p21-deficient cells at >100 ng/ml concentrations.

### Example 6

### DNA Cleavage Induced by the Chromoprotein

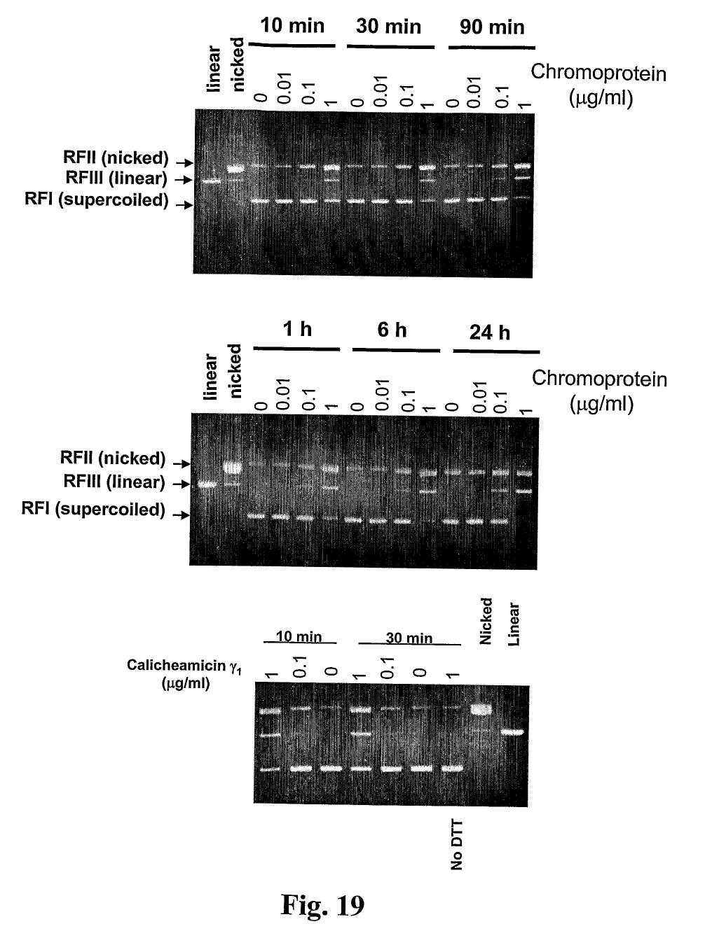

Supercoiled φX174 DNA was incubated with various concentrations of the 21G792 chromoprotein and analyzed by gel electrophoresis. It was observed that the chromoprotein induced single strand breaks and double strand breaks, the reaction continued to progress over 24 hours, and DNA cleavage did not require a reducing agent (dithiothreitol, DTT), unlike calicheamicin. The gel electrophoresis is shown in FIG. 19. Nicked refers to single strand breaks in the DNA and linear refers to double strand breaks.

### Example 7

### Digestion of Histone H1 by the Chromoprotein

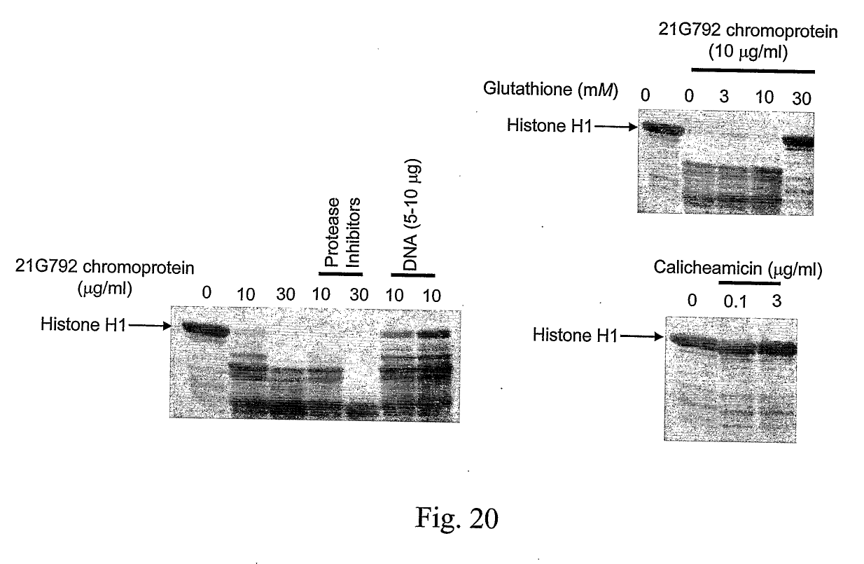

Chromoprotein enediynes have previously been shown to cleave histones (Zein et al., 1993, Proc. Natl. Acad. Sci. USA 90, 8009-12; Zein et al, 1995, Chem & Biol 2, 451-5; Zein et al., 1995, Biochem 34, 11591-7), and although this activity is controvorsial (Heyd et al., 2000, J. Bacteriol. 182, 1812-8), it was presumed to be due to a proteolytic activity of the apoprotein. Histone H1 was incubated with various concentrations of the chromoprotein in 50 mM Tris-Cl, pH 7.5 overnight at 37° C. (FIG. 20) Digestions of histone were assessed by SDS-polyacrylamide gel electrophoresis (SDS-PAGE), followed by staining of the gel with GelCode Blue (Pierce Biotechnology, Inc, Rockford, Ill.). Digestion of histone HI was inhibited by addition of DNA, indicating that the same mechanisms required for DNA cleavage (e.g., a free-radical based mechanism) are also involved in digesting proteins. Consistent with this, digestion of histones was inhibited by the addition of free radical scavengers, 30 mM glutathione or N-acetyl cysteine (not shown), but not by protease inhibitors. Calicheamicin, a non-protein-containing enediyne, did not cleave histone H1, indicating the requirement of an intact chromophore-protein complex for this activity.

### Example 8

### Specificity of Digestion by the Chromoprotein

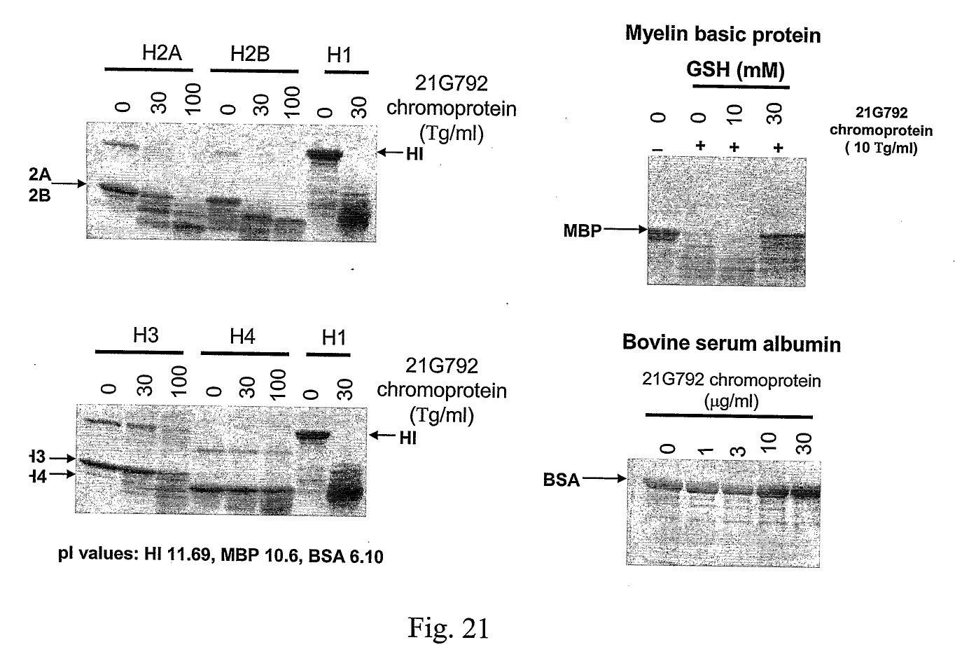

The order of preference of digestion of histones by the chromoprotein is H1>H2A>H2B>H3>H4 (FIG. 21). The chromoprotein also cleaves other basic proteins such as myelin basic protein, but not neutral/acidic proteins such as bovine serum albumin. This can explain the requirement of the apoprotein component of the chromophore for histone cleaving activity: the acidic apoprotein may deliver the chromophore to histones and other basic proteins by electrostatic interaction, allowing the chromophore to cleave the basic proteins by a free-radical based mechanism.

### Example 9

### Digestion of Histone H1 in HeLa Cells by the Chromoprotein

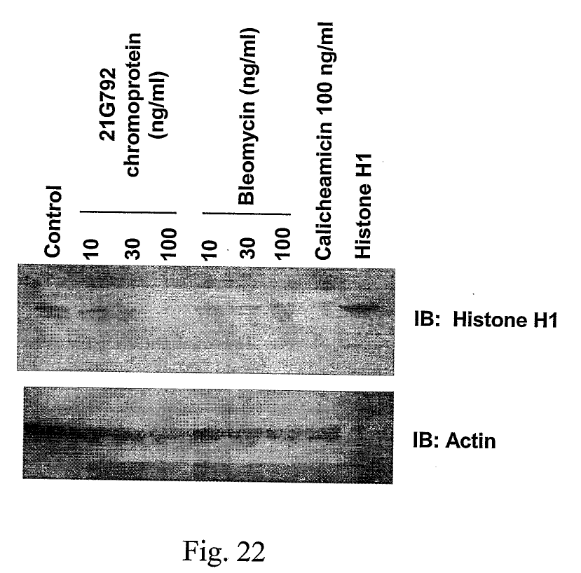

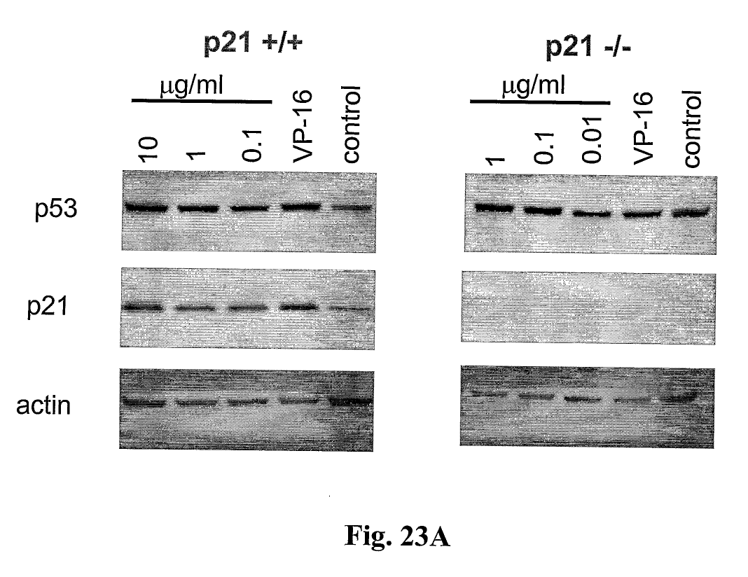

To study whether the digestion of histones by the chromoprotein occurs in intact cells, HeLa cells were incubated with compounds overnight at 37° C. Cell lysates were analysed by SDS-PAGE and protein immunoblotting using anti-histone H1 antibodies (Santa Cruz Biotechnologies). Incubation of cells with the chromoprotein resulted in reduced histone H1 in cells (FIG. 22). No effect was observed with bleomycin, another DNA damaging agent, or with calicheamicin. This demonstrates that the chromoprotein is capable of digesting histones within intact cells. This activity can contribute to antitumor effects by digesting histones in chromatin, making the DNA more accessible for cleavage. This appears to be a unique activity of the chromoprotein enediynes.

### Example 10

### Chromoprotein Induction of the G1/S Checkpoint

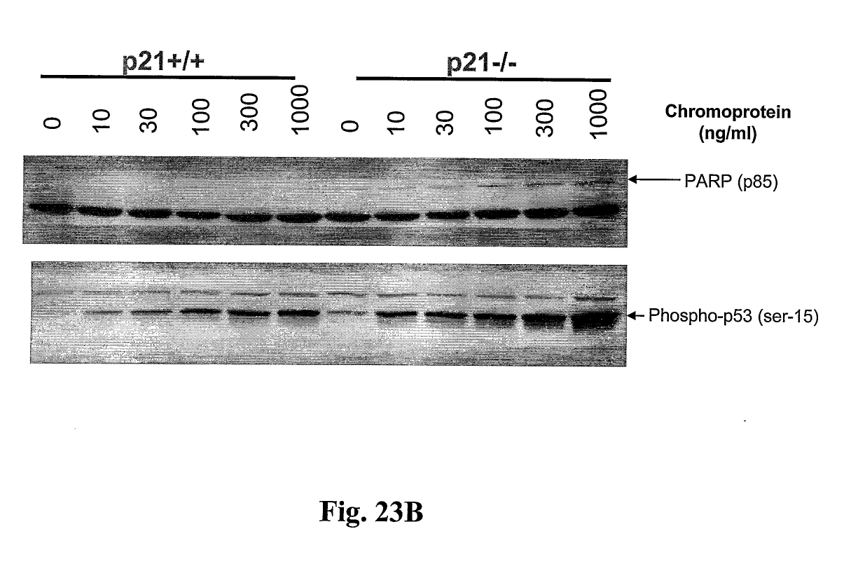

HCT116 (p21+/+ and p21−/−) cells were exposed to the chromoprotein at various concentrations. As shown in FIG. 23A, exposure to the chromoprotein resulted in the activation of the p53 checkpoint for all tested concentrations. Induction of the p21 protein was seen in the p21+/+ cells only. Activation of the DNA damage checkpoint by the Actinomadura sp. 21G792 chromoprotein was confirmed by demonstrating phosphorylation of the serine-15 amino acid residue in p53, which is known to be important for the transcriptional activation of the p53 protein (FIG. 23B). Furthermore, induction of apoptosis was preferentially observed in p21−/− cells compared with p21+/+ cells, when treated with the Actinomadura sp. 21G792 chromoprotein as shown by the cleavage of poly ADP ribose phosphorylase (PARP) (FIG. 23B). This is consistent with the lower IC50 value in the p21−/− cells.

### Example 11

### In Vivo Anti-Tumor Activity

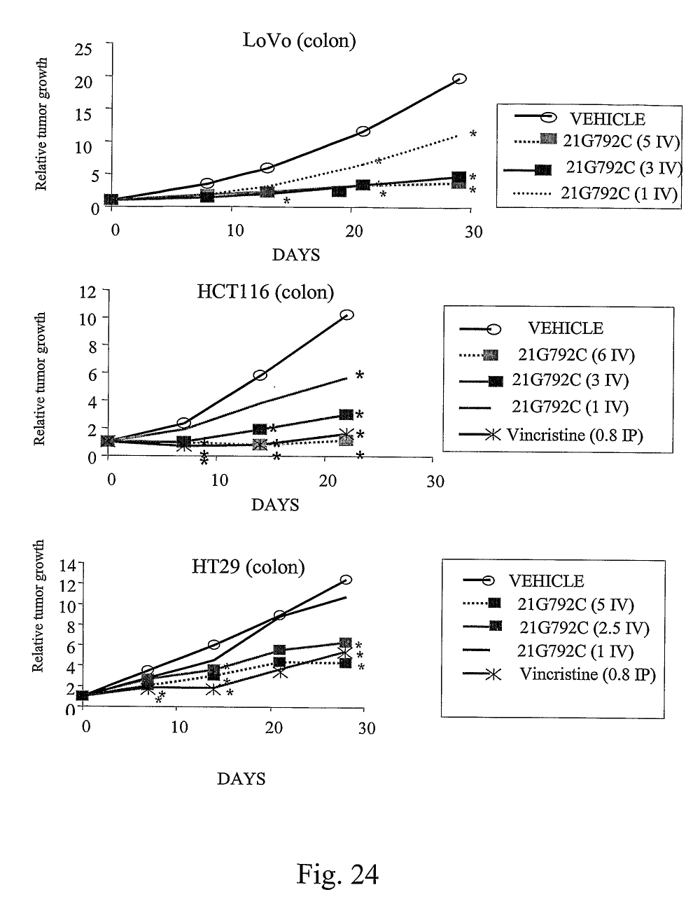

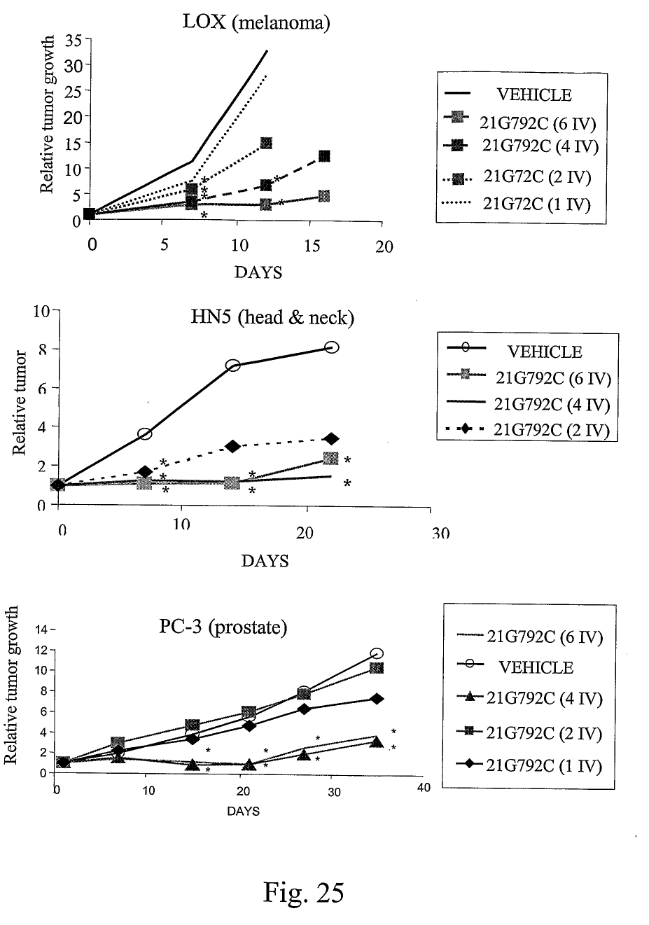

The human tumor cell lines or fragments LoVo (colon cancer); HCT116 (colon); HT29 (colon); LOX (melanoma); HN5 (head & neck); and PC-3 (prostate) were implanted under the skin of athymic (nude) mice and allowed to form a tumor mass. When the tumors reached a size of 90-200 mg, the saline control vehicle or various concentrations of the Actinomadura sp. 21G792 chromoprotein formulated in saline was administered intravenously to the mice. The mice received subsequent doses on days 5 and 9 and the relative tumor growth was observed. The results are shown in the graphs in FIG. 24 and FIG. 25. Inhibition of tumor growth of up to 80% for mice receiving the chromoprotein was observed.

### Example 12

### Toxicity of the Chromoprotein

Toxicology studies suggest that, except for bone marrow suppression, the Actinomadura sp. 21G792 chromoprotein is well-tolerated in nude mice. Specifically, saline control vehicle or the chromoprotein in various doses was administered intravenously to six nude mice on days 1, 5, and 9. Microscopic studies of the mice showed that all mice receiving the chromoprotein exhibited bone marrow necrosis, with the mice receiving the most chromoprotein exhibiting the most severe lesions. A clinical pathology experiment revealed that mice receiving the most chromoprotein exhibited the lowest number of white blood cells and lymphocytes. No adverse effects, however were observed in the intestine, nerves, spinal cord, liver, or at the site of injection. The microscopic finding and clinical pathology summaries are provided in Tables 8 and 9.

TABLE 8
Microscopic Finding Summary

| | | | Bone Marrow |
|---|---|---|---|
| Group | Treatment | Dose (mg/kg) | Necrosis^{a} |
| 1 | Vehicle | 0 | 0/6 |
| 2 | 21G792 | 3 | 6/6 (1.7) |
| 3 | 21G792 | 6 | 6/6 (3) |

^{a}number with lesion/total number examined(x): average lesion severity where 0 = WNL, 1 = slight, 2 = mild, 3 = moderate, 4 = marked, 5 = severe

TABLE 9
Clinical Pathology

| | | | | Lymphocytes |
|---|---|---|---|---|
| Group | Treatment | Dose (mg/kg) | WBC (cells/μl) | (cells/μl) |
| 1 | Vehicle | 0 | 5100 | 3900 |
| 2 | 21G792 | 3 | 1430 | 290 |
| 3 | 21G792 | 6 | 1280 | 40 |

### Example 13

### Transport of the Chromoprotein by P-GP (MDR-1)

Human PGP (MDR1) is an ATP-dependent efflux pump which is capable of transporting many drugs across cell membranes. High level expression of this protein has been linked to multiple drug resistance of tumors. As shown in Table 10 below, the Actinomadura sp. 21G792 chromoprotein is a poor MDR1 substrate, and cells expressing clinically relevant levels of MDR1 (KB-8-5 cells) remain sensitive to the complex. Notably, calicheamicin, which does not have a protein component, is a good substrate for MDR1. The protein component of the chromoprotein probably protects the chromophore from drug efflux mediated by MDR1, and may be responsible for the beneficial antitumor effects in colon cell lines which often express MDR1.

TABLE 10
IC₅₀ of Actinomadura sp. 21G792 Chromophore and
Calicheamicin Against P-GP Expressing Cells

IC₅₀ (ng/ml)^{a}

| Cell Line | P-GP Levels | 21G792 | Calicheamicin |
|---|---|---|---|
| KB | − | 10 | 3 |
| KB-8-5 | + | 6 | 21 |
| KB-V-1 | +++ | 142 | >1000 |

^{a}mean of two independent experiments

### Example 14

### Uptake of FITC-Tagged Chromoprotein in HCT116 Cells

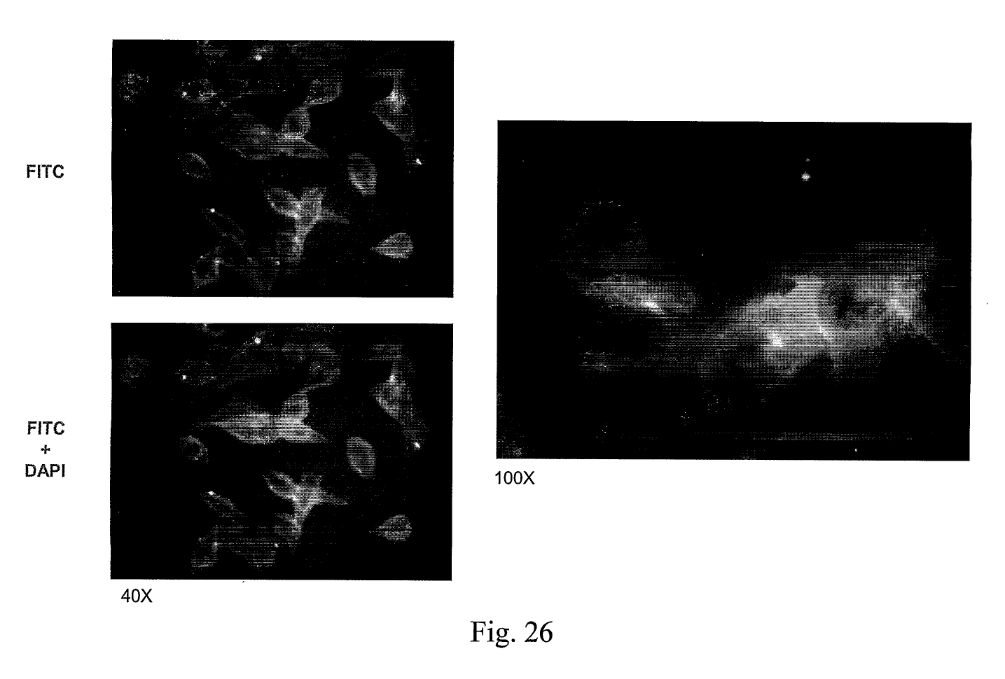

To determine the mechanism by which the chromoprotein enters cells and exerts its biological activity, the chromoprotein was labeled with a fluorescent tag (FITC) using EZ-Label fluorescent labeling kit (Pierce Biotechnology), according to the manufacturer's recommendation. No loss of biological activity was observed upon labeling. Uptake of labeled material by HCT116 colon carcinoma cells was studied by fluorescent microscopy. Optimum incubation time with cells was 3-6 hours. Most of the label appeared in the cytoplasm, although weak staining was also observed in the nucleus (FIG. 26). Even though nuclear accumulation is low, the amount is most likely sufficient for biological activity given the potency of the complex.

### Example 15

### Uptake of FITC-Tagged Apoprotein and Chromoprotein in HCT116 Cells

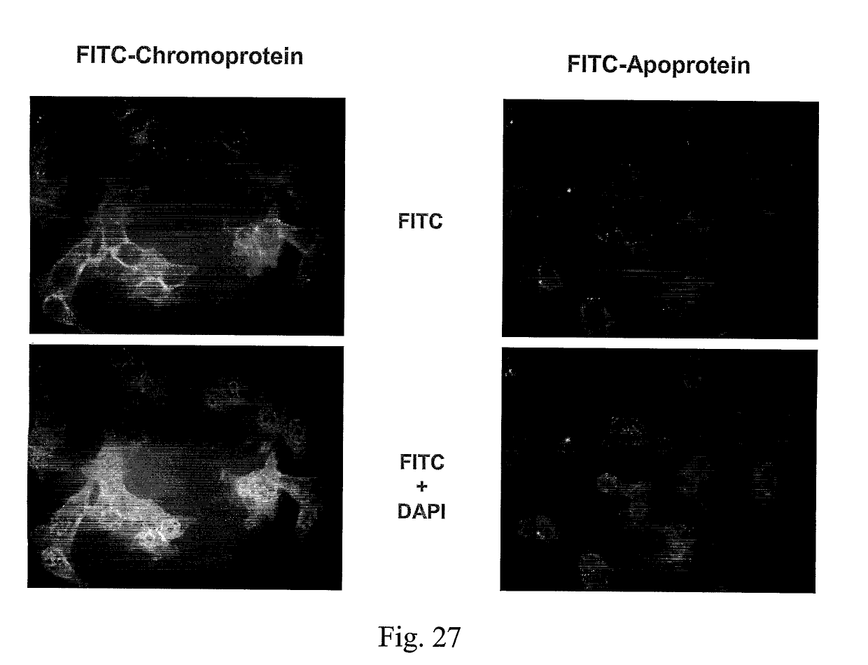

To determine whether an intact complex of chromophore and apoprotein is required for cellular and nuclear entry, the chromoprotein and apoprotein were labeled with FITC. Uptake of labeled material was studied by fluorescent microscopy. Uptake was similar for both apoprotein and chromoprotein. (FIG. 27), suggesting that cellular entry is not dependent on an intact chromophore-protein complex.

### Example 16

### Uptake of FITC-Tagged Chromoprotein: Competition with Unlabeled Complex

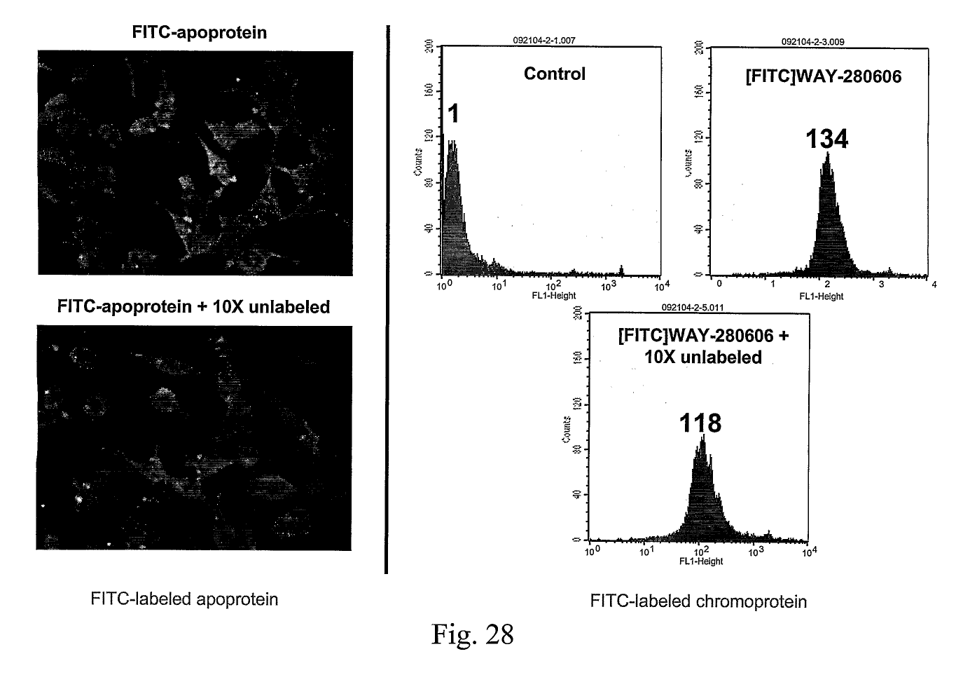

To determine whether the entry of chromoprotein into cells is mediated by a saturable (e.g. cell surface receptor-dependent) process, HCT116 cells were incubated with FITC-labeled chromoprotein (FIG. 28, right panel) or apoprotein (FIG. 28, left panel) in the absence or presence of 10-fold excess of unlabeled reagent (unlabelled chromoprotein or apoprotein, respectively). Cells were analysed by fluorescent microscopy (left) or flow cytometry (right). No competition of label was observed, suggesting that uptake of labeled material was not a receptor-mediated process. Furthermore, a single homogeneous peak observed in flow cytometry histograms indicated uniform uptake of labeled reagent by all cells. Numbers in the histograms are mean channel numbers (FITC fluorescence).

### Example 17

### Effect of Energy Depletion and Microtubule Disruption on Uptake of FITC-Tagged Apoprotein by HCT116 Cells

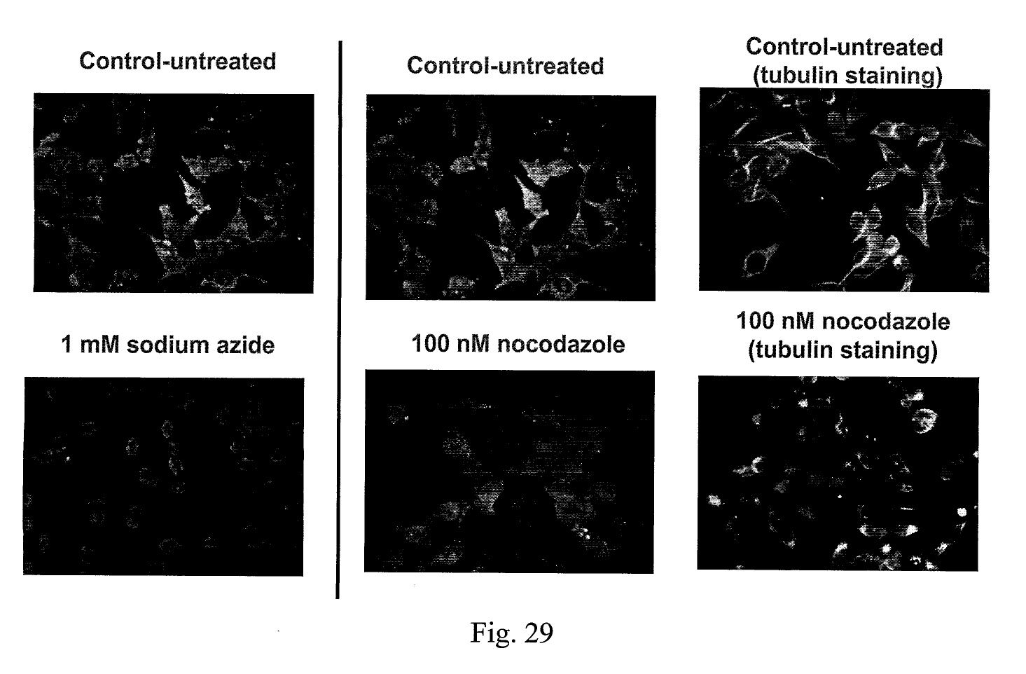

The above experiments suggest that entry of chromoprotein into cells is not a receptor-mediated process. Other means by which a protein complex can enter cells is pinocytosis, where caveolae in the surface of the cell pinch off to form pinosomes that are free within the cytoplasm of the cell. Since pinocytosis is an energy-dependent process that requires a functional tubulin cytoskeletal network, we examined the effect of sodium azide, an energy uncoupling agent and nocodazole, an agent which disrupts the tubulin cytoskeleton on cellular uptake. HCT116 cells were treated with FITC-labeled apoprotein in the absence or presence of sodium azide or nocodazole. Both treatments inhibited uptake of label (FIG. 29). The concentration of nocodazole (100 mM) was shown to be sufficient to disrupt microtubules (right panels). These data suggest that uptake of apoprotein is an energy-dependent process utilizing the microtubule network. Since our data appears to rule out a receptor-mediated process, pinocytosis is most likely involved.

1. An isolated nucleic acid comprising a nucleotide sequence that is at least about 70% identical to the nucleotide sequence of an orf of the chromoprotein biosynthetic gene cluster of Actinomadura sp. 21G792 (NRRL 30778) having SEQ ID NO:1, SEQ ID NO:3, SEQ ID NO:5, SEQ ID NO:7, SEQ ID NO:9, SEQ ID NO:11, SEQ ID NO:13, SEQ ID NO:15, SEQ ID NO:17, SEQ ID NO:19, SEQ ID NO:21, SEQ ID NO:23, SEQ ID NO:25, SEQ ID NO:27, SEQ ID NO:29, SEQ ID NO:31, SEQ ID NO:33, SEQ ID NO:35, SEQ ID NO:37, SEQ ID NO:39, SEQ ID NO:41, SEQ ID NO:43, SEQ ID NO:45, SEQ ID NO:47, SEQ ID NO:49, SEQ ID NO:51, SEQ ID NO:53, SEQ ID NO:55, SEQ ID NO:57, SEQ ID NO:59, SEQ ID NO:61, SEQ ID NO:63, SEQ ID NO:65, SEQ ID NO:67, SEQ ID NO:69, SEQ ID NO:71, SEQ ID NO:73, SEQ ID NO:75, SEQ ID NO:77, SEQ ID NO:79, SEQ ID NO:81, SEQ ID NO:83, SEQ ID NO:85, SEQ ID NO:87, SEQ ID NO:89, SEQ ID NO:91, SEQ ID NO:93, SEQ ID NO:95, SEQ ID NO:97, SEQ ID NO:99, SEQ ID NO:101, SEQ ID NO:103, SEQ ID NO:105, SEQ ID NO:107, SEQ ID NO:109, SEQ ID NO:111, SEQ ID NO:113, SEQ ID NO:115, SEQ ID NO:117, SEQ ID NO:119, SEQ ID NO:121, SEQ ID NO:123, SEQ ID NO:125, SEQ ID NO:127, SEQ ID NO:129, SEQ ID NO:131, SEQ ID NO:133, SEQ ID NO:135, SEQ ID NO:137, SEQ ID NO:139, SEQ ID NO:141, SEQ ID NO:143, SEQ ID NO:145, SEQ ID NO:147, or SEQ ID NO:149, or the complement thereof.

2. The isolated nucleic acid of claim 1, wherein the isolated nucleotide sequence is identical to the nucleotide sequence of an orf of the chromoprotein biosynthetic gene cluster of Actinomadura sp. 21G792 (NRRL 30778).

3. The isolated nucleic acid of claim 1, which comprises the chromoprotein biosynthetic gene cluster having SEQ ID NO:151.

4. An isolated nucleic acid that comprises a sequence that encodes the amino acid sequence of an orf of the chromoprotein biosynthetic gene cluster of Actinomadura sp. 21G792 (NRRL 30778) having SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ ID NO:20, SEQ ID NO:22, SEQ ID NO:24, SEQ ID NO:26, SEQ ID NO:28, SEQ ID NO:30, SEQ ID NO:32, SEQ ID NO:34, SEQ ID NO:36, SEQ ID NO:38, SEQ ID NO:40, SEQ ID NO:42, SEQ ID NO:44, SEQ ID NO:46, SEQ ID NO:48, SEQ ID NO:50, SEQ ID NO:52, SEQ ID NO:54, SEQ ID NO:56, SEQ ID NO:58, SEQ ID NO:60, SEQ ID NO:62, SEQ ID NO:64, SEQ ID NO:66, SEQ ID NO:68, SEQ ID NO:70, SEQ ID NO:72, SEQ ID NO:74, SEQ ID NO:76, SEQ ID NO:78, SEQ ID NO:80, SEQ ID NO:82, SEQ ID NO:84, SEQ ID NO:86, SEQ ID NO:88, SEQ ID NO:90, SEQ ID NO:92, SEQ ID NO:94, SEQ ID NO:96, SEQ ID NO:98, SEQ ID NO:100, SEQ ID NO:102, SEQ ID NO:104, SEQ ID NO:106, SEQ ID NO:108, SEQ ID NO:110, SEQ ID NO:112, SEQ ID NO:114, SEQ ID NO:116, SEQ ID NO:118, SEQ ID NO:120, SEQ ID NO:122, SEQ ID NO:124, SEQ ID NO:126, SEQ ID NO:128, SEQ ID NO:130, SEQ ID NO:132, SEQ ID NO:134, SEQ ID NO:136, SEQ ID NO:138, SEQ ID NO:140, SEQ ID NO:142, SEQ ID NO:144, SEQ ID NO:146, SEQ ID NO:148, or SEQ ID NO:150.

5. The nucleic acid of claim 1 that encodes an apoprotein.

6. The nucleic acid of any of claim 1 that encodes a preapoprotein.

7. A vector comprising the nucleic acid of claim 1.

8. The vector of claim 7, wherein the nucleic acid is operably linked to a regulatory nucleic acid sequence that controls gene expression.

9. The vector of claim 7, wherein gene expression is constitutive or inducible.

10. The vector of claim 7, wherein the vector is a cosmid.

11. A host cell comprising the nucleic acid of claim 1.

12. A host cell comprising the vector of claim 7.

13. The host cell of claim 12, wherein the host cell is a prokaryotic cell.

14. The host cell of claim 13, wherein the prokaryotic cell is of a genus selected from the group consisting of Actinomyces, Actinomadura, Streptomyces, or Micromonospora.

15. The host cell of claim 13, wherein the prokaryotic cell is Escherichia coli.

16. The host cell of claim 12, wherein the host cell is a eukaryotic cell.

17. A method of expressing a protein comprising transfecting a host cell with the vector of claim 7 and incubating the cell under conditions suitable for expression of the protein.

18. An isolated polypeptide comprising the amino acid sequence having at least about 70% homology to SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ ID NO:20, SEQ ID NO:22, SEQ ID NO:24, SEQ ID NO:26, SEQ ID NO:28, SEQ ID NO:30, SEQ ID NO:32, SEQ ID NO:34, SEQ ID NO:36, SEQ ID NO:38, SEQ ID NO:40, SEQ ID NO:42, SEQ ID NO:44, SEQ ID NO:46, SEQ ID NO:48, SEQ ID NO:50, SEQ ID NO:52, SEQ ID NO:54, SEQ ID NO:56, SEQ ID NO:58, SEQ ID NO:60, SEQ ID NO:62, SEQ ID NO:64, SEQ ID NO:66, SEQ ID NO:68, SEQ ID NO:70, SEQ ID NO:72, SEQ ID NO:74, SEQ ID NO:76, SEQ ID NO:78, SEQ ID NO:80, SEQ ID NO:82, SEQ ID NO:84, SEQ ID NO:86, SEQ ID NO:88, SEQ ID NO:90, SEQ ID NO:92, SEQ ID NO:94, SEQ ID NO:96, SEQ ID NO:98, SEQ ID NO:100, SEQ ID NO:102, SEQ ID NO:104, SEQ ID NO:106, SEQ ID NO:108, SEQ ID NO:110, SEQ ID NO:112, SEQ ID NO:114, SEQ ID NO:116, SEQ ID NO:118, SEQ ID NO:120, SEQ ID NO:122, SEQ ID NO:124, SEQ ID NO:126, SEQ ID NO:128, SEQ ID NO:130, SEQ ID NO:132, SEQ ID NO:134, SEQ ID NO:136, SEQ ID NO:138, SEQ ID NO:140, SEQ ID NO:142, SEQ ID NO:144, SEQ ID NO:146, SEQ ID NO:148, or SEQ ID NO:150.

19. The isolated polypeptide of claim 18, wherein the amino acid sequence is identical to SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ ID NO:20, SEQ ID NO:22, SEQ ID NO:24, SEQ ID NO:26, SEQ ID NO:28, SEQ ID NO:30, SEQ ID NO:32, SEQ ID NO:34, SEQ ID NO:36, SEQ ID NO:38, SEQ ID NO:40, SEQ ID NO:42, SEQ ID NO:44, SEQ ID NO:46, SEQ ID NO:48, SEQ ID NO:50, SEQ ID NO:52, SEQ ID NO:54, SEQ ID NO:56, SEQ ID NO:58, SEQ ID NO:60, SEQ ID NO:62, SEQ ID NO:64, SEQ ID NO:66, SEQ ID NO:68, SEQ ID NO:70, SEQ ID NO:72, SEQ ID NO:74, SEQ ID NO:76, SEQ ID NO:78, SEQ ID NO:80, SEQ ID NO:82, SEQ ID NO:84, SEQ ID NO:86, SEQ ID NO:88, SEQ ID NO:90, SEQ ID NO:92, SEQ ID NO:94, SEQ ID NO:96, SEQ ID NO:98, SEQ ID NO:100, SEQ ID NO:102, SEQ ID NO:104, SEQ ID NO:106, SEQ ID NO:108, SEQ ID NO:110, SEQ ID NO:112, SEQ ID NO:114, SEQ ID NO:116, SEQ ID NO:118, SEQ ID NO:120, SEQ ID NO:122, SEQ ID NO:124, SEQ ID NO:126, SEQ ID NO:128, SEQ ID NO:130, SEQ ID NO:132, SEQ ID NO:134, SEQ ID NO:136, SEQ ID NO:138, SEQ ID NO:140, SEQ ID NO:142, SEQ ID NO:144, SEQ ID NO:146, SEQ ID NO:148, or SEQ ID NO:150.

20. The isolated polypeptide of claim 18, wherein the polypeptide is an apoprotein and is capable of forming a non-covalent complex with a chromophore.

21. The isolated polypeptide of claim 20, wherein the complex is capable of cleavage of single- or double-stranded DNA.

22. The isolated polypeptide of claim 20, wherein the chromophore is from Actinomadura sp. 21G792.

23. An isolated chromoprotein comprising a non-covalent complex of the polypeptide of claim 20 and the chromophore of Actinomadura sp. 21G792 (NRRL 30778).

24. An oligonucleotide that specifically hybridizes to a DNA molecule having the nucleotide sequence of SEQ ID NO:151, or the complement thereof.

25. The oligonucleotide of claim 24, which is selected from the group consisting of SEQ ID NO:158, SEQ ID NO:159, SEQ ID NO:160, SEQ ID NO:161, SEQ ID NO:162, SEQ ID NO:163, and the complementary sequences thereof.

26. The oligonucleotide of claim 24, which is degenerate and is selected from the group consisting of SEQ ID NO:155, SEQ ID NO:156, SEQ ID NO:157, and the complementary sequences thereof.

27. A method of identifying a nucleic acid that encodes an apoprotein of a nine-membered enediyne containing chromoprotein which comprises contacting the nucleic acid with the oligonucleotide of any one of claims 24 and detecting specific hybridization of the oligonucleotide to the nucleic acid.

28. A method of identifying a nucleic acid that encodes an apoprotein of a nine-membered enediyne containing chromoprotein which comprises contacting the nucleic acid with oligonucleotides having SEQ ID NO:156 and SEQ ID NO:157 and detecting specific hybridization by amplification.

29. The method of claim 27, wherein the nucleic acid is from an organism of the order Actinomycetales.

30. The method of claim 29, wherein the organism is of a genus selected from the group consisting of Actinomyces, Actinomadura, Streptomyces, or Micromonospora.

31. The method of claim 29, wherein the organism is Actinomadura sp. 21G792 (NRRL 30778).

32. A biologically pure culture of Actinomadura sp. 21G792 (NRRL 30778) capable of producing an apoprotein having SEQ ID NO:150.

33. A method of making a chromoprotein comprising incubating Actinomadura sp. 21G792 (NRRL 30778) in a culture medium under conditions suitable for expression of the chromoprotein and recovering the chromoprotein from the culture medium.

34. A method of making a modified chromoprotein comprising:
a) subjecting a plurality of first polynucleotides comprising a selected orf of Actinomadura sp. 21G792 to simultaneous mutagenesis so as to produce a plurality of progeny polynucleotides;
b) expressing polypeptides from the progeny polynucleotides in host cells that produce an enediyne chromophore; and
c) selecting or screening the host cells for polypeptide/chromophore complexes having a desired characteristic, thereby identifying a modified chromoprotein.

35. The method of claim 34, wherein the first off is selected from the group consisting of orf15, orf19, orf20, orf32, orf33, and orf40.

36. The method of claim 34, wherein the first off is orf23.

37. The method of claim 34, wherein (a) further comprises subjecting a plurality of second polynucleotides comprising a second selected off of Actinomadura sp. 21G792 to simultaneous mutagenesis so as to produce a plurality of progeny polynucleotides.

38. The method of claim 35, wherein the second off is selected from the group consisting of orf15, orf19, orf20, orf23, orf32, orf33, and orf40.

39. The method of claim 38, wherein the first off or the second off is orf23.

40. The method of claim 34, wherein the desired characteristic is inactivation of at least one chromophore biosynthetic enzyme.

41. The method of claim 40, wherein Orf32 is inactivated.

42. The method of claim 41, which further comprises culturing the host cell in a fermentation broth comprising a benzoic acid analog.

43. The method of claim 34, wherein the host cell is Actinomadura sp. 21G792 (NRRL 30778).

44. The method of claim 34, wherein the host cell is a heterologous host cell.

45. A method of inhibiting progression of a neoplastic disease in a mammal comprising administering to the mammal an effective amount of the chromoprotein of Actinomadura sp. 21G792 (NRRL 30778).

46. The method of claim 45, wherein the neoplastic disease is selected from the group consisting of colon cancer, breast cancer, melanoma, head and neck cancer, and prostate cancer.

47. A pharmaceutical composition comprising an effective amount of the chromoprotein of claim 23 and a pharmaceutically acceptable carrier.

48. A compound having the formula: 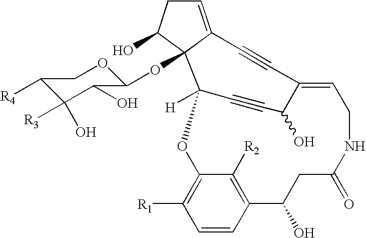
wherein R¹ is OH or OCH₃; R² is Cl or H; R³ is CH₃ or H; R⁴ is selected from NH₂, R⁵ and R⁶;
wherein R⁵ is 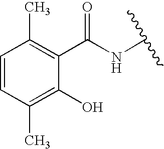
and R⁶ is

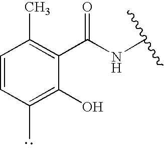

49. The compound of claim 48, wherein R¹ is OCH₃, R² is Cl, R³ is CH₃, and R₄ is R⁵.

50. The compound of claim 48, wherein R¹ is OCH₃, R² is H, R³ is CH₃, and R⁴ is R⁵.

51. The compound of claim 48, wherein R¹ is OCH₃, R² is Cl, R³ is H, and R⁴ is R⁵.

52. The compound of claim 48, wherein R¹ is OCH₃, R² is Cl, R³ is CH₃, and R⁴ is NH₂.

53. The compound of claim 48, wherein R¹ is OCH₃, R² is Cl, R³ is CH₃, and R⁴ is R⁶.

54. The compound of claim 48, wherein R¹ is OCH₃, R² is H, R³ is H, and R⁴ is R⁵.

55. The compound of claim 48, wherein R¹ is OCH₃, R² is H, R³ is H, and R⁴ is NH₂.

56. The compound of claim 48, wherein R¹ is OCH₃, R² is H, R³ is H, and R⁴ is R⁶.

57. The compound of claim 48, wherein R¹ is OCH₃, R² is Cl, R³ is H, and R⁴ is NH₂.

58. The compound of claim 48, wherein R¹ is OCH₃, R² is Cl, R³ is H, and R⁴ is R⁶.

59. The compound of claim 48, wherein R¹ is OCH₃, R² is H, R³ is CH₃, and R⁴ is NH₂.

60. The compound of claim 48, wherein R¹ is OCH₃, R² is H, R³ is CH₃, and R⁴ is R⁶.

61. The compound of claim 48, wherein R¹ is OH, R² is Cl, R³ is CH₃, and R⁴ is R⁵.

62. The compound of claim 48, wherein R¹ is OH, R² is H; R³ is CH₃, and R⁴ is R⁵.

63. The compound of claim 48, wherein R¹ is OH, R² is Cl, R³ is H, and R⁴ is R⁵.

64. The compound of claim 48, wherein R¹ is OH, R² is Cl, R³ is CH₃, and R⁴ is NH₂.

65. The compound of claim 48, wherein R¹ is OH, R² is Cl, R³ is CH₃, and R⁴ is R⁶.

66. The compound of claim 48, wherein R¹ is OH, R² is H, R³ is H, and R⁴ is R⁵.

67. The compound of claim 48, wherein R¹ is OH, R² is H, R³ is H, and R⁴ is NH₂.

68. The compound of claim 48, wherein R¹ is OH, R² is H, R³ is H, and R⁴ is R⁶.

69. The compound of claim 48, wherein R¹ is OH, R² is Cl, R³ is H, and R⁴ is NH₂.

70. The compound of claim 48, wherein R¹ is OH, R² is Cl, R³ is H, and R⁴ is R⁶.

71. The compound of claim 48, wherein R¹ is OH, R² is H, R³ is CH₃, and R⁴ is NH₂.

72. The compound of claim 48, wherein R¹ is OH, R² is H, R³ is CH₃, and R⁴ is R⁶.

73. A compound having the formula: 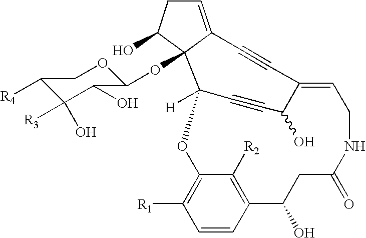
wherein R¹ is OH or OCH₃; R² is Cl or H; R³ is CH₃ or H; R⁴ is selected from R⁷ and R⁸;
wherein R⁷ is 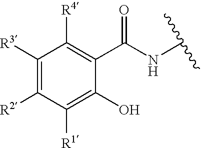
and R⁸ is 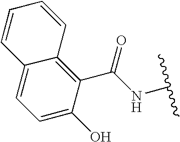
and wherein R^{1′} is H, CH₃, OH, OCH₃, Cl, C₃H₇, or NO₂; R^{2′} is H, CH₃, NH₂, OH, F, OCH₃, F, Cl, NO₂, OC₂H₅, or NC₂H₆; R^{3′} is H, CH₃, Cl, CH₃, NH₂, OH, F, COH, OCH₃, Cl, OC₂H₅, or NO₂; and R^{4′} is H, OH, or OCH₃.

74. The compound of claim 73, wherein R^{1′} is CH₃, R^{2′} is H, R^{3′} is CH₃, and R^{4′} is H.

75. The compound of claim 73, wherein R^{1′} is CH₃, R^{2′} is OH, R^{3′} is H, and R^{4′} is H.

76. The compound of claim 73, wherein R^{1′} is H, R^{2′} is CH₃, R^{3′} is H, and R^{4′} is OH.

77. The compound of claim 73, wherein R^{1′} is H, R^{2′} is OH, R^{3′} is OH, and R^{4′} is H.

78. The compound of claim 73, wherein R^{1′} is H, R^{2′} is OH, R^{3′} is H, and R^{4′} is OH.

79. The compound of claim 73, wherein R^{1′} is OH, R^{2′} is OH, R^{3′} is H, and R^{4′} is H.
